SEQUENCE LISTING (44 sequences)

SEQ ID NO: 1 - protein, 12 aa, synthetic construct
  source 1..12: note = YAP1 Inhibitor
KLRPVAMVRPVR

SEQ ID NO: 2 - protein, 24 aa, synthetic construct
  REGION 1..24: note = YAP1 Inhibitor
GRKKRRQRRRPQKLRPVAMVRPVR

SEQ ID NO: 3 - DNA, 20 bp, synthetic construct
  misc_feature 1..20: note = Forward primer for IL-6 gene promoter region
  source 1..20: mol_type = other DNA
ctgcaagttcccacagttca

SEQ ID NO: 4 - DNA, 20 bp, synthetic construct
  misc_feature 1..20: note = Reverse primer for IL-6 gene promoter region
  source 1..20: mol_type = other DNA
cccaccttcttcaaaatcca

SEQ ID NO: 5 - DNA, 21 bp, synthetic construct
  misc_feature 1..21: note = B-actin forward primer
  source 1..21: mol_type = other DNA
ttctacaatgagctgcgtgtg

SEQ ID NO: 6 - DNA, 20 bp, synthetic construct
  misc_feature 1..20: note = B-actin reverse primer
  source 1..20: mol_type = other DNA
ggggtgttgaaggtctcaaa

SEQ ID NO: 7 - DNA, 20 bp, synthetic construct
  misc_feature 1..20: note = ABCG2 forward primer
  source 1..20: mol_type = other DNA
agctgcaaggaaagatccaa

SEQ ID NO: 8 - DNA, 20 bp, synthetic construct
  misc_feature 1..20: note = ABCG2 reverse primer
  source 1..20: mol_type = other DNA
tccagacacaccacggataa

SEQ ID NO: 9 - DNA, 20 bp, synthetic construct
  misc_feature 1..20: note = ALDH1A1 forward primer
  source 1..20: mol_type = other DNA
tgttagctgatgccgacttg

SEQ ID NO: 10 - DNA, 20 bp, synthetic construct
  misc_feature 1..20: note = ALDH1A1 reverse primer
  source 1..20: mol_type = other DNA
ttcttagcccgctcaacact

SEQ ID NO: 11 - DNA, 16 bp, synthetic construct
  misc_feature 1..16: note = CD24 forward primer
  source 1..16: mol_type = other DNA
ctggcactgctcctac

SEQ ID NO: 12 - DNA, 16 bp, synthetic construct
  misc_feature 1..16: note = CD24 reverse primer
  source 1..16: mol_type = other DNA
gagtgagaccacgaag

SEQ ID NO: 13 - DNA, 20 bp, synthetic construct
  misc_feature 1..20: note = CTGF forward primer
  source 1..20: mol_type = other DNA
cctggtccagaccacagagt

SEQ ID NO: 14 - DNA, 20 bp, synthetic construct
  misc_feature 1..20: note = CTGF reverse primer
  source 1..20: mol_type = other DNA
tggagattttgggagtacgg

SEQ ID NO: 15 - DNA, 20 bp, synthetic construct
  misc_feature 1..20: note = CYR61 forward primer
  source 1..20: mol_type = other DNA
ctccctgtttttggaatgga

SEQ ID NO: 16 - DNA, 20 bp, synthetic construct
  misc_feature 1..20: note = CYR61 reverse primer
  source 1..20: mol_type = other DNA
tggtcttgctgcatttcttg

SEQ ID NO: 17 - DNA, 20 bp, synthetic construct
  misc_feature 1..20: note = EGF forward primer
  source 1..20: mol_type = other DNA
cagggaagatgaccaccact

SEQ ID NO: 18 - DNA, 20 bp, synthetic construct
  misc_feature 1..20: note = EGF reverse primer
  source 1..20: mol_type = other DNA
cagttcccaccacttcaggt

SEQ ID NO: 19 - DNA, 22 bp, synthetic construct
  misc_feature 1..22: note = IFN-gamma forward primer
  source 1..22: mol_type = other DNA
atgtattgctttgcgttggaca

SEQ ID NO: 20 - DNA, 26 bp, synthetic construct
  misc_feature 1..26: note = IFN-gamma reverse primer
  source 1..26: mol_type = other DNA
tcaatagcaacaaaaagaaacgagat

SEQ ID NO: 21 - DNA, 20 bp, synthetic construct
  misc_feature 1..20: note = IL-2 forward primer
  source 1..20: mol_type = other DNA
acctcaactcctgccacaat

SEQ ID NO: 22 - DNA, 20 bp, synthetic construct
  misc_feature 1..20: note = IL-2 reverse primer
  source 1..20: mol_type = other DNA
gccttcttgggcatgtaaaa

SEQ ID NO: 23 - DNA, 20 bp, synthetic construct
  misc_feature 1..20: note = IL-4 forward primer
  source 1..20: mol_type = other DNA
ggcagttctacagccaccat

SEQ ID NO: 24 - DNA, 20 bp, synthetic construct
  misc_feature 1..20: note = IL-4 reverse primer
  source 1..20: mol_type = other DNA
ctctggttggcttccttcac

SEQ ID NO: 25 - DNA, 20 bp, synthetic construct
  misc_feature 1..20: note = IL-6 forward primer
  source 1..20: mol_type = other DNA
atgtagccgccccacacaga

SEQ ID NO: 26 - DNA, 20 bp, synthetic construct
  misc_feature 1..20: note = IL-6 reverse primer
  source 1..20: mol_type = other DNA
catccatctttttcagccat

SEQ ID NO: 27 - DNA, 20 bp, synthetic construct
  misc_feature 1..20: note = IL-10 forward primer
  source 1..20: mol_type = other DNA
tgcaaaaccaaaccacaaga

SEQ ID NO: 28 - DNA, 20 bp, synthetic construct
  misc_feature 1..20: note = IL-10 reverse primer
  source 1..20: mol_type = other DNA
tctcggagatctcgaagcat

SEQ ID NO: 29 - DNA, 27 bp, synthetic construct
  misc_feature 1..27: note = NANOG forward primer
  source 1..27: mol_type = other DNA
cagctgtgtgtactcaatgatagattt

SEQ ID NO: 30 - DNA, 27 bp, synthetic construct
  misc_feature 1..27: note = NANOG reverse primer
  source 1..27: mol_type = other DNA
acaccattgctattcttcggccagttg

SEQ ID NO: 31 - DNA, 20 bp, synthetic construct
  misc_feature 1..20: note = NRP1 forward primer
  source 1..20: mol_type = other DNA
gggaacttggtggatgaatg

SEQ ID NO: 32 - DNA, 20 bp, synthetic construct
  misc_feature 1..20: note = NRP1 reverse primer
  source 1..20: mol_type = other DNA
tctatgaccgtgggcttttc

SEQ ID NO: 33 - DNA, 19 bp, synthetic construct
  misc_feature 1..19: note = OCT4 forward primer
  source 1..19: mol_type = other DNA
gtccgagtgtggttctgta

SEQ ID NO: 34 - DNA, 20 bp, synthetic construct
  misc_feature 1..20: note = OCT4 reverse primer
  source 1..20: mol_type = other DNA
ctcagtttgaatgcatggga

SEQ ID NO: 35 - DNA, 20 bp, synthetic construct
  misc_feature 1..20: note = PROS1 forward primer
  source 1..20: mol_type = other DNA
tcctggttctggaattgctc

SEQ ID NO: 36 - DNA, 20 bp, synthetic construct
  misc_feature 1..20: note = PROS1 reverse primer
  source 1..20: mol_type = other DNA
accaaggcaagcataacacc

SEQ ID NO: 37 - DNA, 23 bp, synthetic construct
  misc_feature 1..23: note = SOX2 forward primer
  source 1..23: mol_type = other DNA
cccacctacagcatgtcctactc

SEQ ID NO: 38 - DNA, 22 bp, synthetic construct
  misc_feature 1..22: note = SOX2 reverse primer
  source 1..22: mol_type = other DNA
tggagtgggaggaagaggtaac

SEQ ID NO: 39 - DNA, 21 bp, synthetic construct
  misc_feature 1..21: note = STAT3 forward primer
  source 1..21: mol_type = other DNA
atcacgccttctacagactgc

SEQ ID NO: 40 - DNA, 23 bp, synthetic construct
  misc_feature 1..23: note = STAT3 reverse primer
  source 1..23: mol_type = other DNA
catcctggagattctctaccact

SEQ ID NO: 41 - DNA, 19 bp, synthetic construct
  misc_feature 1..19: note = TGF-Beta forward primer
  source 1..19: mol_type = other DNA
cccagcatctgcaaagctc

SEQ ID NO: 42 - DNA, 20 bp, synthetic construct
  misc_feature 1..20: note = TGF-Beta reverse primer
  source 1..20: mol_type = other DNA
gtcaatgtacagctgccgca

SEQ ID NO: 43 - DNA, 21 bp, synthetic construct
  misc_feature 1..21: note = YAP1 forward primer
  source 1..21: mol_type = other DNA
acccacagctcagcatcttcg

SEQ ID NO: 44 - DNA, 21 bp, synthetic construct
  misc_feature 1..21: note = YAP1 reverse primer
  source 1..21: mol_type = other DNA
tggcttgttcccatccatcag

(19) United States
(12) Patent Application Publication
(10) Pub. No.: US 2025/0114331 A1
(43) Pub. Date: Apr. 10, 2025

(54) CONCURRENT TARGETING OF ONCOGENIC PATHWAYS TO ENHANCE CHEMOTHERAPY AND IMMUNOTHERAPY

(71) Applicant: THE JOHNS HOPKINS UNIVERSITY, Baltimore, MD (US)

(72) Inventors: Mohammad O. Hoque, Towson, MD (US); Pritam Sadhukhan, Baltimore, MD (US)

(21) Appl. No.: 18/293,456

(22) PCT Filed: Aug. 1, 2022
(86) PCT No.: PCT/US2022/074397

§ 371 Date: Jan. 30, 2024

Related U.S. Application Data
(60) Provisional application No. 63/227,393, filed on Jul. 30, 2021.

(51) Int. Cl. A61K 31/409; A61K 31/255; A61K 45/06; A61P 35/00

(52) U.S. Cl. CPC A61K 31/409; A61K 31/255; A61K 45/06; A61P 35/00

(57) ABSTRACT

The present invention relates to the field of cancer. More specifically, the present invention provides compositions and methods for treating cancer by targeting YAP1 and STAT3 pathways. In a specific embodiment, the present invention provides a method for increasing sensitivity of cancer in a subject to chemotherapy and/or immunotherapy comprising the step of administering to the subject an effective amount of a YAP1 inhibitor and a STAT3 inhibitor.

### CROSS-REFERENCE TO RELATED APPLICATIONS

This application claims the benefit of U.S. Provisional Application No. 63/227,393, filed Jul. 30, 2021, which is incorporated herein by reference in its entirety

### GOVERNMENT SUPPORT CLAUSE

This invention was made with government support under grant no. CA208709 and grant no. CA206027, awarded by the National Institutes of Health. The government has certain rights in the invention.

### FIELD OF THE INVENTION

The present invention relates to the field of cancer. More specifically, the present invention provides compositions and methods for treating cancer by targeting YAP1 and STAT3 pathways to enhance chemotherapy and immunotherapy.

### REFERENCE TO AN ELECTRONIC SEQUENCE LISTING

The text of the computer readable sequence listing filed herewith, titled “P16935-02”, created Aug. 1, 2022, having a file size of 56,498 bytes, is hereby incorporated by reference in its entirety.

### BACKGROUND OF THE INVENTION

Resistance to cancer therapy remains a significant obstacle in treating patients with various solid malignancies. Exposure to current chemotherapeutics and targeted agents invariably leads to therapy resistance, heralding the need for novel agents. Cancer stem cells (CSCs)—a subpopulation of tumor cells with capacities for self-renewal and multi-lineage differentiation—represent a pool of therapeutically resistant cells. CSCs often share physical and molecular characteristics with the stem cell population of the human body. It remains challenging to selectively target CSCs in therapeutically resistant tumors. The generation of CSCs and induction of therapeutic resistance can be attributed to several deregulated critical growth regulatory signaling pathways. Beyond growth regulatory pathways, CSCs also change the tumor microenvironment and resist endogenous immune attack. Thus, CSCs can interfere with each stage of carcinogenesis from malignant transformation to the onset of metastasis to tumor recurrence. New strategies are needed for overcoming therapeutic resistance and achieving durable curative responses.

### SUMMARY OF THE INVENTION

The present invention is based, at least in part, on the present inventors discovery that combinatorial therapy consisting of targeting cancer stem cells regulating pathway and administering systemic therapy, such as chemotherapy and/or immunotherapy, might be an effective strategy to combat immune suppressive tumor microenvironment (TME) and therapeutic resistance. As described herein, the present invention provides compositions and methods for treating cancer using YAP1 and/or STAT3 inhibitors in combination with chemotherapy and/or immunotherapy.

In one aspect, the present invention increases sensitivity of the cancer to systemic therapy. In one embodiment, a method for treating cancer in a subject in need thereof comprises the step of administering to the subject an effective amount of a YAP1 inhibitor and a STAT3 inhibitor. In another embodiment, the subject is further treated with chemotherapy. In yet another embodiment, the subject is further treated with immunotherapy. In particular embodiments, the YAP1 inhibitor and STAT3 inhibitor increase sensitivity of the cancer in the subject to chemotherapy. In certain embodiments, the YAP1 inhibitor and STAT3 inhibitor increase sensitivity of the cancer in the subject to immunotherapy.

In a specific embodiment, the present invention provides a method for increasing sensitivity of cancer in a subject to chemotherapy and/or immunotherapy comprising the step of administering to the subject an effective amount of a YAP1 inhibitor and a STAT3 inhibitor. In another specific embodiment, the method further comprises the step of administering to the subject chemotherapy and/or immunotherapy. In certain embodiments, the immunotherapy comprises a checkpoint inhibitor including, but not limited to, a CTLA4 inhibitor, a PD-1 inhibitor and a PD-L1 inhibitor. In particular embodiments, the YAP1 inhibitor comprises verteporfin or a derivative thereof. In certain embodiments, the STAT3 inhibitor comprises S3I-201 or a derivative thereof. In specific embodiments, the cancer comprises lung, head and neck, and bladder cancers. In more specific embodiments, the cancer comprises lung adenocarcinoma or urothelial bladder cancer.

In particular embodiments, the cancer can comprise a cancer described on pages 14-15. In certain embodiments, the YAP1 inhibitor comprises one or more of the compounds described herein (e.g., on pages 19-23). In other embodiments, the STAT3 inhibitor comprises one or more of the compounds described herein (e.g., on pages 23-24). In further embodiments, the chemotherapy or chemotherapeutic agents comprises one or more of the compounds described herein (e.g., on pages 24-29. In specific embodiments, the immunotherapy or immunotherapeutic agent comprises one or more of the compounds described herein (e.g., on pages 29-32).

### BRIEF DESCRIPTION OF THE FIGURES

FIG. 1A-1E. Attenuation of YAP1 expression impairs cell proliferation and increases sensitivity to anticancer agents in lung adenocarcinoma. FIG. 1A: Western blotting confirmed YAP1 knockdown in H1299 and H1437 cells and YAP1 overexpression in A549 cells. FIG. 1B: Proliferation assay: YAP1-deficient cells (H1299 YAP1-sh and H1437 YAP1-sh) showed lower proliferative ratios than control cells, whereas YAP1-overexpressing cells (A549 YAP1-LV) proliferated more rapidly. FIG. 1C: Cells were treated with cisplatin (CDDP), and cell viability was recorded at 72 hours after treatment. H1299 and H1437 YAP1-sh cells showed greater sensitivity to cisplatin and YAP1-LV cells showed higher resistance, than their control counterparts. FIG. 1D: YAP1-deficient cells treated with cisplatin (2 or 5 mmol/L for 72 hours) showed greater percentages of cells undergoing apoptosis than control cells, whereas YAP1-LV cells showed substantially less apoptosis. FIG. 1E: Both YAP1-sh cell lines showed significantly suppressed tumor growth in athymic mice, whereas YAP1-LV cells formed tumors more aggressively than control cells (3 mice with tumors in both flanks per each group). Error bars, mean+SEM (*, P<0.05; **, P<0.001).

FIG. 2A-2G. YAP1 promotes STAT3 phosphorylation via IL6 upregulation. FIG. 2A: Immunoblotting shows YAP1, STAT3, and pSTAT3 expressions in YAP1-deficient (H1299 and H1437) and YAP1-overexpressing (A549) lung adenocarcinoma cells. YAP1 expression was positively associated with pSTAT3 expression. FIG. 2B: qRT-PCR data show IL6 mRNA expression to be positively associated with YAP1 expression in H1299, H1437, and A549 cell lines. FIG. 2C: Secretion of IL6 into media was significantly reduced in YAP1-sh cells, whereas higher levels were observed in YAP1-LV cells, as measured by ELISA in cell culture supernatant. Immunoblotting of pSTAT3 and b-actin in the respective cell lines is shown below the graph. FIG. 2D: Addition of recombinant human IL6 (0.1, 1, or 10 ng/mL) to H1299 and H1437 YAP1-sh cells increased pSTAT3 levels dose dependently. FIG. 2E: Blocking IL6 activity with IL6-neutralizing antibody (Ab, 1 mg/mL) inhibited pSTAT3 in a time-dependent manner without influencing total STAT3 expression in A549 YAP1-LV cells. FIG. 2F: ChIP-PCR assay with YAP1 antibody was conducted using H1299, H1437, and A549 cells. DNA isolation followed by PCR showed that YAP1 protein directly binds to the IL6 gene promoter region in three lung adenocarcinoma cells analyzed (white arrows). “Input” indicates DNA lysate before ChIP, which was diluted to 2% for PCR reactions. Histone H3 antibody and normal rabbit IgG served as positive and negative control, respectively. FIG. 2G: Schematic shows YAP1 binding to IL6 promoter and upregulating its transcription to induce STAT3 phosphorylation. Error bars, mean±SEM (*, P<0.05; **, P<0.001).

FIG. 3A-3C. Positive correlation between YAP1 and pSTAT3 expressions in human lung adenocarcinoma tissues. FIG. 3A: Representative images of YAP1 and pSTAT3 immunostaining in the TMA (scale bar, 100 mm). FIG. 3B: IHC results indicate a statistically significant correlation between YAP1 and pSTAT3 expressions (P<0.0001, ×2 test). FIG. 3C: YAP1 and pSTAT3 expressions were evaluated by immunoblotting in 13 lung adenocarcinoma PDXs. All YAP1-expressing PDXs (CTG0162, 0178, 0502, 0848, 1309, 1762, 2017, and 2708) also expressed pSTAT3.

FIG. 4A-4G. Combined genetic inhibition of YAP1 and STAT3 by inhibiting transcripts enhances cisplatin's cytotoxicity and attenuates malignant CSC-like features more than inhibition of YAP1 or STAT3 alone. FIG. 4A: Functional rescue of STAT3 in YAP1-sh cells by forced expression of STAT3 (STAT3-LV). FIG. 4B: YAP1-sh/STAT3-LV cells (H1299 and H1437) showed less sensitivity to cisplatin (CDDP) than YAP1-sh/STAT3-Ctrl cells, but more sensitivity than YAP1-Ctrl/STAT3-Ctrl cells. Cell viability was measured at 72 hours after 5 mmol/L cisplatin treatment. FIG. 4C: STAT3 inhibition by RNAi in YAP1-LV cells (A549). Immunoblotting shows lower expressions of pSTAT3 and STAT3 in YAP1-LV/STAT3-si cells. FIG. 4D: STAT3 inhibition restored cisplatin sensitivity as compared with YAP1-LV/STAT3-Ctrl cells. The YAP1-LV group was more resistant to cisplatin than were the YAP1-Ctrl/STAT3-Ctrl cells. FIG. 4E: Effects of dual genetic inhibition of YAP1 and STAT3 in H1299 and H1437 cell lines. Immunoblotting indicated that pSTAT3 and STAT3 expressions were inhibited by STAT3 siRNA transduction. FIG. 4F: YAP1-sh/STAT3-si cells showed the highest sensitivity to cisplatin compared with either target inhibition or control cells. FIG. 4G: Sphere formation assay: YAP1-sh/STAT3-si cells formed the fewest spheroids compared with the single-molecule inhibition and controls. Error bars, mean 1 SEM (*, P<0.05; **, P<0.001).

FIG. 5A-5B. Evaluation of verteporfin and S3I-201 as inhibitors of YAP1 and STAT3, respectively, in lung adenocarcinoma cells. FIG. 5A: Verteporfin suppressed YAP1 and STAT3 expressions in H1299 and H1437 cells in a concentration dependent manner. STAT3 monomer (black arrows) was decreased as verteporfin concentration was increased, whereas high molecular weight complexes (regions surrounded by circles) were increased, indicating oligomerization of STAT3. FIG. 5B: S3I-201 suppressed pSTAT3 in a dose-dependent manner.

FIG. 6A-6F. Therapeutic efficacy of verteporfin and S3I-201 in cell lines and patient-derived preclinical xenograft mouse models of human lung adenocarcinoma. FIG. 6A-6D: NSG mice were subcutaneously inoculated with lung adenocarcinoma cell lines (H1299 and H1437 cells) and PDXs that endogenously expressed YAP1 and pSTAT3. Animals were randomly assigned into different treatment groups (n=5). Treatments were started when tumors reached 200±50 mm³. FIG. 6A: Combined verteporfin+S3I-201 significantly inhibited tumor growth in an H1299 xenograft model. Verteporfin or S3I-201 treatment alone led to inhibited growth, but combined treatment had a greater inhibitory effect. FIG. 6B: In H1437 xenografts, only the combination of verteporfin+S3I-201 inhibited growth significantly more than controls, whereas either agent individually did not significantly affect tumor growth. FIGS. 6C and 6D: Two EGFR wild-type PDXs (CTG0162 and CTG0178) that expressed both YAP1 and pSTAT3 (FIG. 3C) were implanted. The “all” combination [cisplatin (CDDP)+gemcitabine (GEM)+verteporfin+S3I-201] dramatically impaired growth of both PDXs. Chemo+verteporfin also significantly retarded tumor growth until day 28 in CTG0162 (FIG. 6C) and day 34 in CTG0178 (FIG. 6D). However, after 4 to 5 weeks, tumors treated with chemo+verteporfin showed tumor regrowth. FIGS. 6E and 6F: Pharmacodynamic analysis of PDX tumors treated with verteporfin, S3I-201, and chemotherapy drugs. CTG0162 (FIG. 6E) and CTG0178 (FIG. 6F) tumors resected at 18 days after treatment (one sample per treatment; left). Both YAP1 and STAT3 were suppressed in the chemo+verteporfin and “all” treatment groups. Adding verteporfin to chemotherapy decreased NANOG and SOX2 expressions. Tumors resected at day 43 in CTG0162 (FIG. 6E) and day 46 in CTG0178 (FIG. 6F) were assessed (two samples per treatment; right). Expressions of YAP1 and STAT3 were higher in tumors treated with chemo+verteporfin than with the “all” group. Tumors from the “all” group had lower expression of NANOG in CTG0162, and both NANOG and SOX2 in CTG0178, than did the chemo+verteporfin group. Error bars, mean±SEM (*, P<0.05; **, P<0.001; N.S., not significant).

FIG. 7A-7C. FIG. 7A: YAP1 expression levels were decreased in H1299 and H1437 YAP1-knockdown cells and increased in A549 YAP1-overexpression cells. FIG. 7B: Immunoprecipitation with YAP1 antibody did not show direct binding between YAP1 and STAT3. “Input” indicates protein lysate without immunoprecipitation. IgG served as a negative control. FIG. 7C: mRNA expression levels of several cytokines and growth factors that can stimulate STAT phosphorylation. EGF, IFN-γ, IL-2, IL-4, IL-10 and TGF-β expression levels did not show any association with YAP1 expression in three cell lines. Error bar: mean±SEM. *P<0.05. N.S., not significant.

FIG. 8. Correlation between YAP1, IL-6, and STAT3 mRNA expressions in human 59 LUAD samples. There was a positive correlation between YAP1 and IL-6 mRNA expression levels, although no significant correlation was observed between YAP1 and STAT3 mRNA expression levels.

FIG. 9A-9E. Analysis of YAP1-STAT3 cross-talk and its implication for CSC function. FIG. 9A: Overexpression of lentiviral-based STAT3 cDNA in YAP1-deficient cells (YAP1-sh) restored proliferation defects to YAP1-sh/STAT3-Ctrl cells, although their growth ratios were still lower than for YAP1-Ctrl/STAT3-Ctrl cells. FIG. 9B: STAT3 inhibition in A549 YAP1-LV cells resulted in decreased proliferative ratio compared with YAP1-LV/STAT3-Ctrl cells. FIG. 9C: Cells with dual inhibition of YAP1 and STAT3 showed the lowest proliferative ratios compared with cells in which YAP1 or STAT3 alone was inhibited, or control cells. FIG. 9D: Pictures of spheroids. Dually inhibited cells formed the fewest and smallest spheroid cells. FIG. 9E: Transcript levels of CSC markers for NSCLC in spheroid cells and bulk parental cells. ABCG2, ALDH1A1, CD24, NANOG, OCT4 and SOX2 showed greater expression in the spheroid cells. Error bar: mean±SEM. *P<0.05, **P<0.001.

FIG. 10A-10B. Analysis of verteporfin and S3I-201 cytotoxicity in LUAD cells. FIG. 10A: IC₅₀s of verteporfin and S3I-201 in H1299 and H1437 cell lines were determined by cell viability assays. FIG. 10B: Isobologram analysis: IC₅₀ value of each drug was plotted as [(IC₅₀ of verteporfin), 0] and [0, (IC₅₀ of S3I-201)] in the graph; the line connecting these two points was set as the standardized line. Concentrations of S3I-201 that inhibited 50% of the cells were then determined in combination with 0.05, 0.10 or 0.15 μM of verteporfin. Because all plots were located under the standardized line, verteporfin and S3I-201 were shown to have a synergistic cytotoxic effect. The table shows verteporfin concentration (left row), S3I-201 concentration on the standardized line (middle row) and the actual IC₅₀ value of S3I-201 (right row).

FIG. 11A-11B. Efficacies of verteporfin and S3I-201 in vitro at RNA levels. FIG. 11A: Verteporfin suppressed mRNA expression of YAP1, and its downstream targets, CTGF and CYR61, as the drug concentration was increased. FIG. 11B: S3I-201 inhibited mRNA expressions of not only NRP1 and PROS1 (STAT3-targeting genes) but also STAT3. Error bars: mean±SEM. *P<0.05.

FIG. 12A-12G. YAP1 is potential driver candidate for UCB progression and sternness. FIG. 12A-12B: High throughput data obtained from public databases representing the progression free survival period in patients with high and low level of YAP1 expression; TCGA-bladder Cancer Cohort (BLCA)(FIG. 12A); UROMOL2021 (FIG. 12B). FIG. 12: Representative immunoblots showing YAP1 expression level in different parental (WT) and YAP1-sh clones of MB49 cells. FIG. 12D: Cell proliferation rate shown in different YAP1 clones. CT: Sh Control, Sh-Y74 and Sh-Y77: YAP1 knockdown clones. FIG. 12E: Sphere formation assay in YAP1 knockdown MB49 clones. FIG. 12F: Representative immunoblots showing the YAP1 expression in different wild type mouse bladder cancer cell line. MB49, UPPL595, BBN975. FIG. 12G: Representative micrographs for sphere formation assay in VP (1 μM) treated mouse bladder cancer cells. Data represent mean±SD. *p<0.05, Student's t-test.

FIG. 13A-13E. YAP1 drives bladder cancer progression in vivo. FIG. 13A: In vivo tumor growth curve (left) and tumor mass (right) of cell derived xenograft using MB49 YAP1 clones in C57bl/6 mice (n=8 per group). FIG. 13B: In vivo tumor growth curve (left) and tumor mass (right) of cell derived xenograft using MB49 YAP1 clones in immunocompromised NSG mice (n=3 per group). FIG. 13C-13E: In vivo tumor growth curve of cell derived xenograft using FIG. 13C WT MB49, FIG. 13. UPPL 595 and 13E. BBN 975 cells in C57bl/6 mice. Control animals were treated with DMSO, Treated animals are administered with Verteporfin 3 times a week, 50 mg/kg body weight (n=3). In the bottom the corresponding graphs represents the tumor mass. Data represent mean±SD. *p<0.05, Student's t-test.

FIG. 14. GSEA of RNA-seq data (BFTC 905 YAP1 Sh-ct versus BFTC 905 YAP1 Sh-C26) showing the top pathways that are suppressed in YAP1 KD cells compared with YAP1 ct cells (n=3).

FIG. 15A-15C. YAP1 induces IL-6 expression during UCB progression qPCR results of IL-6 expression in FIG. 15A MB49 YAP1 clones; FIG. 15B Verteporfin treated mouse bladder cancer cells (n=3); FIG. 15C xenografts developed from MB49 clones.

FIG. 16A-16F. YAP1 induces an immune suppressive tumor microenvironment. FIG. 16A: FACS analysis representing the infiltration of MDSCs, CD8 T cells and CD4 T cells in xenograft tumors, bone marrow, spleen and blood (n=8). FIG. 16B: Expression of YAP1 and YAP1 signature genes in MDSC high group of tumors analyzed from TCGA database, comprised of 407 bladder cancer samples. FIG. 16C: IHC showing the presence of MDSCs (Gr-1) and CD8 T cells in xenografts developed from MB49 YAP1 clones (n=3). FIG. 16D: IHC showing the presence of MDSCs (Gr-1) and CD8 T cells in xenografts developed from WT mouse UCB cells and mouse were administered with Verteporfin (50 mg/kg bw; 3 times a week) (n=3). FIG. 16E: FACS analysis of the expression of CD107 and IFNγ in xenograft tumor from MB49 YAP1 clones, representing the cytotoxic activity CD8 T cells (n=8). FIG. 16F: CDX model developed with MB49 WT cells and treated with anti-MDSC antibody. The left panel represents the tumor growth rate and tumor mass is shown in the right (n=3). Data represent mean±SD. *p<0.05, **p<0.01 Student's t-test.

FIG. 17A-17D. YAP1 potentially modulates the activity of MDSCs and Macrophages in the xenograft tumor. FIG. 17A: MDSC migration assay with MDSCs from MB49 xenografts (from YAP1 clones) and cultured with conditioned media from MB49 YAP1 clones. FIG. 17B: Macrophage migration assay with primary macrophages from WT C57bl/6 animals (from YAP1 clones) and cultured with conditioned media from MB49 YAP1 clones. FIG. 17C: qPCR analysis in macrophage polarization markers, RAW cell line cultured with conditioned media from MB49 YAP1 clones. FIG. 17D: qPCR analysis of various chemokines in MB49 YAP1 clones and xenograft developed from MB49 YAP1 clones.

FIG. 18A-18G. YAP1 potentially regulates the expression of CXCR2 and associated ligands. FIG. 18A-18D: qPCR assay showing the expression of CXCR2 associated ligands in human bladder cancer cell lines (YAP1 sh-clones). FIG. 18A, BFTC 905 cells; FIG. 18B, T24 cells; FIG. 18C, BFTC 909 cells; FIG. 18D, UMUC3 cells. FIG. 18E-18F: Correlation between CXCR2 associated ligands and YAP1 in primary UCB cohort and TCGA cohort. FIG. 18G: FACS analysis showing CXCR2 expression in the tumor, blood and spleen of MB49 YAP1 clones bearing xenografts.

FIG. 19A-19D. YAP1 influences the accumulation of lipid droplets in cancer cells. FIG. 19A: Fluorescence images showing lipid droplets accumulation in MB-49 YAP1 clones. FIG. 19B-19C: Fluorimetric quantification of the lipid droplet accumulation in MB49 YAP1 clones. FIG. 19D: Fluorimetric quantification of the lipid droplet accumulation in WT mouse UCB cells upon verteporfin treatment.

FIG. 20A-20E. YAP knockdown Tumors Stimulates Host Adaptive Immunity. FIG. 20A: Tumor growth curve of MB49 WT or YAP1 clones. MB49 WT or YAP1 clones were injected into C57BL/6 mice, and tumor growth was monitored after the indicated times. For coinjection experiments, MB49 WT or YAP1 clones were injected into opposite flanks in the same mouse (right panel). FIG. 20B-20C: Tumor mass and representative images from the co-injection study. FIG. 20D: IHC showing the expression of MDSCs (Gr-1) and CD8 T cells in xenografts developed from MB49 WT tumors and co injected with the MB49 YAP1 clones in the different flank of the same mouse. FIG. 20E: IL-6 expression in xenografts developed from MB49 WT tumors and co injected with the MB49 YAP1 clones.

FIG. 21. EVs isolated from culture supernatants of equal numbers of MB49 WT or YAP1 clones and were subjected to nanoparticle tracking analysis (NanoSight) to quantify the number and size distribution.

FIG. 22A-22M. YAP1 is potential driver candidate for UCB progression and stemness. FIG. 22A-22B: High throughput data obtained from public databases representing the overall survival (FIG. 22A) and disease-free survival (FIG. 22B) in patients with high (top 25%) and low level (Least 25%) of YAP1 expression in TCGA-BLCA database. FIG. 22C: Immunoblots showing YAP1 expression level in different parental (WT) and YAP1-sh clones of MB49 cells. FIG. 22D: Cell proliferation rate shown in different YAP1 clones. CT: Sh Control, Sh-Y74 and Sh-Y77: YAP1 knockdown clones. FIG. 22E: Sphere formation assay in YAP1 knockdown MB49 clones. FIG. 22F: Immunoblots showing the YAP1 expression in different wild type mouse bladder cancer cell line. MB49, UPPL595, BBN975. FIG. 22G: Sphere formation assay in VP (1 μM) treated mouse bladder cancer cells. FIG. 22L: Comparison of wound healing potential in VP treated UPPL 595 cells compared to control cells (n=3). FIG. 22M: Comparison of wound healing potential in siYAP1 treated UPPL 595 cells compared to control cells (n=3).

FIG. 23A-23G. YAP1 drives bladder cancer progression in vivo. FIG. 23A: In vivo tumor growth curve (left) and tumor mass (right) of cell derived xenograft using MB49 YAP1 clones in C57bl/6 mice. FIG. 23B: In vivo tumor growth curve (left) and tumor mass (right) of cell derived xenograft using MB49 YAP1 clones in immunocompromised NSG mice. FIG. 23C-23E: In vivo tumor growth curve of cell derived xenograft using WT MB49, UPPL 595 and BBN 975 cells in C57bl/6 mice. Control animals were treated with DMSO, Treated animals are administered with Verteporfin 3 times a week, 50 mg/kg body weight. FIG. 23F: Immunoblots showing YAP1 expression level in CDX from C57Bl/6 animals, treated with verterporfin.

FIG. 24A-24E. FIG. 24A: Graphical representation of top upregulated pathways in MB49 YAP1 Sh-Ct (YAP1 expressing) cells compared to MB49 YAP1 Sh-Y74 (YAP1 KD) cells. FIG. 24B: Molecular network showing top down regulated pathways in MB49 YAP1 Sh-Ct (YAP1 expressing) cells compared to MB49 YAP1 Sh-Y74 (YAP1 KD) cells. FIG. 24C: qPCR analysis of the key immunoregulatory genes in MB49 YAP1 KD clones. FIG. 24D: qPCR analysis of the key MHC molecules (H2-K, CD80 and H2-Ab) in YAP1 KD clones. FIG. 24E: qPCR analysis of the key MHC molecules (H2-K, CD80 and H2-Ab) in YAP1 expressing mouse UCB cell lines.

FIG. 25A-25K. YAP1 potentially induces an immune suppressive tumor microenvironment. FIG. 25A: Expression of YAP1 and YAP1 signature genes in MDSC high group of tumors analysed from TCGA database, comprised of 407 bladder cancer samples. FIG. 25B: FACS analysis representing the infiltration of MDSCs, CD8 T cells and CD4 T cells in xenograft tumors, bone marrow, spleen and blood (n=8). FIG. 25C: FACS analysis of the expression of CD107 and IFNγ in xenograft tumor from MB49 YAP1 clones, representing the cytotoxic activity CD8 T cells (n=8). FIG. 25D: Co-culture cytotoxicity assay of CD8+ T cells and cancer cells measured by quantifying the released LDH in the culture media. FIG. 25E: IHC showing the presence of MDSCs (Gr-1) and CD8 T cells in xenografts developed from MB49 YAP1 clones (n=3). FIG. 25F: IHC showing the presence of MDSCs (Gr-1) and CD8 T cells in xenografts developed from WT mouse UCB cells and mouse were administered with Verteporfin (50 mg/kg bw; 3 times a week) (n=3). FIG. 25G: CDX model developed with MB49 WT cells and treated with anti-MDSC antibody. The left panel represents the tumor growth rate and tumor mass is shown in the right (n=3). data represent mean±SD. *p<0.05, **p<0.01 Student's t test.

FIG. 26A-26N. YAP1 potentially modulates the activity of MDSCs and Macrophages in the xenograft tumor. FIG. 26A: Macrophage migration assay with primary macrophages from WT C57bl/6 animals (from YAP1 clones) and cultured with conditioned media from MB49 YAP1 clones. FIG. 26B: MDSC migration assay with MDSCs from MB49 xenografts (from YAP1 clones) and cultured with conditioned media from MB49 YAP1 clones. FIG. 26C: IHC showing the presence of macrophages (f4/80) in xenografts developed from MB49 YAP1 clones (n=3). FIG. 26D: qPCR analysis in macrophage polarization markers in RAW 264.7 cell line cultured with conditioned media from MB49 YAP1 clones. FIG. 26E: ELISA showing the level of different cytokines released from macrophages incubated with the CM of YAP1 KD clones. FIG. 26F: Griess assay showing the level of NO in the culture media of macrophages incubated with the CM of YAP1 KD clones. FIG. 26G: qPCR analysis of various chemokines in MB49 YAP1 clones. FIG. 26H: qPCR analysis of various chemokines in MB49 xenograft developed from MB49 YAP1 clones. FIG. 26I: FACS analysis showing CXCR2 expression in the tumor, blood and spleen of MB49 YAP1 clones bearing xenografts. FIG. 26J-26M: qPCR assay showing the expression of CXCR2 associated ligands in human bladder cancer cell lines (YAP1 sh-clones). FIG. 26G, BFTC 905 cells; FIG. 26H, T24 cells; FIG. 26I, BFTC 909 cells; FIG. 26J, UMUC3 cells.

FIG. 27A-M. YAP1 activates IL-6/STAT3 pathway during UCB progression. FIG. 27A: Analysis of IMVIGOR210 database showing the expression level of IL-6 and YAP1 in the cohort. The patient data were divided into four groups: CR, complete response; PR, partial response; SD, Stable disease; PD, progressive disease. FIG. 27B: Expression pattern of YAP1 in IMVIGOR210 database among immunotherapy response group (CR and PR) as compared to non-responsive group (SD and PD). FIG. 27C: Expression pattern of IL-6 in IMVIGOR210 database among immunotherapy different groups, (CR, PR, SD and PD). FIG. 27D-27H: Clinical significance of IL-6 expression in cancer progression and immune response. FIG. 27I: Immuno blot showing the expression of IL-6 in VP treated CDX bearing C57bl/6 mice. CDX were generated from WT cells of MB49, UPPL1595 and BBN975 cells. FIG. 27J-27M: qPCR results of IL-6 expression in e, MB49 YAP1 clones; FIG. 27F, Verteporfin treated mouse bladder cancer cells; FIG. 27G, xenografts developed from MB49 clones; FIG. 27H, Xenografts developed from WT mouse bladder cancer cells.

FIG. 28A-28E. STAT3 inhibition mimics the antitumor activity of YAP1 attenuation. FIG. 28A: Tumor growth curve of MB49 WT cells in C57BL/6 animals treated with STAT3 inhibitor S3I-201. FIG. 28B: Tumor mass representing the tumors from same animals of (FIG. 28A). FIG. 28C: q-PCR analysis showing the expression of different CSC markers (FIG. 28E) in the tumor tissue. FIG. 28D: q-PCR analysis showing the expression of different CXCR1/CXCR2 associated ligands (FIG. 28D) and CSC markers (FIG. 28E) in the tumor tissue. FIG. 28E: IHC showing the infiltration of MDSCs (Gr-1) and CD8+ T cells in the TME.

FIG. 29A-29J. YAP1 influences the accumulation of lipid droplets in cancer cells. FIG. 29A: Micrographs (10×) showing the lipid droplets in MB-49 YAP1 KD clones. FIG. 29B: Quantification of LD accumulation in MB-49 YAP1 KD clones by fluorescent spectroscopy. FIG. 29C-29F: Quantification of LD accumulation in MB49 YAP1 KD clones exposed to exogenous Oleic acid (FIG. 29C); YAP1 expressing mouse UCB cell clines (MB49, UPPL1595 and BBN975) (FIG. 29D); human UCB cell lines (YAP1 was KD in BFTC 905 and T24 cell line; YAP1 was OE in BFTC909 cell line) (FIG. 29E); Vetrporfin treated WT human UCB cell lines (FIG. 29F). FIG. 29G-29J: Quantification of L-Lactate in MB49 YAP1 KD clones (FIG. 29G); YAP1 expressing mouse UCB cell clines (MB49, UPPL1595 and BBN975) (FIG. 29H); human UCB cell lines (YAP1 was KD in BFTC 905 and T24 cell line; YAP1 was OE in BFTC909 cell line) (FIG. 29I); Vetrporfin treated WT human UCB cell lines (FIG. 29J). Data represent mean±SD. **p<0.01, ***p<0.001, ****p<0.0001.

FIG. 30A-30I. YAP knockdown Tumors Stimulates Host Adaptive Immunity. FIG. 30A: A schematic showing the cell injection pattern in the mice. WT: mice were injected with WT cells in both the flanks; Sh-74: mice were injected with MB49 YAP1sh-Y74 cells in both the flanks; Sh-77: mice were injected with MB49 YAP1 sh-Y77 cells in both the flanks; WT-[with Sh74]: mice were injected with WT cells in the left flank and MB49 YAP1sh-Y74 cells in the right flank; WT-[with Sh77]: mice were injected with WT cells in the left flank and MB49 YAP1sh-Y77 cells in the night flank. FIG. 30B: Tumor growth curve of MB49 WT or YAP1 clones. MB49 WT or YAP1 clones were injected into C57BL/6 mice, and tumor growth was monitored after the indicated times. FIG. 30C-30D: Tumor mass and representative tumor images from the co-injection study. FIG. 30E: IHC showing the expression of MDSCs (Gr-1) and CD8 T cells in xenogarfts developed from MB49 WT tumors and co injected with the MB49 YAP1 clones in the different flank of the same mouse. FIG. 30F: IL-6 expression in xenogarfts developed from MB49 WT tumors and co injected with the MB49 YAP1 clones. FIG. 30G: MB49 YAP1 KD cells were cultured in exosome depleted condition and after extracellular vesicles were isolated and subsequently quantified in a nanosight. FIG. 30H: Total EV proteins were isolated from 1 million cells and total protein was quantified using BCA method. FIG. 30I: Isolated EV were exposed to RAW264.7 cell lines and macrophage polarization markers were quantified using qRT PCR. Data represent mean±SD. **p<0.01, ***p<0.001, ****p<0.0001.

FIG. 31A-31I. YAP1 showed synergistic anti-tumor efficacy in combination with anti-PD-L1. FIG. 31A-31B: Tumor growth curve of MB49 YAP1 clones (FIG. 31A)/MB49 WT cells (FIG. 31B). MB49 YAP1 clones/MB49 WT cells were subcutaneously injected into C57BL/6 mice and treated with VP and anti-PD-L1. Tumor growth was monitored at the indicated times. FIG. 31C-31D: IHC showing the expression of MDSCs (Gr-1) and CD8 T cells in xenogarfts developed from MB49 WT tumors. FIG. 31E-31H: qPCR analysis from the xenograft tumors with gene primers specific for CSC markers (FIG. 31E), different tumor promoting CXCLs (FIG. 31F), imunogenecity markers (FIG. 31G), various key immune regulatory molecules (FIG. 31H). FIG. 31I: Tumor growth curve of MB49 WT cells in control animals and mice previously treated with VP+anti-PD-L1.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the articles “a” and “an” are used to refer to one or to more than one (i.e., to at least one) of the grammatical object of the article. By way of example, “an element” means one element or more than one element.

As used herein, “about,” when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. Unless specifically stated or obvious from context, as used herein, the term “about” is understood as within a range of normal tolerance in the art, for example within 2 standard deviations of the mean. About can be understood as being within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term “about.”

“Optional” or “optionally” means that the subsequently described event or circumstance can or cannot occur, and that the description includes instances where the event or circumstance occurs and instances where it does not.

“Agent” refers to all materials that may be used as or in pharmaceutical compositions, or that may be compounds such as small synthetic or naturally derived organic compounds, nucleic acids, polypeptides, antibodies, fragments, isoforms, variants, or other materials that may be used independently for such purposes, all in accordance with the present invention.

“Antagonist” refers to an agent that down-regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down-regulates expression of a gene or which reduces the amount of expressed protein present. The term “inhibitor” is synonymous with the term antagonist.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

A subject according to any of the methods described herein can have a “cancer” that includes, without limitation, lung cancer (e.g., lung adenocarcinoma, small cell lung carcinoma or non-small cell lung carcinoma), papillary thyroid cancer, medullary thyroid cancer, differentiated thyroid cancer, recurrent thyroid cancer, refractory differentiated thyroid cancer, lung adenocarcinoma, bronchioles lung cell carcinoma, multiple endocrine neoplasia type 2A or 2B (MEN2A or MEN2B, respectively), pheochromocytoma, parathyroid hyperplasia, breast cancer, colorectal cancer (e.g., metastatic colorectal cancer), papillary renal cell carcinoma, ganglioneuromatosis of the gastroenteric mucosa, inflammatory myofibroblastic tumor, or cervical cancer, acute lymphoblastic leukemia (ALL), acute myeloid leukemia (AML), cancer in adolescents, adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, unknown primary carcinoma, cardiac tumors, cervical cancer, childhood cancers, chordoma, chronic lymphocytic leukemia (CLL), chronic myelogenous leukemia (CML), chronic myeloproliferative neoplasms, colon cancer, colorectal cancer, craniopharyngioma, cutaneous T-cell lymphoma, bile duct cancer, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, extrahepatic bile duct cancer, eye cancer, fallopian tube cancer, fibrous histiocytoma of bone, gallbladder cancer, gastric cancer, gastrointestinal carcinoid tumor, gastrointestinal stromal tumors (GIST), germ cell tumor, gestational trophoblastic disease, glioma, hairy cell tumor, hairy cell leukemia, head and neck cancer, heart cancer, hepatocellular cancer, histiocytosis, Hodgkin's lymphoma, hypopharyngeal cancer, intraocular melanoma, islet cell tumors, pancreatic neuroendocrine tumors, Kaposi sarcoma, kidney cancer, Langerhans cell histiocytosis, laryngeal cancer, leukemia, lip and oral cavity cancer, liver cancer, lung cancer, lymphoma, macroglobulinemia, malignant fibrous histiocytoma of bone, osteocarcinoma, melanoma, Merkel cell carcinoma, mesothelioma, metastatic squamous neck cancer, midline tract carcinoma, mouth cancer, multiple endocrine neoplasia syndromes, multiple myeloma, mycosis fungoides, myelodysplastic syndromes, myelodysplastic/myeloproliferative neoplasms, myelogenous leukemia, myeloid leukemia, multiple myeloma, myeloproliferative neoplasms, nasal cavity and paranasal sinus cancer, nasopharyngeal cancer, neuroblastoma, non-Hodgkin's lymphoma, non-small cell lung cancer, oral cancer, oral cavity cancer, lip cancer, oropharyngeal cancer, osteosarcoma, ovarian cancer, pancreatic cancer, papillomatosis, paraganglioma, paranasal sinus and nasal cavity cancer, parathyroid cancer, penile cancer, pharyngeal cancer, pheochromosytoma, pituitary cancer, plasma cell neoplasm, pleuropulmonary blastoma, pregnancy and breast cancer, primary central nervous system lymphoma, primary peritoneal cancer, prostate cancer, rectal cancer, renal cell cancer, retinoblastoma, rhabdomyosarcoma, salivary gland cancer, sarcoma, Sezary syndrome, skin cancer, small cell lung cancer, small intestine cancer, soft tissue sarcoma, squamous cell carcinoma, squamous neck cancer, stomach cancer, T-cell lymphoma, testicular cancer, throat cancer, thymoma and thymic carcinoma, thyroid cancer, transitional cell cancer of the renal pelvis and ureter, unknown primary carcinoma, urethral cancer, uterine cancer, uterine sarcoma, vaginal cancer, vulvar cancer, Waldenstrom Macroglobulinemia, and Wilms' tumor.

In some embodiments of any of the methods described herein, the subject has lung adenocarcinoma, non-small cell lung cancer, melanoma, ovarian cancer, colorectal cancer, breast cancer and prostate cancer.

In some embodiments of any of the methods described herein, the subject has a head and neck cancer, a central nervous system cancer, a lung cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a sarcoma, a kidney cancer, a bladder cancer, an uterine cancer, a cervical cancer, and a prostate cancer.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

A “small molecule” refers to a composition that has a molecular weight of less than 3 about kilodaltons (kDa), less than about 1.5 kilodaltons, or less than about 1 kilodalton. Small molecules may be nucleic acids, peptides, polypeptides, peptidomimetics, carbohydrates, lipids or other organic (carbon-containing) or inorganic molecules. A “small organic molecule” is an organic compound (or organic compound complexed with an inorganic compound (e.g., metal)) that has a molecular weight of less than about 3 kilodaltons, less than about 1.5 kilodaltons, or less than about 1 kDa.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., a YAP1 inhibitor, a STAT3 inhibitor, a chemotherapeutic agent and/or immunotherapy. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of treating solid tumors in patients.

As used herein, the term “effective,” means adequate to accomplish a desired, expected, or intended result. More particularly, an “effective amount” or a “therapeutically effective amount” is used interchangeably and refers to an amount of, for example, a YAP1 inhibitor, a STAT3 inhibitor, a chemotherapeutic agent and/or immunotherapy necessary to provide the desired “treatment” (defined herein) or therapeutic effect, e.g., an amount that is effective to prevent, alleviate, treat or ameliorate symptoms of a disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The term “combination” refers to two or more therapeutic agents to treat a condition or disorder described herein. Such combination of therapeutic agents may be in the form of a single pill, capsule, or intravenous solution. However, the term “combination” also encompasses the situation when the two or more therapeutic agents are in separate pills, capsules, syringes or intravenous solutions. Likewise, the term “combination therapy” refers to the administration of two or more therapeutic agents to treat a therapeutic condition or disorder described herein. Such administration encompasses co-administration of these therapeutic agents in a substantially simultaneous manner, such as in a single capsule having a fixed ratio of active ingredients or in multiple, or in separate containers (e.g., pills, capsules, etc.) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The terms “co-administration” and “in combination with” include the administration of two or more therapeutic agents simultaneously, concurrently or sequentially within no specific time limits unless otherwise indicated. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., without limitation, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The term “tumor” refers to all neoplastic cell growth and proliferation, whether malignant or benign, and all pre-cancerous and cancerous cells and tissues. As used herein, the term “neoplastic” refers to any form of dysregulated or unregulated cell growth, whether malignant or benign, resulting in abnormal tissue growth. Thus, “neoplastic cells” include malignant and benign cells having dysregulated or unregulated cell growth.

The term “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site of one organ, or portion of the body, to another organ, or portion of the body, or in an in vitro assay system. Each carrier must be “acceptable” in the sense of being compatible with the other ingredients of the formulation and not injurious to a subject to whom it is administered. Nor should an acceptable carrier alter the specific activity of the subject compounds.

The term “pharmaceutically acceptable” refers to molecular entities and compositions that are physiologically tolerable and do not typically produce an allergic or similar untoward reaction, such as gastric upset, dizziness and the like, when administered to a human.

The term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

The term “prodrug” means a derivative of a compound that can hydrolyze, oxidize, or otherwise react under biological conditions (in vitro or in vivo) to provide the compound. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

The term “unit-dosage form” refers to a physically discrete unit suitable for administration to a human or animal subject, and packaged individually as is known in the art. Each unit-dose contains a predetermined quantity of an active ingredient(s) sufficient to produce the desired therapeutic effect, in association with the required pharmaceutical carriers or excipients. A unit-dosage form may be administered in fractions or multiples thereof. Examples of a unit-dosage form include an ampoule, syringe, and individually packaged tablet and capsule.

The term “multiple-dosage form” is a plurality of identical unit-dosage forms packaged in a single container to be administered in segregated unit-dosage form. Examples of a multiple-dosage form include a vial, bottle of tablets or capsules, or bottle of pints or gallons.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

As used herein, and unless otherwise specified, the terms “composition,” “formulation,” and “dosage form” are intended to encompass products comprising the specified ingredient(s) (in the specified amounts, if indicated), as well as any product(s) which result, directly or indirectly, from combination of the specified ingredient(s) in the specified amount(s).

### II. YAP1 Inhibitors

As used herein, “YAP1” (yes-associated protein 1), also known as YAP or YAP65, is a protein that acts as a transcriptional regulator by activating the transcription of genes involved in cell proliferation and suppressing apoptotic genes. YAP1 is inhibited in the Hippo signaling pathway, a pathway that may be involved in the cellular control of organ size and tumor suppression. YAP1 was first identified by virtue of its ability to associate with the SH3 domain of Yes and Src protein tyrosine kinases, and it is an oncogene.

In particular embodiments, a YAP1 inhibitor comprises verteporfin. As used herein, “verteporfin” refers to a compound having IUPAC name of (3-[(23S,24R)-14-ethenyl-5-(3-methoxy-3-oxopropyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoic acid). As a benzoporphyrin derivative, verteporfin has a trade name of Visudyne, and it is a medication used as a photosensitizer for photodynamic therapy to eliminate the abnormal blood vessels in the eye associated with conditions such as the wet form of macular degeneration.

In some embodiments, the YAP1 inhibitor includes a verteporfin derivative. In some embodiments, the YAP1 inhibitor includes at least one of the compounds of T1-T30 described in U.S. Patent Application Publication No. 20190298694:
T1 ((2S)-2-[3-[(23S,24R)-14-ethenyl-5-(3-methoxy-3-oxopropyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]butanedioic acid)
T2 ((2S)-2-[3-[(23S,24R)-14-ethenyl-5-(2-carboxyethyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]butanedioic acid)
T3 ((2S)-2-[3-[(23S,24R)-14-ethenyl-5-[3-[[(1S)-1,2-dicarboxyethyl]amino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]butanedioic acid)
T4((2S)-2-[3-[3-[(23S,24R)-14-ethenyl-5-(3-methoxy-3-oxopropyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]propanoylamino]butanedioic acid).
T5 ((2S)-2-amino-6-[3-[(23S,24R)-14-ethenyl-5-(3-methoxy-3-oxopropyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]hexanoic acid)
T6 ((2S)-2-amino-6-[3-[(23S,24R)-14-ethenyl-5-(2-carboxyethyl)-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]hexanoic acid)
T7 ((2S)-2-amino-6-[3-[(23S,24R)-14-ethenyl-5-[3-[[(5S)-5-amino-5-carboxy-pentyl]amino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoylamino]hexanoic acid)
T8 (Methyl 3-[(23S,24R)-14-ethenyl-9-[3-[2-[2-(2-methoxyethoxy)ethoxy]ethoxy]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-5-yl]propanoate)
T9 (2-[2-(2-methoxyethoxy)ethoxy]ethyl 3-[(23S,24R)-14-ethenyl-5-[3-[2-[2-(2-methoxyethoxy)ethoxy]ethoxy]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanoate)
T10 (Methyl 3-[(23S,24R)-14-ethenyl-9-[3-[2-[2-[2-[2-[2-[2-(2-methoxyethoxy)ethoxy]ethoxy]ethoxy]ethoxy]ethoxy]ethoxy]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-5-yl]propanoate)
T11 (Methyl 3-[(23S,24R)-14-ethenyl-9-[3-[2-(dimethylamino)ethylamino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-5-yl]propanoate)
T12 (3-[(23S,24R)-14-ethenyl-9-[3-[2-(dimethylamino)ethylamino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-5-yl]propanoic acid)
T13 (N-[2-(dimethylamino)ethyl]-3-[(23S,24R)-14-ethenyl-5-[3-[2-(dimethylamino)ethylamino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]propanamide)
T14 (Methyl 3-[(23S,24R)-14-ethenyl-9-[3-[2-(dimethylamino)ethyl-methyl-amino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-5-yl]propanoate)
T15 (N-[2-(dimethylamino)ethyl]-3-[(23S,24R)-14-ethenyl-5-[3-[2-(dimethylamino)ethyl-methyl-amino]-3-oxo-propyl]-22,23-bis(methoxycarbonyl)-4,10,15,24-tetramethyl-25,26,27,28-tetraazahexacyclo[16.6.1.13,6.18,11.113,16.019,24]octacosa-1,3,5,7,9,11(27),12,14,16,18(25),19,21-dodecaen-9-yl]-N-methyl-propanamide)
T16 ((3-(7-(3-methoxy-3-oxopropyl)-2,8,12,17-tetramethyl-13,18-divinyl-7H,8H-porphyrin-3-yl)propanoyl)-L-aspartic acid)
T17 ((3-(7-(2-carboxyethyl)-2,8,12,17-tetramethyl-13,18-divinyl-7H,8H-porphyrin-3-yl)propanoyl)-L-aspartic acid)
T18 ((3-(7-(5,6-dicarboxy-3-oxohexyl)-2,8,12,17-tetramethyl-13,18-divinyl-7H,8H-porphyrin-3-yl)propanoyl)-L-aspartic acid)
T19 ((3-(3-(7-(3-methoxy-3-oxopropyl)-2,8,12,17-tetramethyl-13,18-divinyl-7H,8H-porphyrin-3-yl)propanamido)propanoyl)-L-aspartic acid)
T20 (N6-(3-(7-(3-methoxy-3-oxopropyl)-2,8,12,17-tetramethyl-13,18-divinyl-7H,8H-porphyrin-3-yl)propanoyl)-L-lysine)
T21 (N6-(3-(7-(2-carboxyethyl)-2,8,12,17-tetramethyl-13,18-divinyl-7H,8H-porphyrin-3-yl)propanoyl)-L-lysine)
T22 ((2S,2'S)-6,6′-((3,3′-(2,8,12,17-tetramethyl-13,18-divinyl-7H,8H-porphyrin-3,7-diyl)bis(propanoyl))bis(azanediyl))bis(2-aminohexanoic acid))
T23 (2-(2-(2-methoxyethoxy)ethoxy)ethyl 3-(7-(3-methoxy-3-oxopropyl)-2,8,12,17-tetramethyl-13,18-divinyl-7H,8H-porphyrin-3-yl)propanoate)
T24 (bis(2-(2-(2-methoxyethoxy)ethoxy)ethyl) 3,3′-(2,8,12,17-tetramethyl-13,18-divinyl-7H,8H-porphyrin-3,7-diyl)dipropionate)
T25 (2,5,8,11,14,17,20-heptaoxadocosan-22-yl 3-(7-(3-methoxy-3-oxopropyl)-2,8,12,17-tetramethyl-13,18-divinyl-7H,8H-porphyrin-3-yl)propanoate)
T26 (methyl 3-(3-(3-((2-(dimethylamino)ethyl)amino)-3-oxopropyl)-2,8,12,17-tetramethyl-13,18-divinyl-7H,8H-porphyrin-7-yl) propanoate)
T27 (3-(3-(3-((2-(dimethylamino)ethyl)amino)-3-oxopropyl)-2,8,12,17-tetramethyl-13,18-divinyl-7H,8H-porphyrin-8-yl)propanoic acid)
T28 (3,3′-(2,8,12,17-tetramethyl-13,18-divinyl-7H,8H-porphyrin-3,7-diyl)bis(N-(2-(dimethylamino)ethyl) propanamide))
T29 (methyl 3-(3-(3-((2-(dimethylamino)ethyl)(methyl)amino)-3-oxopropyl)-2,8,12,17-tetramethyl-13,18-divinyl-7H,8H-porphyrin-7-yl)propanoate)
T30 (N-(2-(dimethylamino)ethyl)-3-(7-(3-((2-(dimethylamino) ethyl)amino)-3-oxopropyl)-2,8,12,17-tetramethyl-13,18-divinyl-7H,8H-porphyrin-3-yl)-N-methylpropanamide)

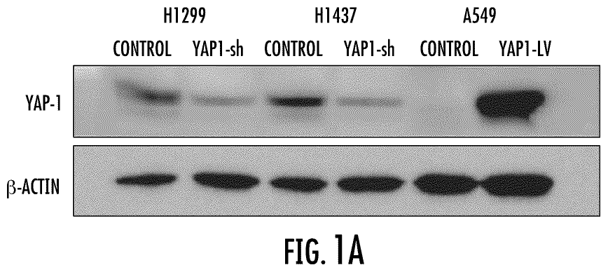

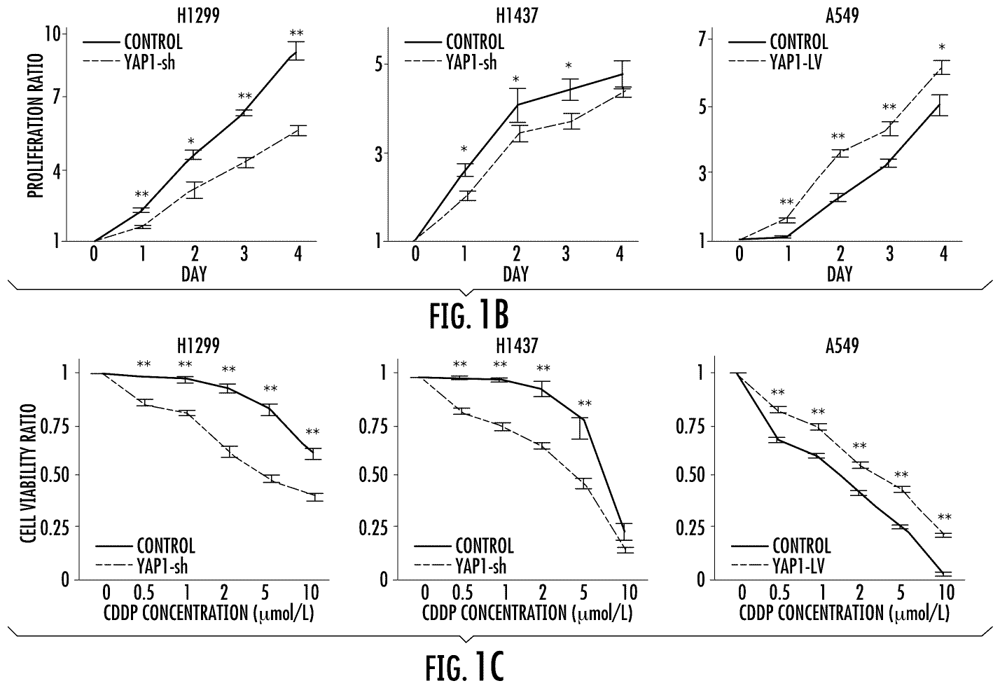

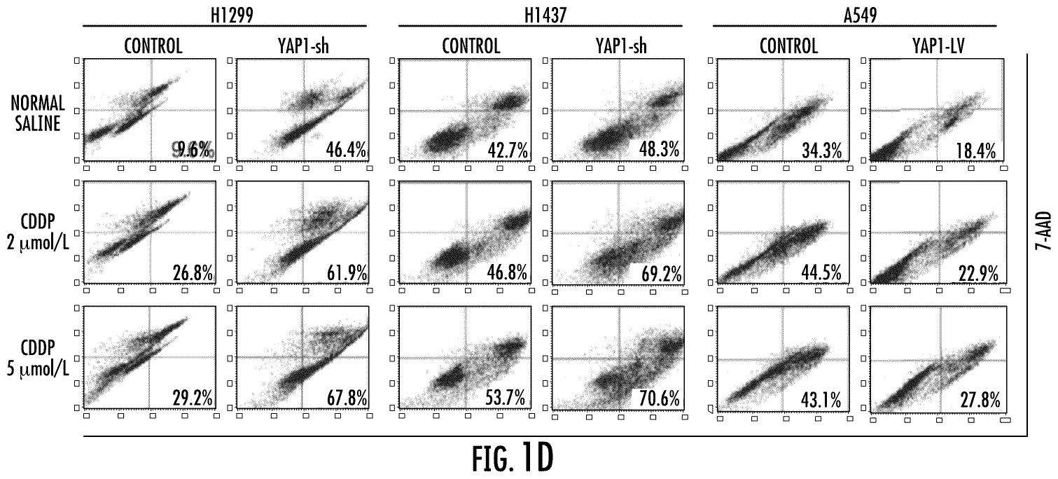

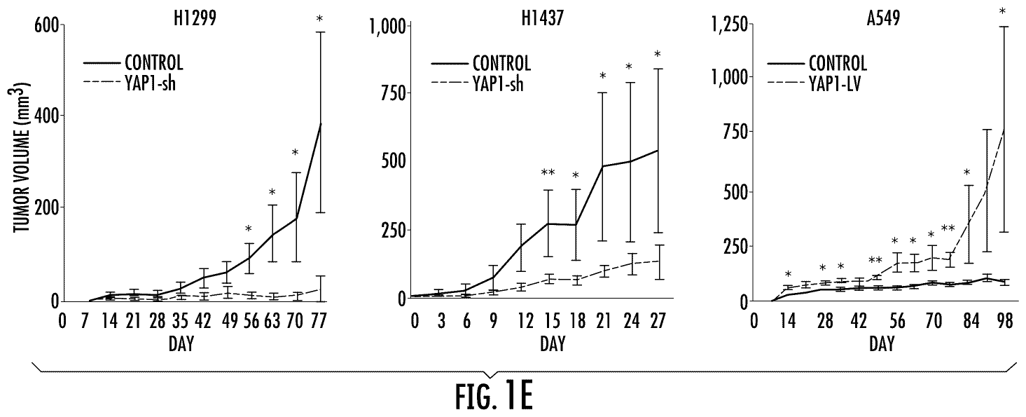

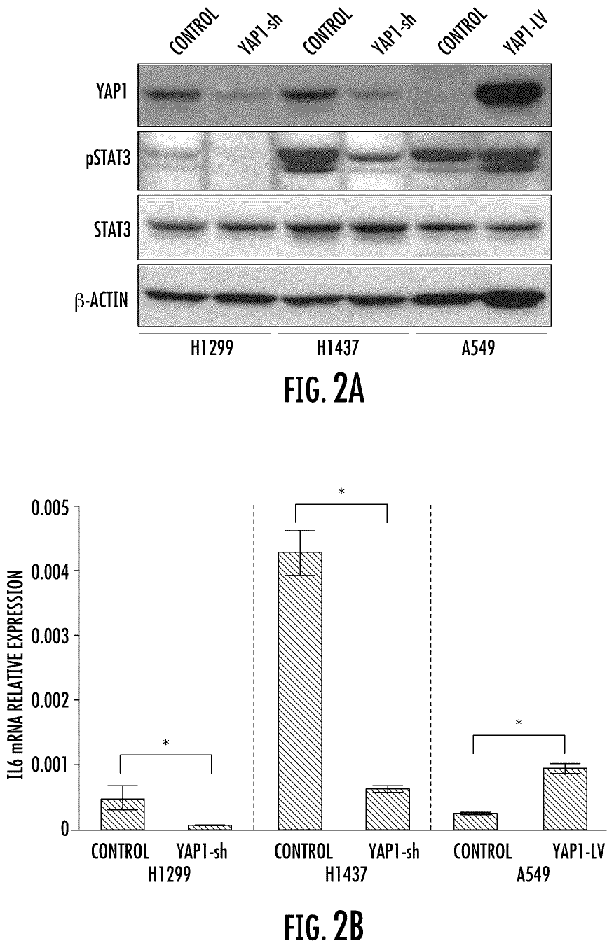

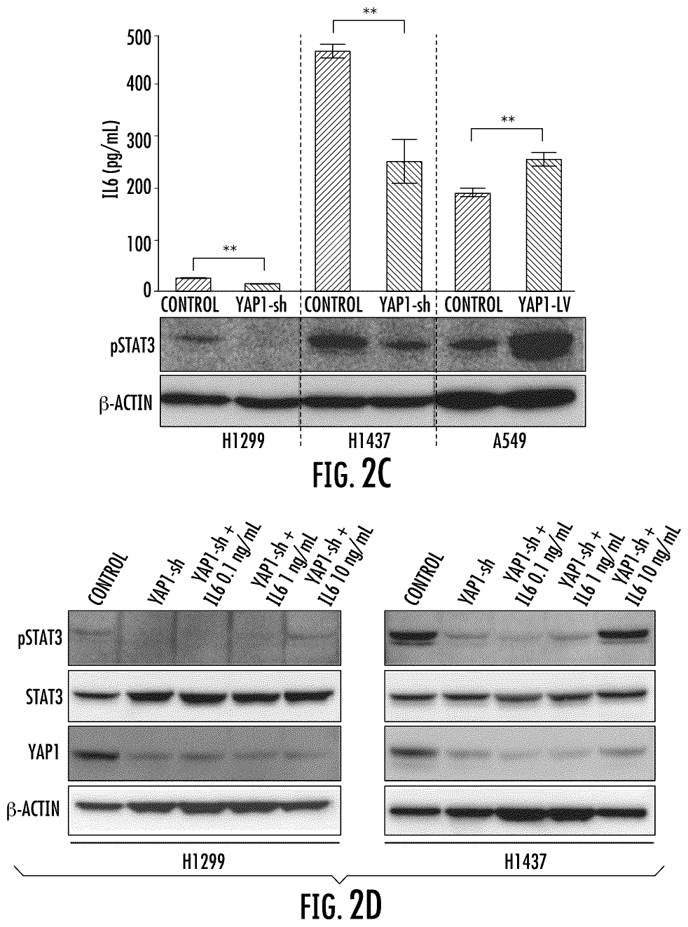

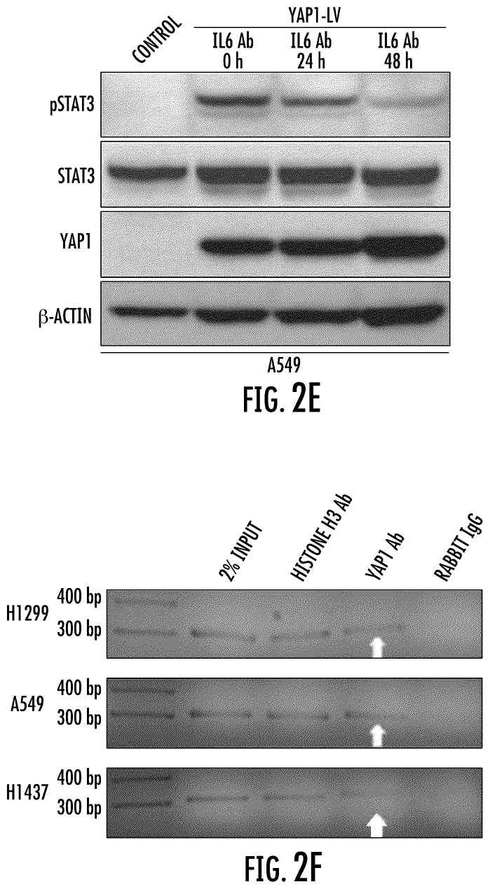

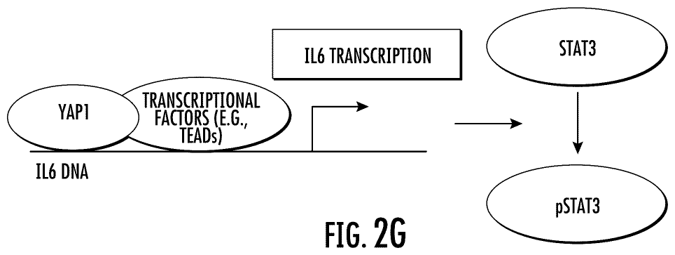

In further embodiments, YAP1 inhibitors include, but are not limited to, Narciclasine (Kawamoto et al., 1 BBA Advances 100008 (2021)); Dastinib (Omori et al. 6(12) Sci. Advances eaay3324 (2020)); Fluvasatin; Simvastatin; Rock inhibitor Y-27632; CA3 (WO2008140792; US20150157584), A413 (U.S. Pat. No. 2,870,146); A414 (US20090163545); A432 (Xu et al., 5 Sci. Rep. 10043 (2015)); and A433 (Song et al., 17(2) Mol. Cancer Therapeutics 443-54 (2018)) (See FIG. 1E and Supplemental FIGS. 1 and 2).

Other YAP1 inhibitors include, but are not limited to, Protoporphyrin IX, Zoledronic acid, Super-TDU, Auranofin, Metformin, Ivermectin and Milbemycin-D, Latrunculin A, Okadaic acid, Simvastatin, Staurosporine, Clomipramine, Heclin Dasatinib, Wortmannin, 4-((4-(3,4-Dichlorophenyl)-1,2,5-thiadiazol-3-yl)oxy)butane-1-ol, 4-[2-[4-(4-Hydroxyphenyl)butan-2-ylamino]ethyl]benzene-1,2-diol (Dobutamine), 4-[(1 R)-1-Hydroxy-2-(methylamino)ethyl]benzene-1,2-diol (Epinephrine), 5-(2-phenylpyrazolo[1,5-a]pyridin-3-yl)-2H-pyrazolo[3,4-c]pyridazin-3-amine, 4-{[(1 S)-1-Carboxy-3-methylbutyl]carbamoyl}-/V-[(1H-imidazol-4-yl)methyl]-3-(naphthalen-1-yl)-anilinium trifluoroacetate, 2-[4-(Trifluoromethyl)phenyl]-1,5,7,8-tetrahydrothiopyrano[4,3-d]pyrimidin-4-one, 4-[(1 R)-1-aminoethyl]-/V-pyridin-4-ylcyclohexane-1-carboxamide, Ac-KLRPVAMVRPVR-NH2 (SEQ ID NO:1), or Ac-GRKKRRQRRRPQKLRPVAMVRPVR-NH2 (SEQ ID NO:2) (39).

### III. STAT3 Inhibitors

A STAT3 inhibitor can include an a, b-unsaturated carboxamide containing compound such as WP1066 and WP1732. See WO2018232252 (WP1066, S3I-201, claims 21-24) and WO202006105 (WP1732). The family of a, b-unsaturated carboxamide-containing compounds contemplated for use in the present methods include those described in specifications and claims of U.S. Pat. Nos. 7,745,468; 8,119,827; 8,143,412; 8,450,337; 8,648,102; 8,779,151; 9,096,499; 8,809,377; and 9,868,736; U.S. application Ser. No. 16/185,669; U.S. Patent Appln. Publn. Nos. 2016/0237082; 2005/0277680; 2011/0021805; 2011/0053992; 2010/0152143; 2014/0329901; and 2012/0214850; and International (PCT) Appln. Publn. Nos. WO2010/005,807 and WO2015/187,427, each of which is incorporated by reference herein in its entirety.

A STAT3 inhibitor also includes 1-acetyl-5-hydroxyanthracene-9, 10-dione (CLT-005) (WO2015167567) (page 7, lines 10-29 through page 8, lines 1-10). A STAT3 inhibitor also comprises a Platinum [IV] compound including, but not limited to, CPA-1, CPA-3, CPA-7, platinum [IV]tetrachloride, IS3 295. See WO2006065894; see also compounds disclosed in US20050080131 (claims 1-16, Table 5 and paragraphs 0]-2])).

In particular embodiments, a STAT3 inhibitor comprises N-(1′,2-dihydroxy-1,2′-binaphthalen-4′-yl)-4-methoxybenzenesulfonamide, N-(3,1′-Dihydroxy-[1,2′]binaphthalenyl-4′-yl)-4-methoxy-benzenesulfonamide, N-(4,1′-Dihydroxy-[1,2′]binaphthalenyl-4′-yl)-4-methoxy-benzenesulfonamide, N-(5,1′-Dihydroxy-[1,2′]binaphthalenyl-4′-yl)-4-methoxy-benzenesulfonamide, N-(6,1′-Dihydroxy-[1,2′]binaphthalenyl-4′-yl)-4-methoxy-benzenesulfonamide, N-(7,1′-Dihydroxy-[1,2′]binaphthalenyl-4′-yl)-4-methoxy-benzenesulfonamide, N-(8,1′-Dihydroxy-[1,2′]binaphthalenyl-4′-yl)-4-methoxy-benzenesulfonamide, 4-Bromo-N-(1,6′-dihydroxy-[2,2′]binaphthalenyl-4-yl)-benzenesulfonamide, and 4-Bromo-N-[4-hydroxy-3-(1H-[1,2,4]triazol-3-ylsulfanyl)-naphthalen-1-yl]-benzenesulfonamide, or a pharmaceutically acceptable salt thereof. See WO20211113551, including the compounds described in ¶¶4]-55] and Tables 2-7.

### IV. Chemotherapy

The present invention also comprises administration of a chemotherapeutic agent. A “chemotherapeutic agent” or “chemotherapeutic compound” and their grammatical equivalents as used herein, can be a chemical compound useful in the treatment of cancer. The chemotherapeutic cancer agents that can be used in combination with a T cell include, but are not limited to, mitotic inhibitors (vinca alkaloids). These include vincristine, vinblastine, vindesine and Navelbine™ (vinorelbine, 5′-noranhydroblastine). In yet other cases, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide. The present disclosure further encompasses other chemotherapeutic cancer agents known as alkylating agents, which alkylate the genetic material in tumor cells. These include, without limitation, cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine. An additional category of chemotherapeutic cancer agents that may be used in the methods and compositions disclosed herein include antibiotics. Examples include without limitation doxorubicin, bleomycin, dactinomycin, daunorubicin, mithramycin, mitomycin, mytomycin C, and daunomycin. There are numerous liposomal formulations commercially available for these compounds. The present disclosure further encompasses other chemotherapeutic cancer agents including without limitation anti-tumor antibodies, dacarbazine, azacytidine, amsacrine, melphalan, ifosfamide and mitoxantrone.

A composition can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide.

Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the compositions described herein include, but are not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; avastin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idambicin hydrochloride; ifosfamide; ilmofosine; interleukin II (including recombinant interleukin II, or rIL2), interferon alfa-2a; interferon alfa-2b; interferon alfa-nl; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclambicin; acylfulvene; adecypenol; adozelesin; aldesleukin; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-I; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor I-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; Rll retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

### V. Immunotherapy

An immunotherapy can be administered to the patient in methods described herein. The term “immunotherapy” refers to a therapeutic treatment that involves administering to a patient an agent that modulates the immune system. For example, an immunotherapy can increase the expression and/or activity of a regulator of the immune system. In other instances, an immunotherapy can decrease the expression and/or activity of a regulator of the immune system. In some instances, an immunotherapy can recruit and/or enhance the activity of an immune cell. An example of an immunotherapy is a therapeutic treatment that involves administering at least one, e.g., two or more, immune checkpoint inhibitors. Exemplary immune checkpoint inhibitors useful in the presently-described methods are CTLA-4 inhibitors, PD-1 inhibitors or PD-L1 inhibitors, or combinations thereof.

The immunotherapy can be a cellular immunotherapy (e.g., adoptive T-cell therapy, dendritic cell therapy, natural killer cell therapy). For example, the cellular immunotherapy can be sipuleucel-T (APC8015; Provenge™; Plosker (2011) Drugs 71(1): 101-108). In some instances, the cellular immunotherapy includes cells that express a chimeric antigen receptor (CAR). In some instances, the cellular immunotherapy can be a CAR-T cell therapy, e.g., tisagenlecleucel (Kymriah™).

Immunotherapy be, e.g., an antibody therapy (e.g., a monoclonal antibody, a conjugated antibody). Exemplary antibody therapies are bevacizumab (Mvasti™, Avastin®), trastuzumab (Herceptin®), avelumab (Bavencio®), rituximab (Mab Thera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (ETnituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab or amatuximab.

An immunotherapy described herein can involve administering an antibody-drug conjugate to a patient. The antibody-drug conjugate can be, e.g., gemtuzumab ozogamicin (Mylotarg™), inotuzumab ozogamicin (Besponsa®), brentuximab vedotin (Adcetris®), ado-trastuzumab emtansine (TDM-1; Kadcyla®), mirvetuximab soravtansine (IMGN853) or anetumab ravtansine.

In some instances, the immunotherapy includes blinatumomab (AMG103; Blincyto®) or midostaurin (Rydapt).

An immunotherapy can include administering to the patient a toxin. For example, the immunotherapy can including administering denileukin diftitox (Ontak®).

In some instances, the immunotherapy can be a cytokine therapy. The cytokine therapy can be, e.g., an interleukin 2 (IL-2) therapy, an interferon alpha (IFN-α) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFN-α therapy is IntronA® (Roferon-A®). In some embodiments, the G-CSF therapy is filgrastim (Neupogen®).

In some instances, the immunotherapy is an immune checkpoint inhibitor. For example, the immunotherapy can include administering one or more immune checkpoint inhibitors. In some embodiments, the immune checkpoint inhibitor is a CTLA-4 inhibitor, a PD-1 inhibitor or a PD-L1 inhibitor. An exemplary CTLA-4 inhibitor would be, e.g., ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

In some instances, the immunotherapy is mRNA-based immunotherapy. For example, the mRNA-based immunotherapy can be CV9104 (see, e.g., Rausch et al. (2014) Human Vaccin Immunother 10(11): 3146-52; and Kubler et al. (2015) J. Immunother Cancer 3:26).

In some instances, the immunotherapy can involve bacillus Calmette-Guerin (BCG) therapy.

In some instances, the immunotherapy can be an oncolytic virus therapy. For example, the oncolytic virus therapy can involve administering talimogene alherparepvec (T-VEC; Imlygic®).

In some instances, the immunotherapy is a cancer vaccine, e.g., a human papillomavirus (HPV) vaccine. For example, an HPV vaccine can be Gardasil®, Gardasil9® or Cervarix®. In some instances, the cancer vaccine is a hepatitis B virus (HBV) vaccine. In some embodiments, the HBV vaccine is Engerix-B®, Recombivax HB® or GI-13020 (Tarmogen®). In some embodiments, the cancer vaccine is Twinrix® or Pediarix®. In some embodiments, the cancer vaccine is BiovaxID®, Oncophage®, GVAX, ADXS11-001, ALVAC-CEA, PROSTVAC®, Rindopepimut®, CimaVax-EGF, lapuleucel-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-l 10).

The immunotherapy can involve, e.g., administering a peptide vaccine. For example, the peptide vaccine can be nelipepimut-S (E75) (NeuVax™), IMA901, or SurVaxM (SVN53-67).

In some instances, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01. In some embodiments, the cancer vaccine is a DNA-based vaccine. In some embodiments, the DNA-based vaccine is a mammaglobin-A DNA vaccine (see, e.g., Kim et al. (2016) Oncolmmunology 5(2): e1069940).

### VI. Pharmaceutical Compositions and Administration

Accordingly, a pharmaceutical composition of the present invention may comprise a YAP1 inhibitor, a STAT3 inhibitor, a chemotherapeutic agent and/or immunotherapy. As used herein, the term “effective,” means adequate to accomplish a desired, expected, or intended result. More particularly, an “effective amount” or a “therapeutically effective amount” is used interchangeably and refers to an amount of a YAP1 inhibitor, a STAT3 inhibitor, a chemotherapeutic agent and/or immunotherapy, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from cancer. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which a YAP1 inhibitor, a STAT3 inhibitor, a chemotherapeutic agent and/or immunotherapy is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a YAP1 inhibitor, a STAT3 inhibitor, a chemotherapeutic agent and/or immunotherapy together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, subcutaneous injection is preferred.

In general, the pharmaceutical compositions comprising a YAP1 inhibitor, a STAT3 inhibitor, a chemotherapeutic agent and/or immunotherapy may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising a YAP1 inhibitor, a STAT3 inhibitor, a chemotherapeutic agent and/or immunotherapy, optionally in combination with yet another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effect is the therapeutic index and it may be expressed as the ratio LD50/ED50. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED50 with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC50 (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Concurrent Targeting of Potential Cancer Stem Cells Regulating Pathways Sensitizes Lung Adenocarcinoma to Standard Chemotherapy

Cancer stem cells (CSC) are highly resistant to conventional chemotherapeutic drugs. YAP1 and STAT3 are the two transcription factors that facilitate the therapeutic resistance and expansion of CSCs. The objective of this study was to understand the cross-talk between YAP1 and STAT3 activities and to determine the therapeutic efficacy of targeting dual CSC-regulating pathways (YAP1 and STAT3) combined with chemotherapy in lung adenocarcinoma. Here, we showed that YAP1 contributes to CSC regulation and enhances tumor formation while suppressing apoptosis. Mechanistically, YAP1 promotes phosphorylation of STAT3 by upregulating IL6. In lung adenocarcinoma clinical specimens, YAP1 expression correlated with that of IL6 (P<0.01). More importantly, YAP1 and phosphorylated STAT3 (pSTAT3) protein expressions were significantly correlated (P<0.0001) in primary lung adenocarcinoma as determined by IHC. Immunoblotting of 13 lung adenocarcinoma patient-derived xenografts (PDX) showed that all YAP1-expressing PDXs also exhibited pSTAT3. Additional investigations revealed that chemotherapy resistance and malignant stemness were influenced by upregulating NANOG, OCT4, and SOX2, and the expression of these targets significantly attenuated by genetically and pharmacologically hindering the activities of YAP1 and STAT3 in vivo and in vitro. Therapeutically, the dual inhibition of YAP1 and STAT3 elicits a long-lasting therapeutic response by limiting CSC expansion following chemotherapy in cell line xenograft and PDX models of lung adenocarcinoma. Collectively, these findings provide a conceptual framework to target the YAP1 and STAT3 pathways concurrently with systemic chemotherapy to improve the clinical management of lung adenocarcinoma, based on evidence that these two pathways expand CSC populations that mediate resistance to chemotherapy.

### Materials and Methods

Cell lines and gene modification. EGFR wild-type lung adenocarcinoma cell lines (A549, H1299, and H1437) were obtained from ATCC. Cells were stored at −80° C. and cultured in RPMI1640 (Mediatech) for H1299 and H1437, and F-12K (Mediatech) for A549, supplemented with 10% FBS (Atlanta Biologicals) in an atmosphere containing 5% CO2 at 37° C. All cell lines were determined to be Mycoplasma free.

Gene silencing of YAP1 was performed using YAP1 shRNA pGFPC-shLenti Vector (YAP1-sh) purchased from OnGene (#TL308332). For YAP1 and STAT3 overexpression, YAP1-inducible lentivirus (YAP1-LV) and STAT3-inducible lentivirus (STAT3-LV) were purchased from GenTarget (#LVP478 and #LVP383). To establish YAP1-knockdown and STAT3-overexpressing cells (YAP1-sh/STAT3-LV), YAP1-sh cells were transduced with STAT3-LV as described above. Genetic inhibition of STAT3 (STAT3-si) was conducted with STAT3 Silencer siRNA (Thermo Fisher Scientific; #AM16708). Procedures for these genetic modifications are detailed in the Supplementary Data. Expression levels of targeted molecules in these knockdown or overexpressing cells were validated by both quantitative real-time reversetranscription PCR (qRT-PCR) and Western blotting.

Cell proliferation assay and viability assay. Cell proliferation and viability were evaluated using Cell Counting Kit-8 (Dojindo Molecular Technologies). For cell proliferation assay, after cells (5×103/well) were seeded into 96-well plates with RPMI1640 containing 2% FBS, the optical density of each well was measured every 24 hours for 4 days. We added 10 mL of cell counting kit-8 solution to each well 2 hours before measuring the absorbance (17). For the cell viability assay, after cells were incubated for 48 hours (5×103/well), they were exposed to each respective therapeutic agent for 72 hours. Cell viability for each agent was defined as the ratio of absorbance values of the treated cells to control cells (i.e., pharmacologically untreated or genetically unmodified). Absorbance values (450-630 nm) were measured by a Spectra Max 250 96-well Plate Reader (Molecular Devices). Each sample was applied to six wells, and their average rate was calculated. The therapeutic agents used here (in vitro) were cisplatin (Sigma-Aldrich; #P4394), S3I-201 (Santa Cruz Biotechnology; #sc-204304; ref. 18), and verteporfin (MedKoo; #203120).

Recombinant IL6 stimulation and neutralization of IL6. For exogenous IL6 stimulation, 5×105 cells were seeded in 10-cm dishes. After 48 hours, human recombinant IL6 (0.1, 1, or 10 ng/mL) from PeproTech was added to the cells. They were harvested after 48-hour incubation. To neutralize the IL6, Human/Primate IL6 Antibody (R&D Systems) was administered. We cultured 5×105 cells in 10-cm dishes. After 48 hours, antibody (1 mg/mL) was added to the cells. They were then harvested after 24 or 48 hours. Protein expression of each sample was evaluated by Western blotting.

Chromatin immunoprecipitation assay. Chromatin immunoprecipitation (ChIP) assay was performed with Simple ChIP Enzymatic Chromatin IP Kit (Cell Signaling Technology). After nuclei preparation and chromatin digestion, ChIP was performed with YAP1 antibody (D8H1X; Cell Signaling Technology; #14074) diluted to 1:50. Extracted DNA lysate that had not been subjected to ChIP was defined as “input” and diluted to 2% for PCR. The primers specific for the IL6 gene promoter region were (forward) 5′-CTGCAAGTTCCCACAGTTCA-3′ (SEQ ID NO:3) and (reverse) 5′-CCCACCT TCTTCAAAATCCA-3′ (SEQ ID NO:4), which generated a 304-bp product (19).

Clinical samples, tissue microarray, and IHC. To assess correlations among mRNA levels, we collected pathologically confirmed lung adenocarcinoma samples from 59 patients who underwent resections at the Johns Hopkins Hospital (Baltimore, MD), the Johns Hopkins Bayview Medical Center (Baltimore, MD), and the Medical College of Wisconsin Froedtert Memorial Hospital (Milwaukee, WI; ref. 20). Formalin-fixed, paraffin-embedded tissue microarray (TMA) sections were constructed from blocks of resected specimens from 165 patients with lung adenocarcinoma who were treated at the Sacro Cuore Don Calabria Hospital (Negrar, Verona, Italy; ref. 21). None of the patients received any therapies before their resections. Antibodies for YAP1 (EP1674Y; Abcam; #ab52771; 1:300) and pSTAT3 (D3A7; Cell Signaling Technology; #9145; 1:25) were used for IHC, which were scored blindly by a pathologist (E. Gabrielson). Intensity scores (0, none; 1+, weak; 2+, moderate; and 3+, strong) of nuclei staining were evaluated by light microscopy; a score of 0 was considered negative, and scores of 1+, 2+, or 3+ were considered positive.

In vivo tumor formation assay and xenograft treatment. For the tumor formation assay in mice, A549 (5×106 cells), H1299 (1×106 cells), or H1437 (5×106 cells) was suspended in a mixture of 50 mL serum-free DMEM (Mediatech) and 50 mL Cultrex Stem Cell Qualified, Reduced Growth Factor Basement Membrane Matrix (Trevigen) and injected subcutaneously to both flanks of 4- to 5-week-old athymic (nu+/nu+) mice (3 mice, six tumors for each group) that were obtained from Envigo. Tumor volume was calculated as (volume) ¼ (larger diameter)×(smaller diameter)2×½.

For therapeutic experiments, 4- to 5-week-old NOD/SCID/IL2Ry−/− (NSG) mice were obtained from the Johns Hopkins Medical Institute's (Baltimore, MD) animal care facility. We injected H1299 cells (1×106) and H1437 cells (1×106), which were suspended in the mixture as described above, subcutaneously into both flanks. For the patient-derived xenograft (PDX) models, 13 PDX blocks (CTG0162, 0178, 0502, 0848, 1309, 1351, 1358, 1361, 1762, 1885, 2017, 2481, and 2708) were obtained from Champion Oncology. All these PDXs were established from primary lung adenocarcinoma tumors. The tumor tissues were cut into 4×2 mm pieces and implanted in the subcutaneous spaces of both flanks. When tumor volumes reached to 200+50 mm3, mice were randomly assigned to six experimental groups [control, chemo (cisplatin+gemcitabine), chemo+verteporfin, chemo+S3I-201, “all” (cisplatin+gemcitabine+verteporfin+S3I-201), and verteporfin+S3I-201] at 5 mice per group. Cisplatin (Sigma-Aldrich; #P4394), gemcitabine (Medkoo; #100410), S3I-201 (MedKoo; #202541), and verteporfin (Sigma-Aldrich; #1711461) were used to treat the mice. Cisplatin (2.5 mg/kg) and gemcitabine (120 mg/kg) were administrated via intraperitoneal injection once a week (22). S3I-201 (5 mg/kg) was administrated via intraperitoneal injection every day (23). Verteporfin (50 mg/kg) was administrated via intraperitoneal injection three times a week (6). Control mice were injected with same volume (100 ml) of 10% DMSO in 1% Tween80 (Sigma-Aldrich). Each tumor volume was measured every 3 days. Therapeutic efficacy was evaluated from percentage change of the tumor volume compared with the tumor size before treatment.

In the PDX models, 1 mouse in each treatment arm was sacrificed separately in the middle of the treatment, at 18 days, to study pharmacodynamics. Their subcutaneous tumors were resected for Western blotting. These mice were not included in the tumor growth analysis.

All mice experiments were approved by the Johns Hopkins University Animal Care and Use Committee (Baltimore, MD, #MO17M142 and #MO18M20), and mice were maintained in accordance with the American Association of Laboratory Animal Care guidelines.

Statistical analysis. Differences between two groups were evaluated using Mann-Whitney test. When multiple groups were compared, we used the Kruskal-Wallis test with the post hoc test (Steel-Dwass test). Spearman rank correlation coefficient was employed to investigate correlation of continuous variables between two groups. Categorical variables were analyzed with ×² test. Estimated variations are indicated in each graph as SEM. All statistical analyses were performed on JMP 12 Software (SAS Institute). P<0.05 was considered significant.

Gene silencing and overexpression. Gene silencing of YAP1 was performed using YAP1 shRNA pGFP-C-shLenti Vector (YAP1-sh) purchased from Origene (Rockville, USA; #TL308332). shRNA with Lenti-vpak Lentiviral Packaging Kit (Origene) was transfected into 293T cells according to the manufacture's protocol to form lentiviral particles. Cells were seeded in 6-well plates for transduction. After 24 hours, lentiviral particles were added to the cells with 8 μg/ml polybrene (Millipore Sigma, Burlington, USA) and incubated overnight. Non-specific shRNA vector (Origene; #TR30021) was used as control. After transduction, cells were cultured in the medium with the optimal concentration of puromycin (Thermo Fisher Scientific, Waltham, USA). For YAP1 and STAT3 overexpression, YAP1-inducible lentivirus (YAP1-LV) and STAT3-inducible lentivirus (STAT3-LV) were purchased from GenTarget (San Diego, USA; #LVP478 and #LVP383). Cells were seeded in 24-well plates for transduction. After 24 hours, the lentivirus was added to the cells with 8 μg/ml polybrene. CMV control lentivirus (GenTarget; #CMV-Null-RB) was used as a control. The optimal concentration of blasticidin (InvivoGen, San Diego, USA) was added to the medium for the antibiotic selection. To establish YAP1-knockdown/STAT3-overexpressing cells (YAP1-sh/STAT3-LV), YAP1-sh cells were transduced with STAT3-inducible lentivirus as described above. Genetic inhibition of STAT3 (STAT3-si) was conducted with STAT3 Silencer siRNA (Thermo Fisher Scientific; #AM16708) at the final concentration of 80 nM. For the transfection, siRNA was added combined with Lipofectamine RNAiMAX (Thermo Fisher Scientific). Silencer Select Negative Control No. 1 siRNA (Thermo Fisher Scientific) served as a control nontargeting siRNA. The expression levels of targeted molecules in these knockdown or overexpressing cells were validated by both quantitative real-time reverse-transcription PCR (qRT-PCR) and western blotting.

qRT-PCR. The mRNA expression level of each gene was determined by qRT-PCR. Total RNA was extracted from respective cells using RNeasy Mini Kit (Qiagen, Valencia, USA). cDNA was synthesized from total RNA by M-MLV Reverse Transcriptase (Thermo Fisher Scientific) and Primer “random” (Sigma-Aldrich, St Louis, USA). qRT-PCR was performed using Fast SYBR Green Master Mix (Thermo Fisher Scientific) on a 7900HT Fast Real-Time PCR System (Thermo Fisher Scientific). Primer sequences, product size and annealing temperature for each gene are listed in Table 1. We quantified the β-actin mRNA level to normalize the expression levels of the targeted genes. Each sample was tested in triplicate and run three times. Each graph shown in our figures indicates the average mRNA expression level of targeted genes compared with the β-actin expression level, with standard error of mean.

Western blotting. Cells were dissolved in RIPA buffer (Thermo Fisher Scientific) supplemented with proteinase inhibitor and phosphatase inhibitor cocktails (Roche, Mannheim, Germany). After total protein lysate was electrophoretically transferred onto PVDF membrane, the membrane was blocked with 5% skim milk in 0.05% TBS-Tween buffer and incubated at 4° C. overnight with a primary antibody specific to each protein. The membrane was then probed with an appropriate secondary antibody (Cell Signaling Technology, Danvers, USA). β-actin served as an endogenous control. The primary antibodies and dilution ratios were β-actin (Sigma-Aldrich; #A2228; 1:5000), NANOG (D73G4; Cell Signaling Technology; #4903; 1:1000), OCT4 (D705Z; Cell Signaling Technology; #75463; 1:500), SOX2 (EPR3131; Abcam, Cambridge, UK; #ab92494; 1:500), STAT3 (124H6; Cell Signaling Technology; #9139; 1:2000), pSTAT3 (D3A7; Cell Signaling Technology; #9145; 1:1000) and YAP1 (EP1674Y; Abcam, #ab52771; 1:2000).

Apoptosis assay. Cells were incubated for 72 hours with CDDP (2 μM or 5 μM) or normal saline. Apoptosis assays were conducted using PE Annexin V Apoptosis Detection Kit with 7-AAD (BioLegend, San Diego, USA) according to manufacturer's protocol. Treated cells were analyzed by BD FACSCalibur flow cytometer (BD Biosciences, San Jose, USA).

Immunoprecipitation. To evaluate protein-protein interaction, we used SureBeads Protein G Magnetic Beads (Bio-Rad, Hercules, USA) for magnetization. After YAP1 antibody was diluted to 1:70 (EP1674Y; Abcam; #ab52771) and magnetized, 200 μg of each protein lysate was added. Normal Rabbit IgG (Cell Signaling Technology; #2729) was served as a negative control. After the lysate was eluted from magnetic beads, western blotting was performed. Non-magnetized protein (20 μg) of each sample was used as “Input”. The primary antibodies and dilution ratios used in the western blotting were β-actin (Sigma-Aldrich; #A2228; 1:5000), STAT3 (124H6; Cell Signaling Technology; #9139; 1:2000) and YAP1 (1A12; Cell Signaling Technology; #12395; 1:2000).

Enzyme-linked immunosorbent assay (ELISA). We performed ELISA to quantify IL-6 with Human IL-6 uncoated ELISA kit (Thermo Fisher Scientific), according to the manufacturer's protocol. We seeded 1×106 cells in 10-cm dishes and incubated them for 48 hours, then added 100 μl of each cell line medium to each well. Absorbance value (450-570 nm) was measured by a Spectra Max 250 96-well plate reader (Molecular Devices). Each sample was applied to eight wells, and their average concentration ratios were averaged.

Sphere formation assay. For sphere formation assay, cells (2×104/well) were cultured in DMEM/Ham's F12 50/50 Mix (Mediatech) supplemented with B-27 (Thermo Fisher Scientific), 20 ng/ml FGF-basic (Peprotech) and 20 ng/ml EGF (Peprotech). Cells were cultured in ultra-low attachment 6-well plates (Corning, Lowell, USA) for two weeks. New medium was added every three days. Sphere formation was evaluated with an inverted phase-contrast microscope; each single sphere with a diameter>100 μm was counted with NIS-Elements Microscope Imaging Software (Nikon, Tokyo, Japan). Each sample was applied to six wells, and their average numbers of spheres formed were calculated. These spheroid cells were harvested, and RNA was extracted to evaluate mRNA expression levels of CSC markers.

Isobologram analysis. We used isobologram analysis to evaluate the synergistic effect of verteporfin and S3I-201. First, the half-maximal inhibitory concentration (IC50) of each drug was determined by cell viability assays. The calculated ratios were indicated on the x and y axes of a two-coordinate plot as [(IC50 of verteporfin), 0] and [0, (IC50 of S3I-20I)]. The line connecting these two points was set as the standardized line. Cells were then treated with combinations of verteporfin and S3I-201 in various concentrations; concentrations that inhibit 50% of the cells were plotted in the same graph. Plots that were located below or above the standardized line were considered to indicate synergistic or antagonistic effects, respectively.

### Results

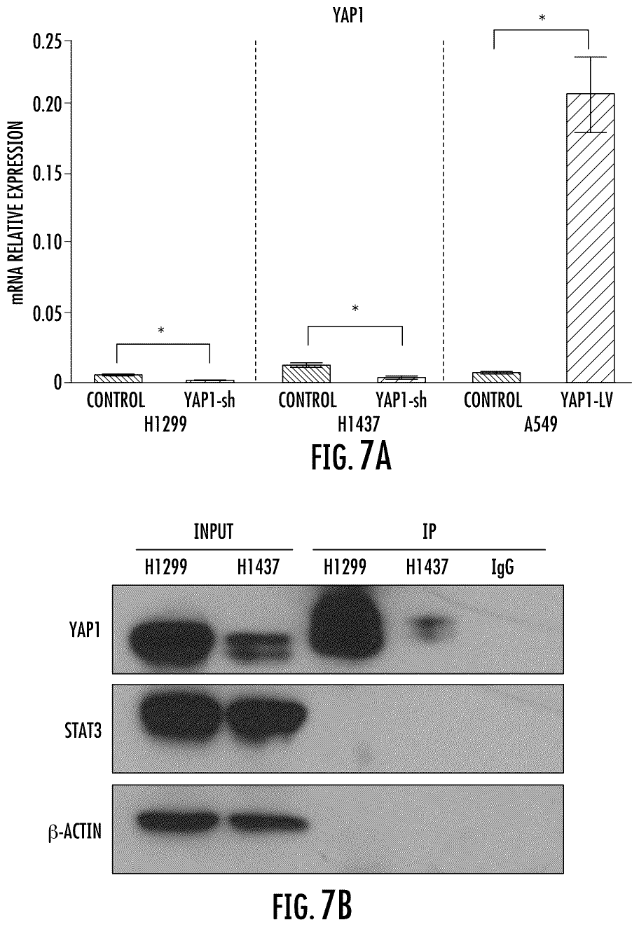

YAP1 promotes malignant phenotype in lung adenocarcinoma. As EGFR-targeting therapy is available for EGFR-mutant lung adenocarcinoma, we purposefully selected EGFR wild-type cell lines to identify new targets in this study. YAP1-knockdown lung adenocarcinoma cells (H1299 YAP1-sh and H1437 YAP1-sh) showed reduced proliferation, whereas YAP1-overexpressing cells (A549 YAP1-LV) showed increased proliferation rates (FIGS. 1A and B; FIG. 7A). When these cells were treated with cisplatin, both YAP1-sh cells showed increased sensitivities, whereas YAP1-LV cells were relatively resistant (FIG. 1C). YAP1-sh cells also showed higher fractions of apoptotic cells after cisplatin treatment, whereas YAP1-LV cells showed lower fractions (FIG. 1D). YAP1-sh cell lines showed suppressed tumor growth in athymic nude mice, whereas tumors formed by YAP1-LV cells grew more rapidly (FIG. 1E). Collectively, these results indicate that YAP1 promotes cell proliferation, drug resistance, and tumor growth, and suppresses apoptosis in lung adenocarcinoma.

YAP1 induces STAT3 phosphorylation through IL6 stimulation. As the YAP1 and the JAK/STAT3 pathways are reportedly linked (24), we performed immunoblotting analysis of YAP1 and STAT3 in YAP1-sh and YAP1-LV cells to explore any possible correlation. We observed a positive correlation between YAP1 and pSTAT3 expressions (FIG. 2A). As coimmunoprecipitation showed no evidence of direct binding between YAP1 and STAT3 (Supplementary FIG. SIB), we speculated that specific cytokines or growth factors stimulated by YAP1 may mediate STAT3 phosphorylation. Among mRNA expression levels of several analyzed molecules (EGF, IFNg, IL2, IL4, IL6, IL10, and TGFb) known to mediate STAT3 phosphorylation (25), only IL6 expression was positively associated with YAP1 expression (FIG. 2B; Supplementary FIG. Si C). In addition, IL6 protein levels in the cell culture media were significantly reduced in YAP1-sh cells, but were higher for YAP1-LV cells (FIG. 2C). Furthermore, exogenous IL6 increased pSTAT3 expressions in a dose-dependent manner without affecting total STAT3 protein levels in YAP1-sh cells (FIG. 2D). In contrast, IL6 neutralization antibody suppressed pSTAT3 in a time-dependent manner without affecting total STAT3 level in YAP1-LV cells (FIG. 2E). These results indicate that IL6 stimulated by YAP1 induces STAT3 phosphorylation. In addition, ChIP assay suggested that YAP1 protein directly binds to the IL6 gene promoter region (FIG. 2F), further confirming that YAP1 induces STAT3 activation via direct upregulation of IL6 (FIG. 2G).

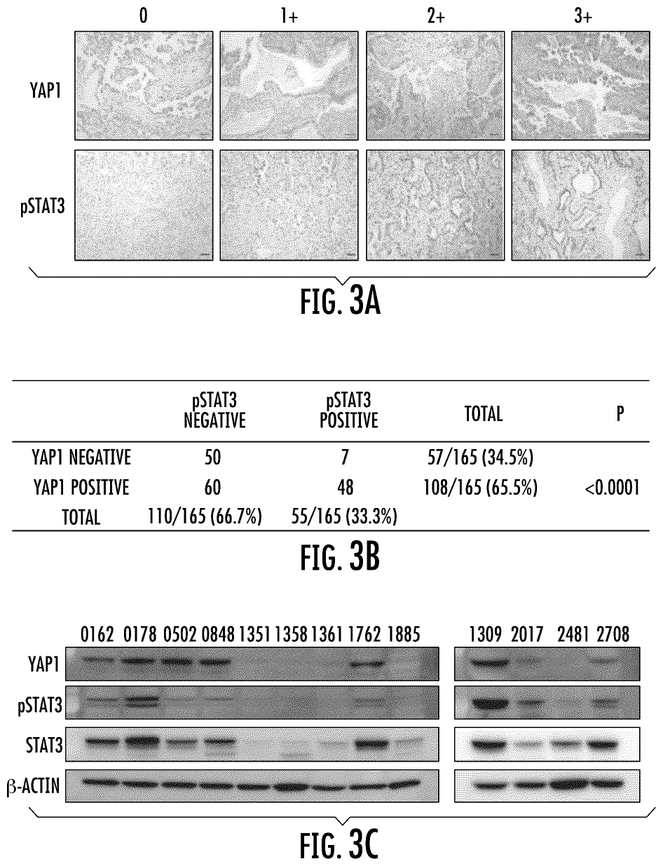

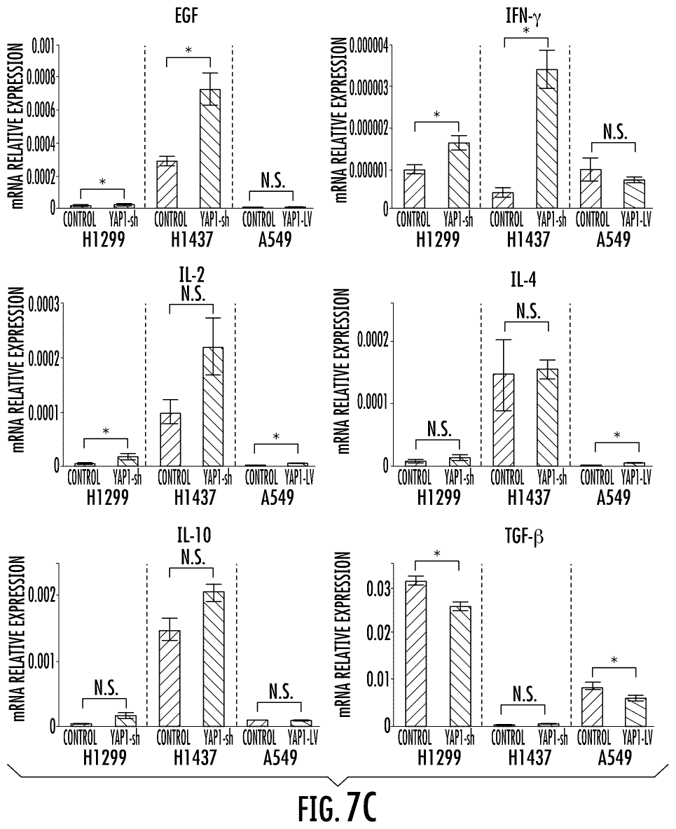

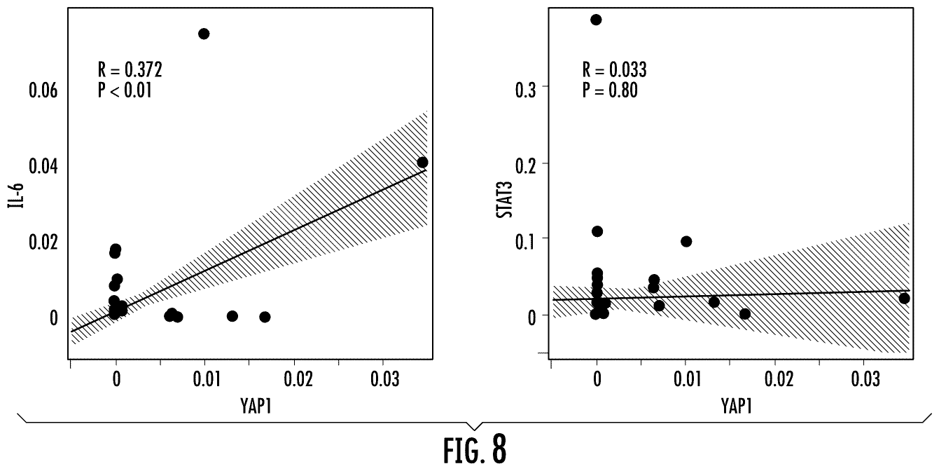

Correlations among YAP1, IL6, and pSTAT3 in human lung adenocarcinoma tumors. We found a positive correlation between YAP1 and IL6 mRNA expressions in 59 human lung adenocarcinoma samples (R=0.372; P<0.01), although there was no correlation between YAP1 and STAT3 mRNA expression levels (R=0.033; P<0.80; FIG. 8). At the protein level, nuclear staining of YAP1 and pSTAT3 was assessed by IHC in the TMA that contained 165 lung adenocarcinoma specimens. Representative staining for YAP1 and pSTAT3 is shown in FIG. 3A. YAP1 expression was negative in 34.5% cases (57/165) and positive in 65.5% cases (108/165; 1+, 28 cases; 2+, 35 cases; and 3+, 45 cases). pSTAT3 expression was negative in 66.7% cases (110/165) and positive in 33.3% cases (55/165; 1+, 21 cases; 2+, 30 cases; and 3+, four cases). Whereas 44.4% (48/108) of YAP1-positive cases expressed pSTAT3, 12.3% (7/57) of YAP1-negative cases expressed pSTAT3, indicating a significant correlation between YAP1 and pSTAT3 expressions (P<0.0001; FIG. 3B). In addition, immunoblotting of 13 lung adenocarcinoma PDXs showed that all YAP1-expressing PDXs also exhibited pSTAT3 (FIG. 3C). Taken together, these findings support our hypothesis and indicate a direct association between YAP1 and pSTAT3 signaling in lung adenocarcinoma tissues.

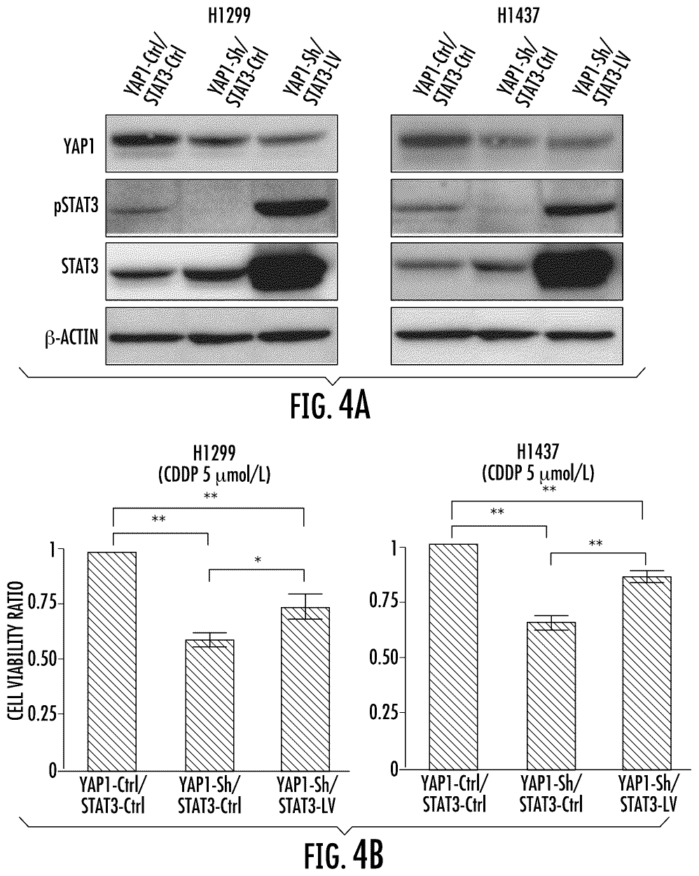

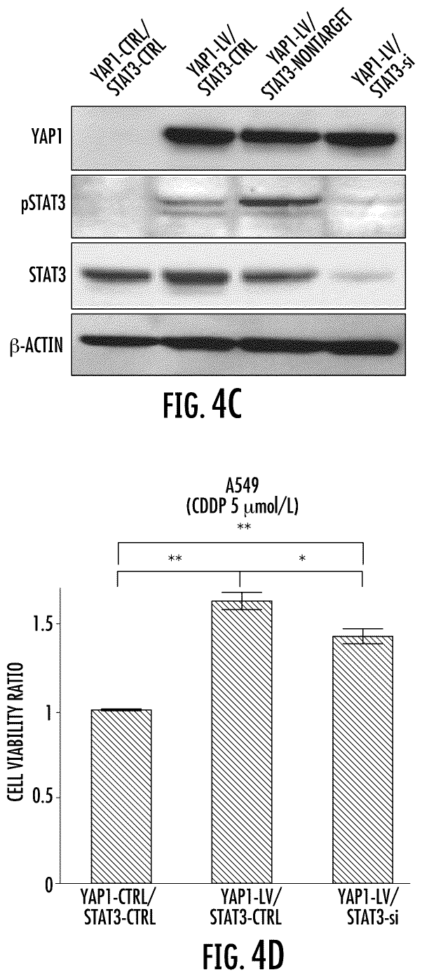

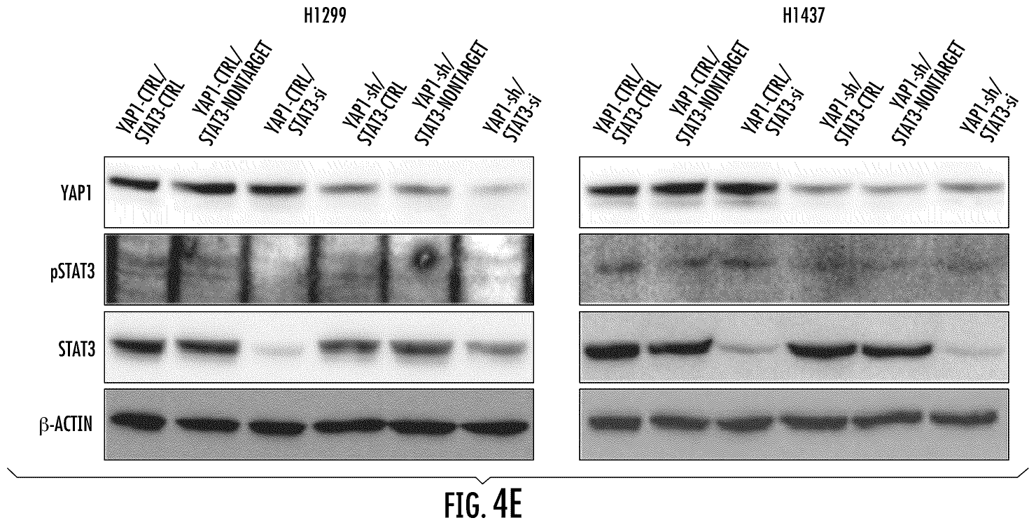

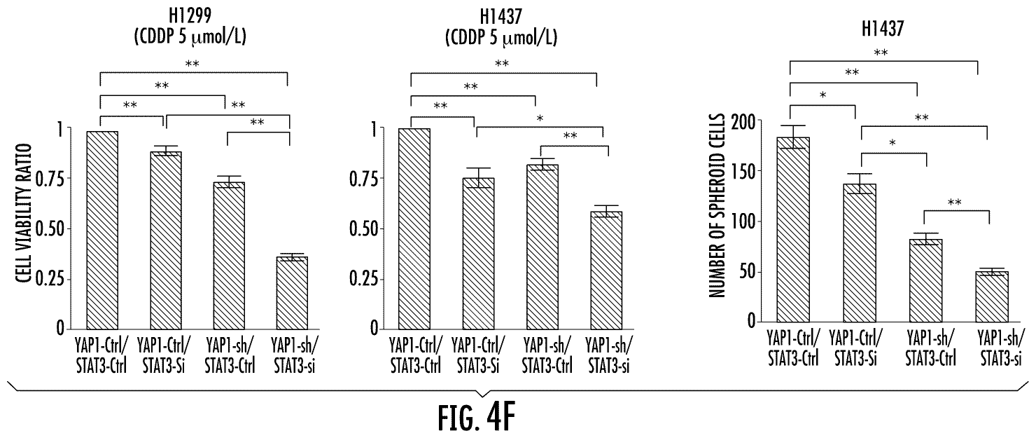

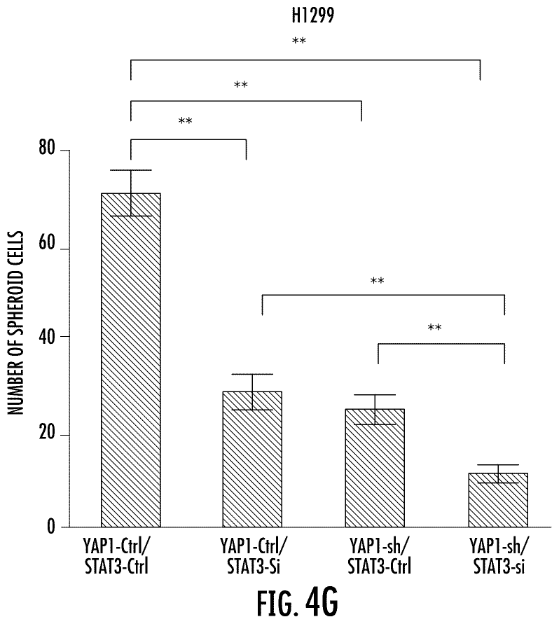

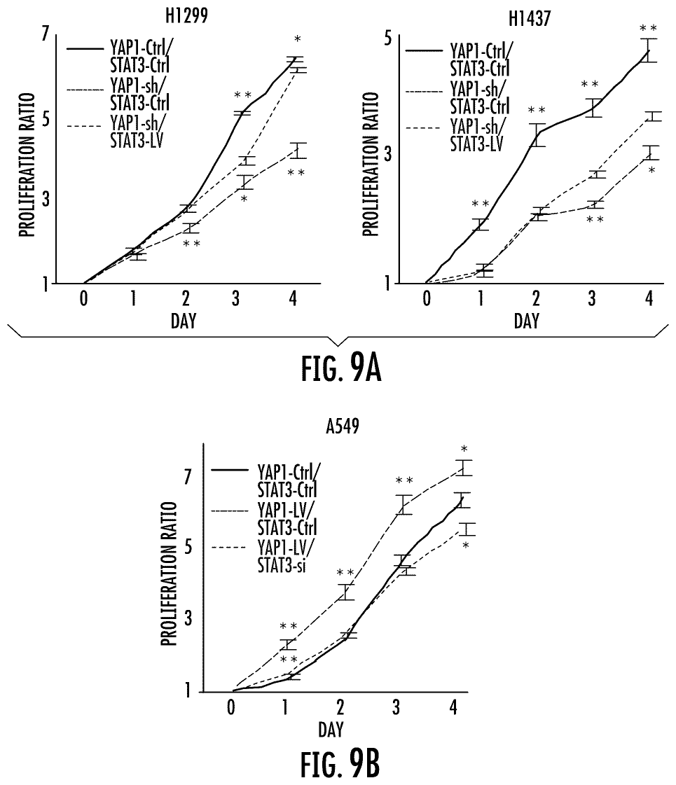

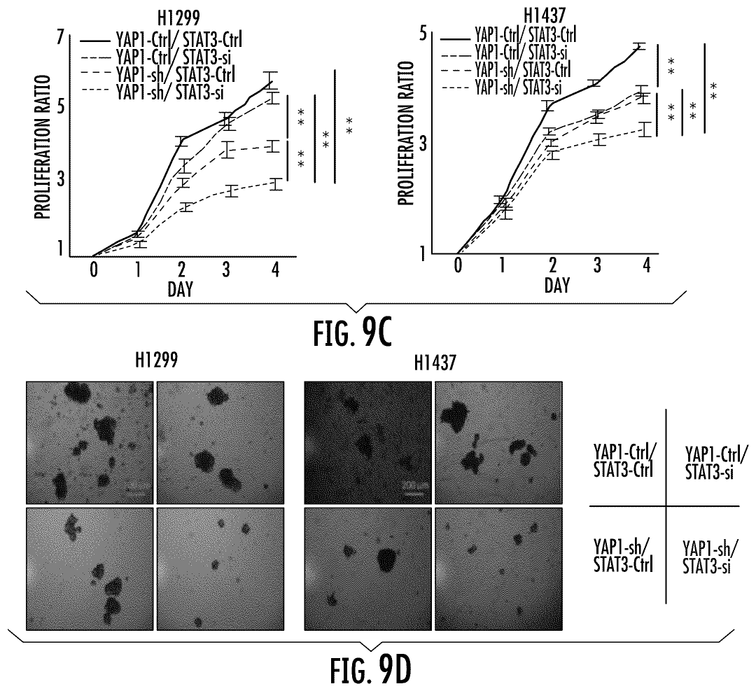

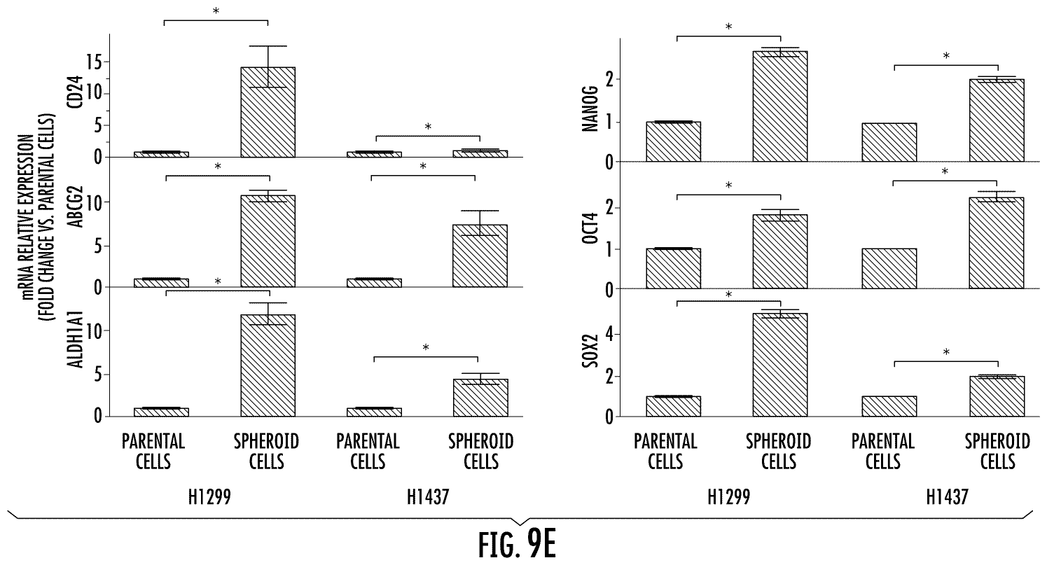

Tumor suppressive effects of dual genetic inhibition of YAP1 and STAT3. To evaluate the functional roles of the YAP1-IL6-pSTAT3 axis, STAT3 was genetically induced in YAP1-sh cells (FIG. 4A). Forced expression of STAT3 restored cell proliferation and cisplatin resistance that had been attenuated by YAP1 knockdown (FIG. 4B; FIG. 9A), whereas STAT3 knockdown in A549 YAP1-LV cells showed the opposite effects (FIGS. 4C and D; FIG. 9B), which implies a role for the YAP1-IL6-STAT3 axis in promoting malignant phenotypes. Interestingly, YAP1-sh/STAT3-LV cells showed higher proliferation and drug resistance than YAP1-sh/STAT3-Ctrl cells, but were still less aggressive than YAP1-Ctrl/STAT3-Ctrl cells (FIG. 4B; FIG. 9A). Furthermore, YAP1-LV/STAT3-si cells showed higher proliferation and drug resistance than YAP1-Ctrl/STAT3-Ctrl cells (FIG. 4D; FIG. 9B). These findings suggest that the oncogenic roles of YAP1 are not entirely STAT3 dependent, and YAP1 may drive tumorigenesis independently of the YAP1-STAT3 axis.

This evidence that, YAP1 has STAT3-independent tumor progressive roles led us to hypothesize that dual inhibition of YAP1 and STAT3 could synergistically suppress tumor growth. To test this hypothesis, we subsequently evaluated the effect of dual genetic blockade of YAP1 and STAT3 in lung adenocarcinoma cell lines (FIG. 4E) and found that dual inhibition of YAP1 and STAT3 resulted in greater suppression of cell proliferation and greater sensitivity to cisplatin than for inhibition of either pathway alone (FIG. 4F; FIG. 9C). Notably, whereas pSTAT3 expression levels upon dual inhibition were comparable with those of the YAP1-Ctrl/STAT3-si cells (FIG. 4E), the tumorigenic properties of dual pathway-inhibited cells were substantially reduced compared with single molecule inhibition (FIG. 4F; FIG. 9C). These findings suggest that the combinational blockade of YAP1 and STAT3 may effectively constrain cancer cell proliferation by inhibiting both the YAP1-IL6-STAT3 signaling axis, as well as other independent oncogenic pathways regulated by YAP1 and STAT3.

As both YAP1 and STAT3 reportedly promote cancer stemness-9 like features (3, 16), we hypothesized that inhibition of both molecules would synergistically inhibit sphere formation, a characteristic feature of CSCs. In support of this hypothesis, YAP1-sh/STAT3-si cells formed fewer and smaller spheroids than single target-inhibited cells or controls (FIG. 4G; FIG. 9D). The stemness-like features of these spheroids were verified at molecular level by confirming higher expression levels of known CSC markers in NSCLC that included ABCG2, ALDH1A1, CD24, NANOG, OCT4, and SOX2 (FIG. 9E). Collectively, these results suggest that dual genetic inhibition of YAP1 and STAT3 suppresses cancer cell growth and enhances chemotherapeutic sensitivity by attenuating cancer stemness-like features more efficiently than by inhibiting YAP1 or STAT3 alone.

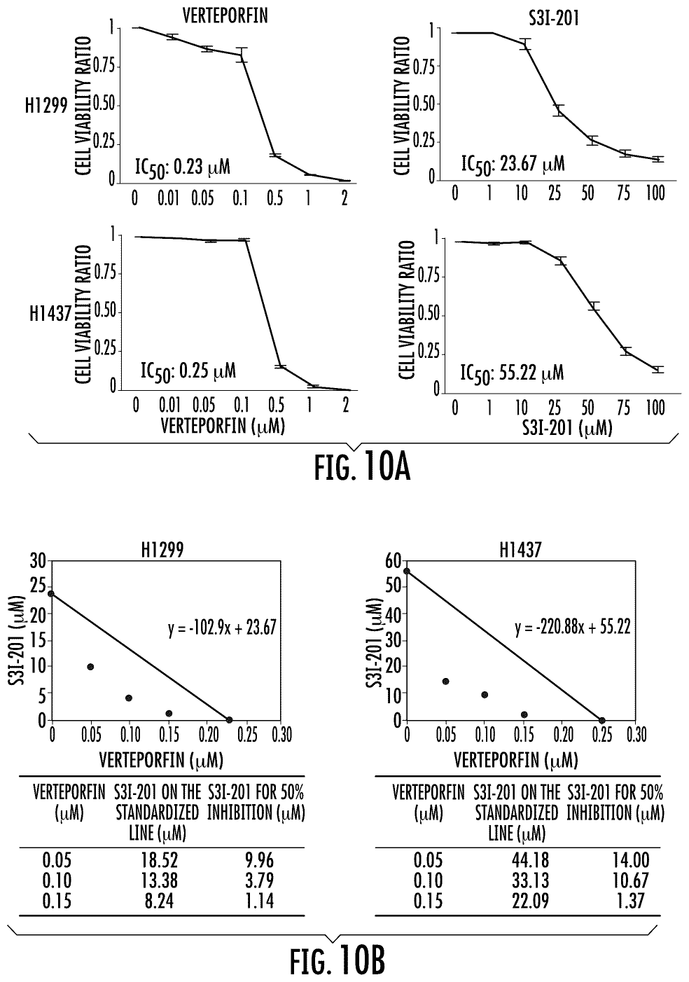

Effects of pharmacologic inhibition of YAP1 and STAT3 on cell growth. Verteporfin (YAP1 inhibitor) and S3I-201 (STAT3 inhibitor) were evaluated for synergistic effects by isobologram analysis. After IC50 of each drug was determined for H1299 and H1437 cells (FIG. 10A), IC50s for S3I-201 were determined when combined with 0.05, 0.10, or 0.15 mmol/L of verteporfin. As all IC50 values were plotted below the standardized line, verteporfin and S3I-201 were considered to have synergistic cytotoxic effects (FIG. 10B).

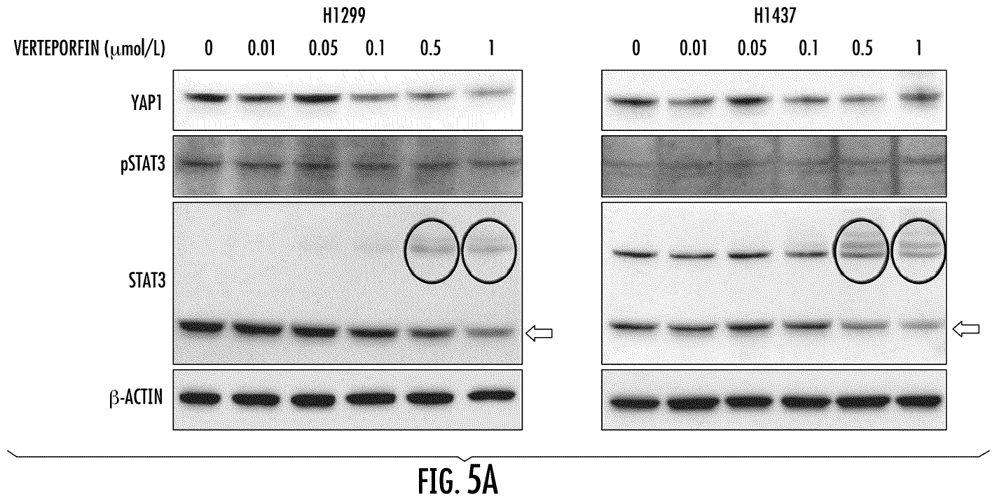

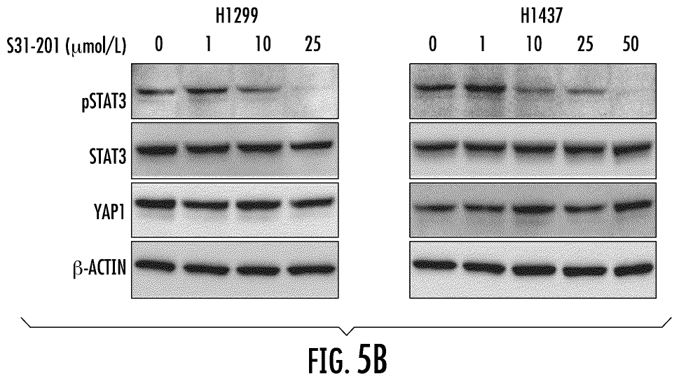

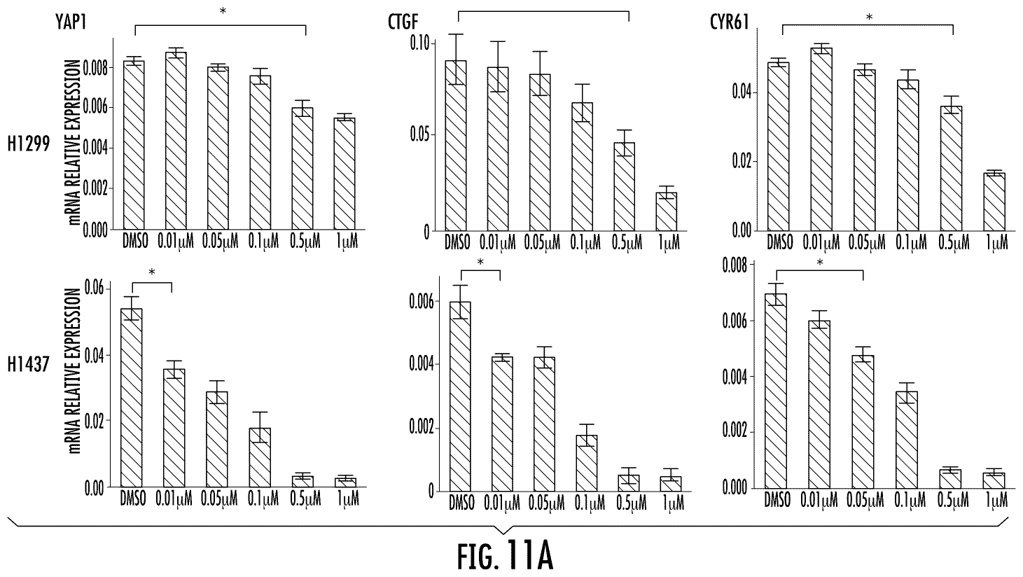

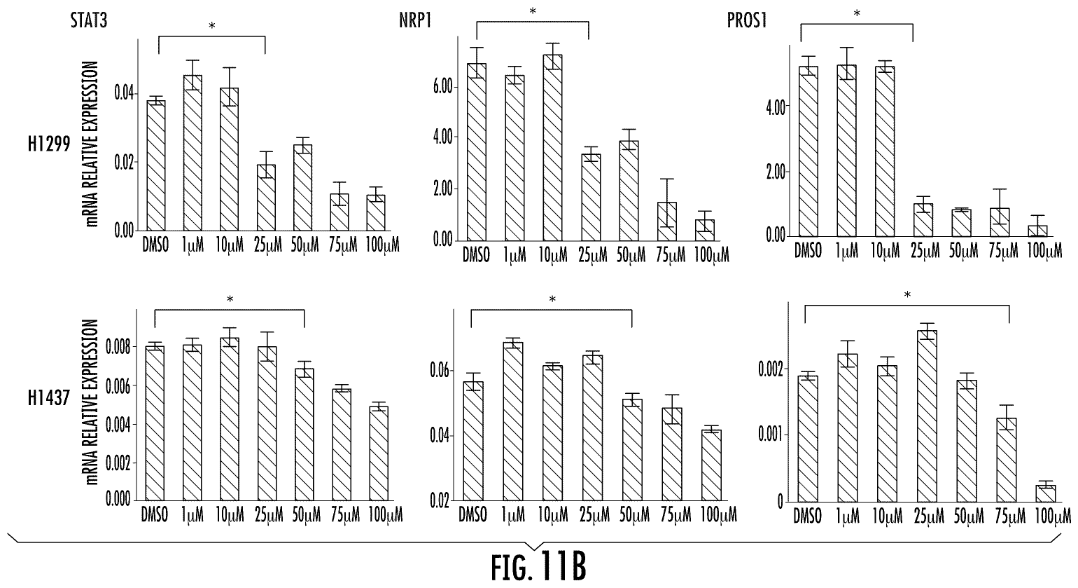

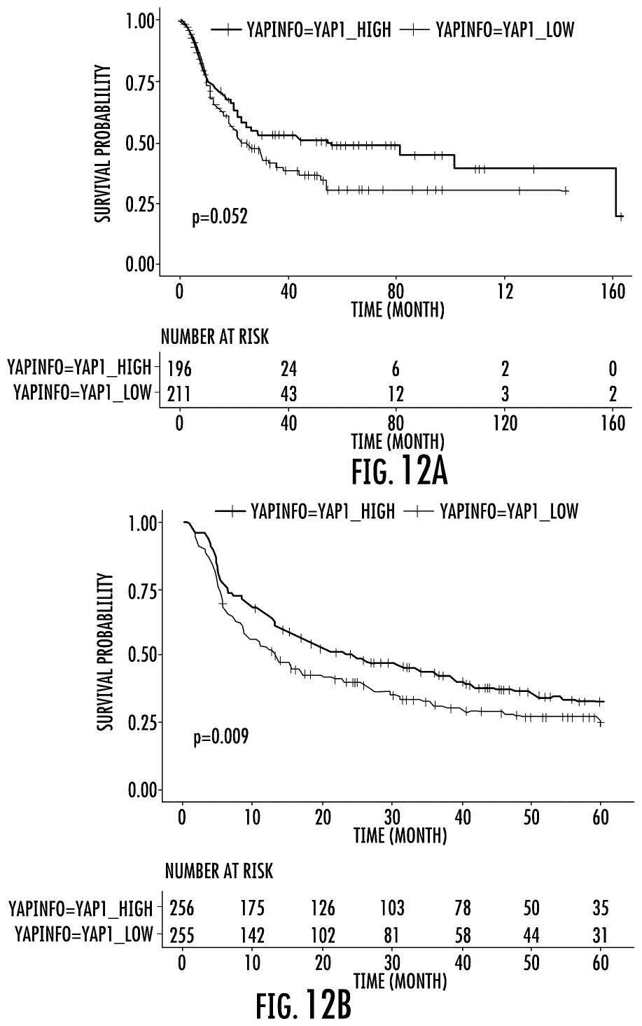

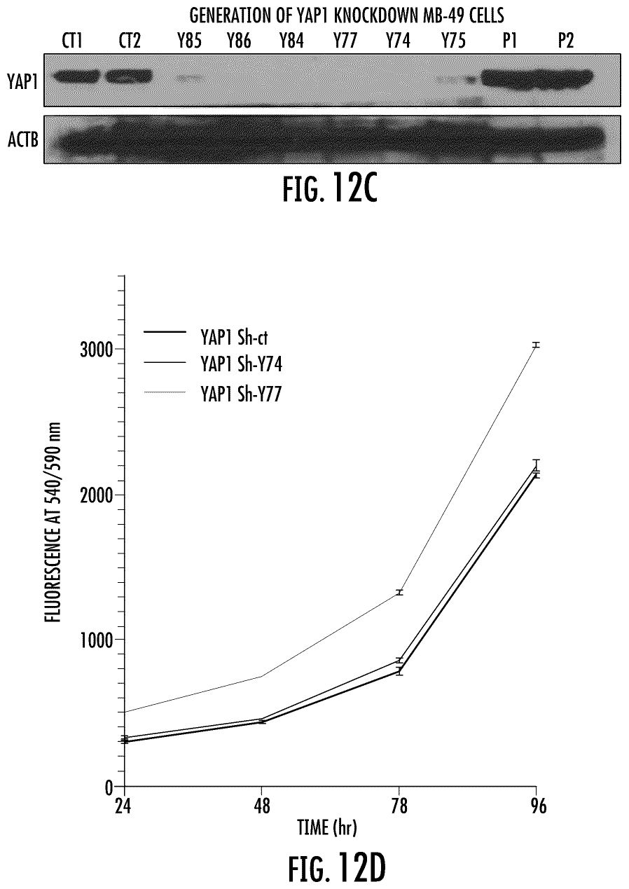

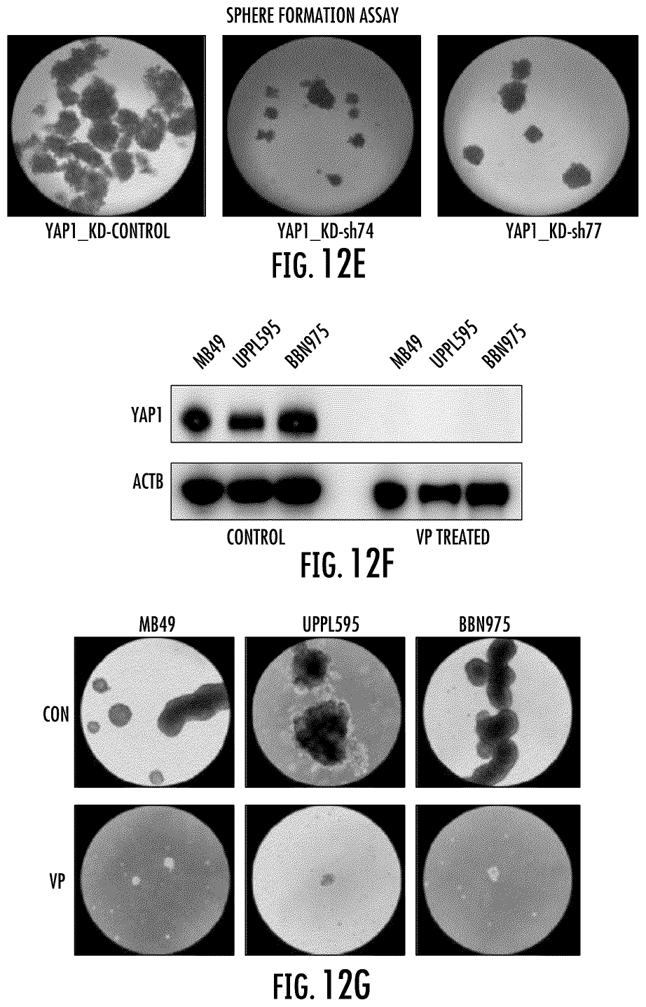

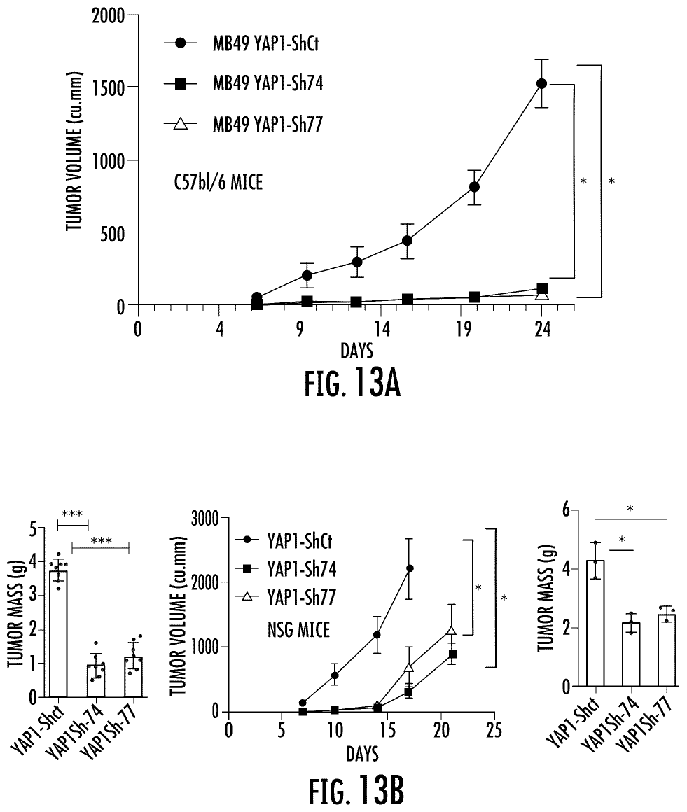

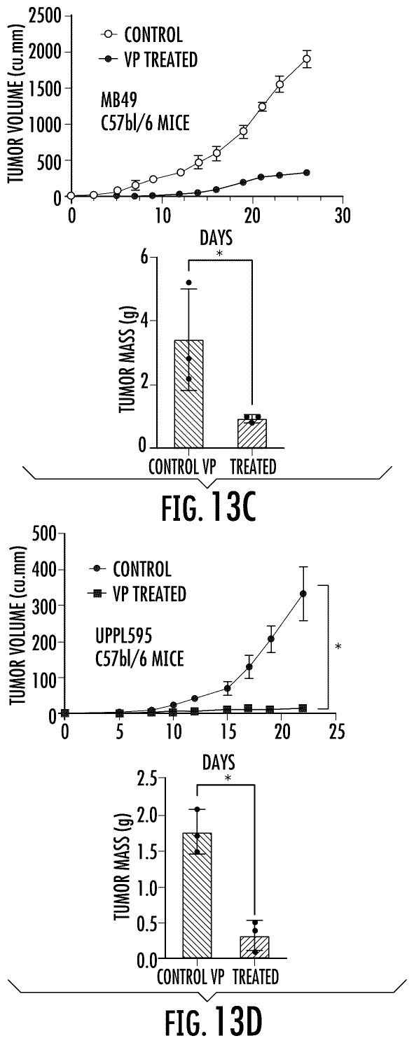

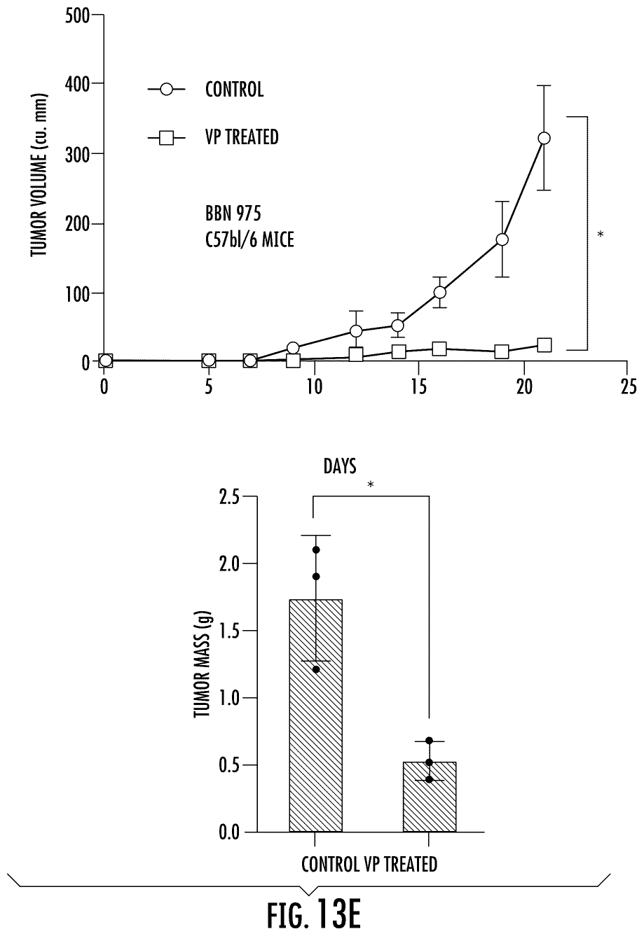

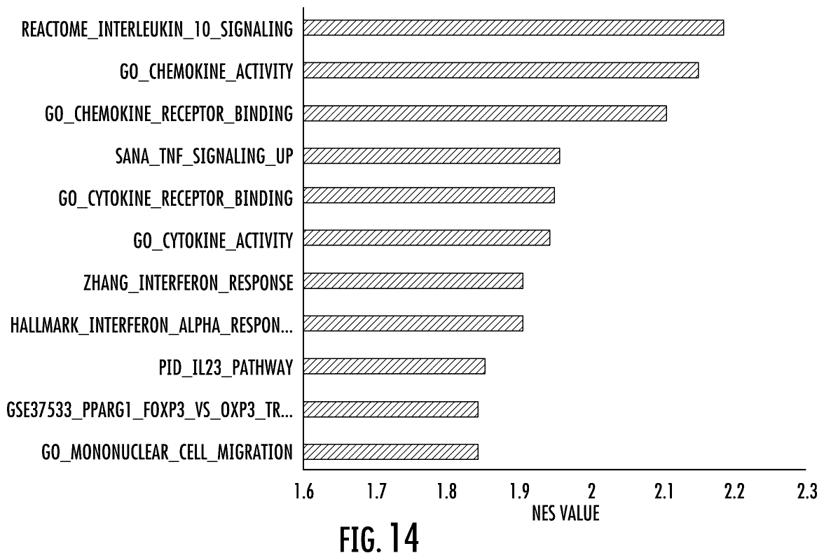

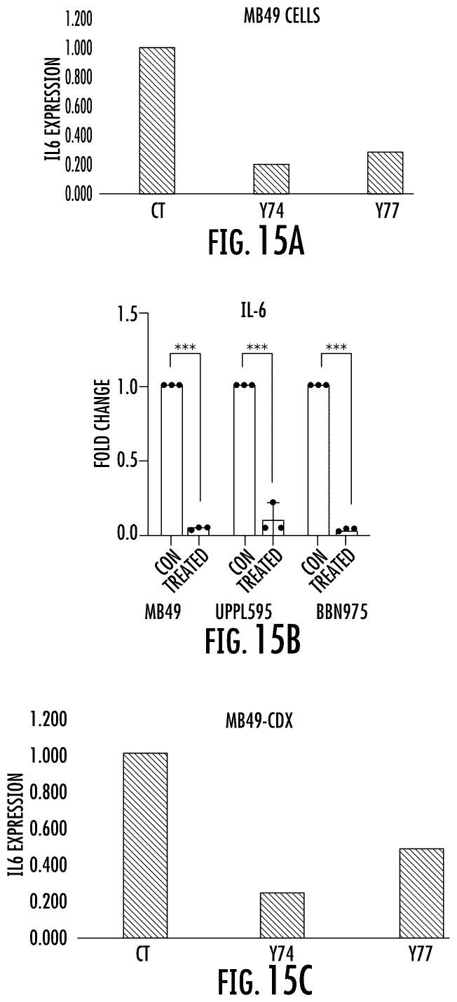

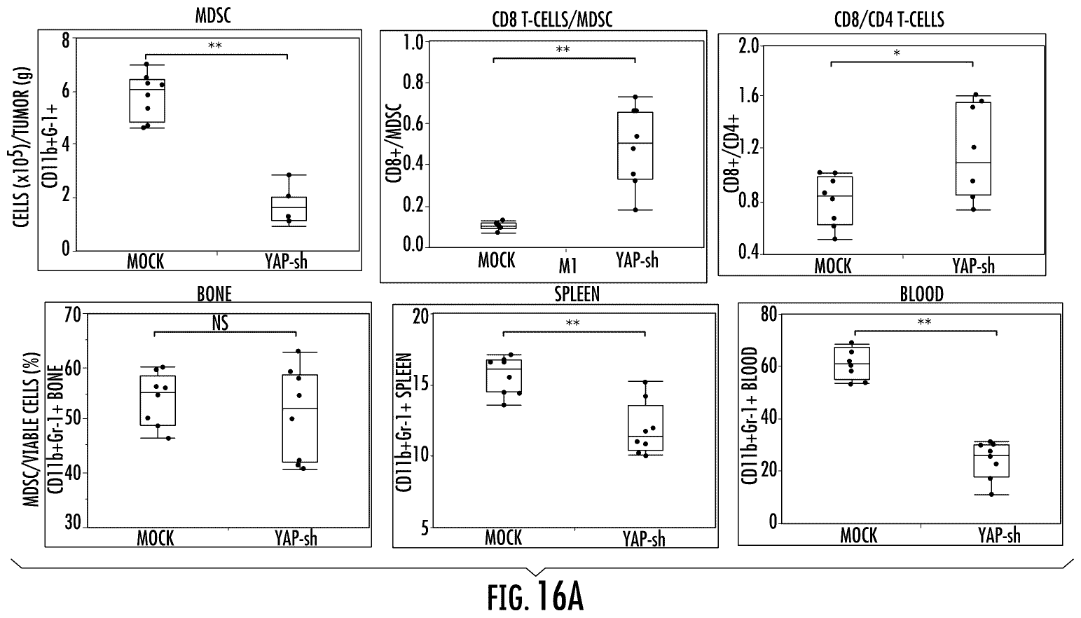

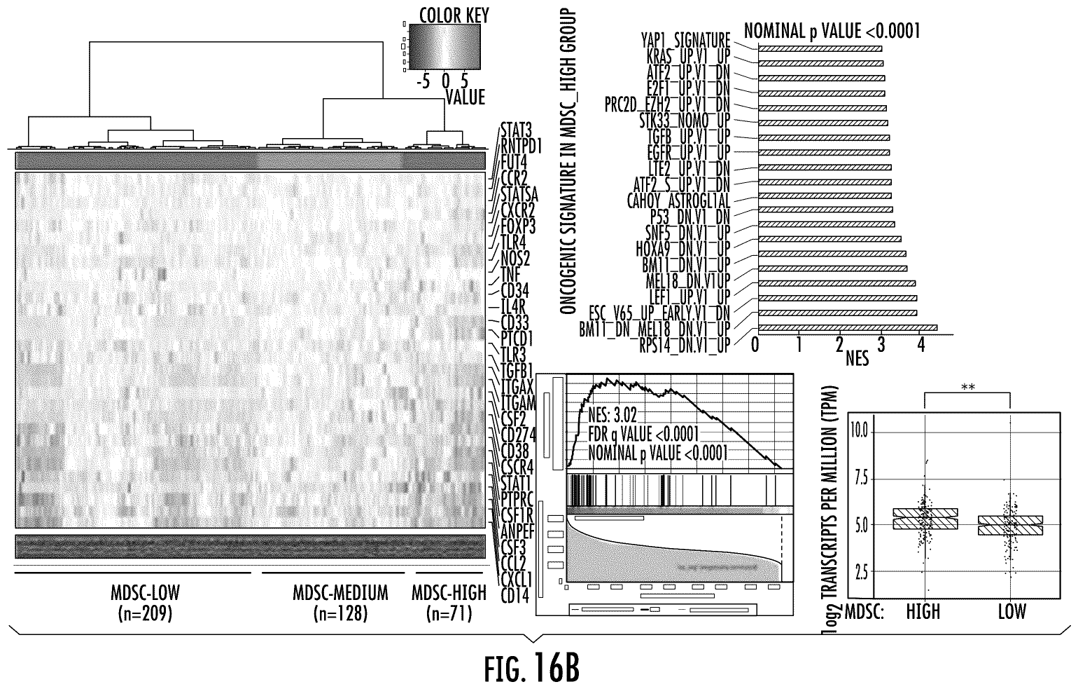

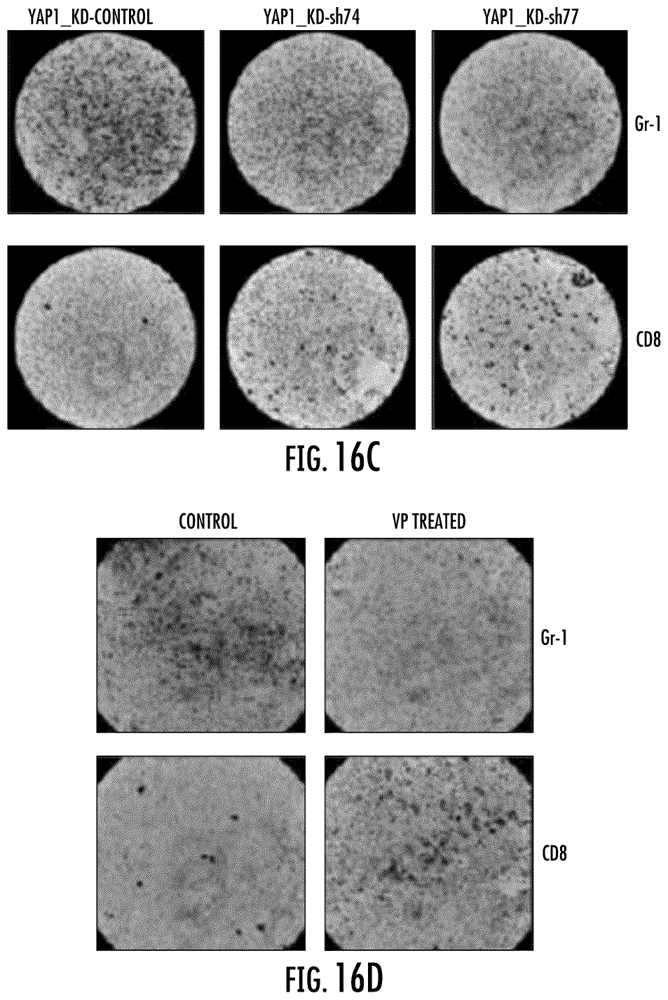

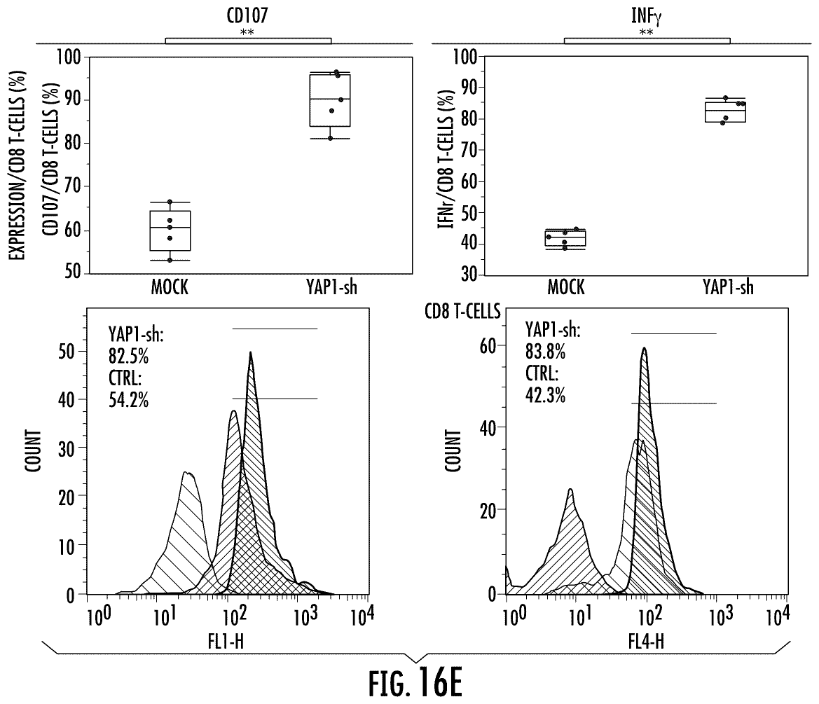

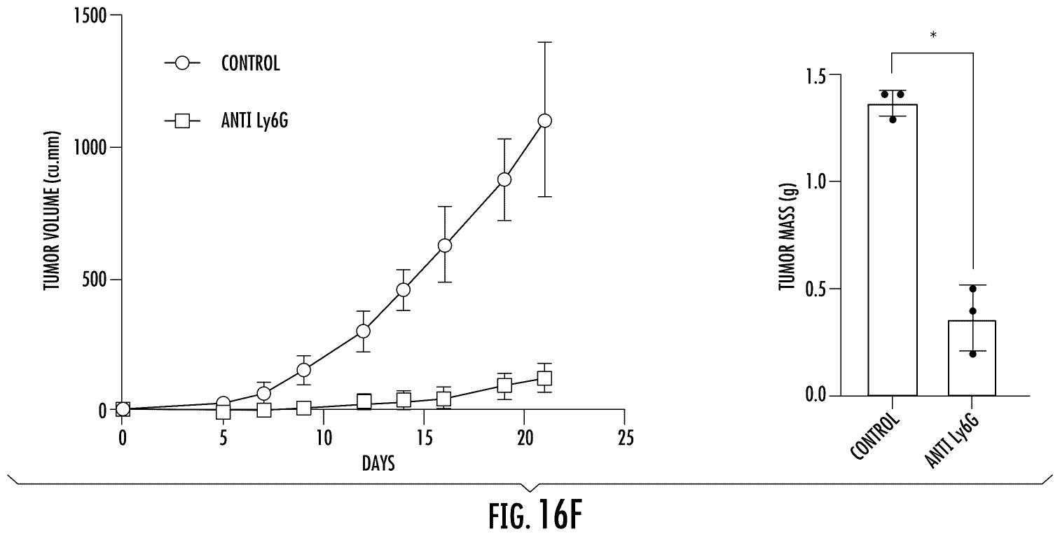

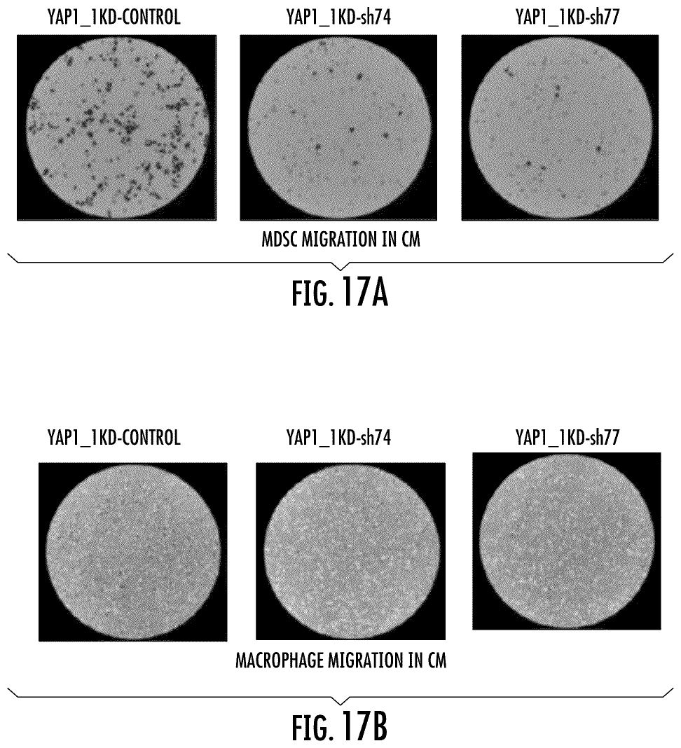

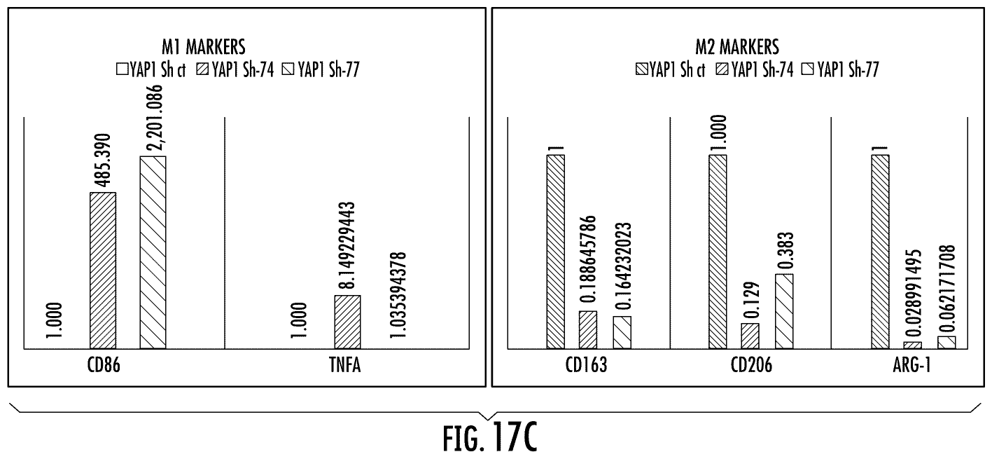

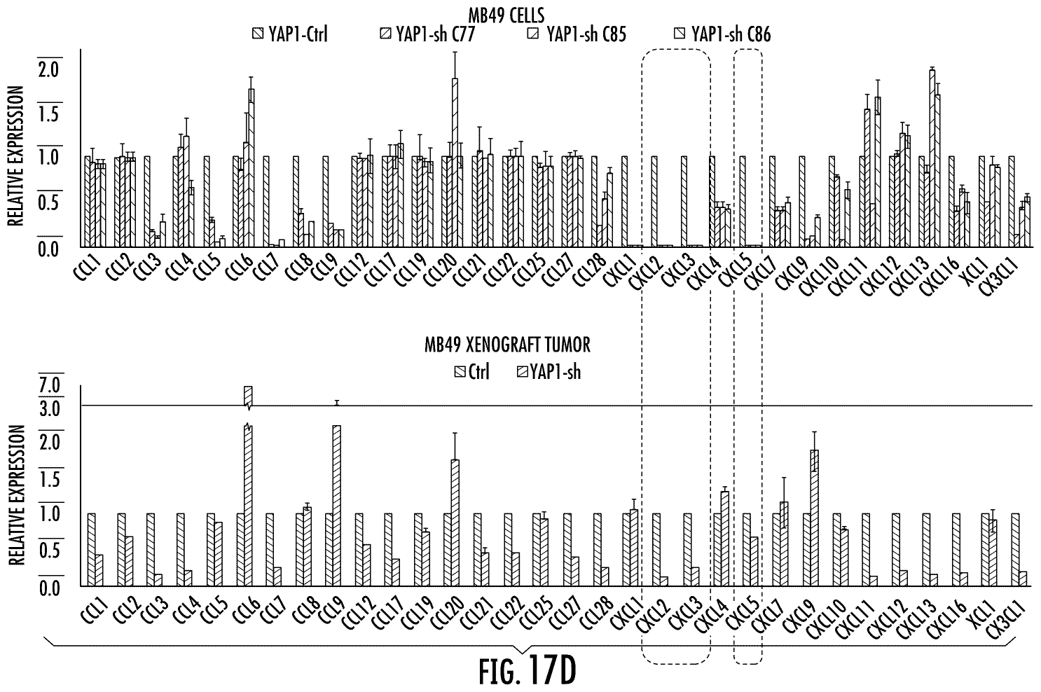

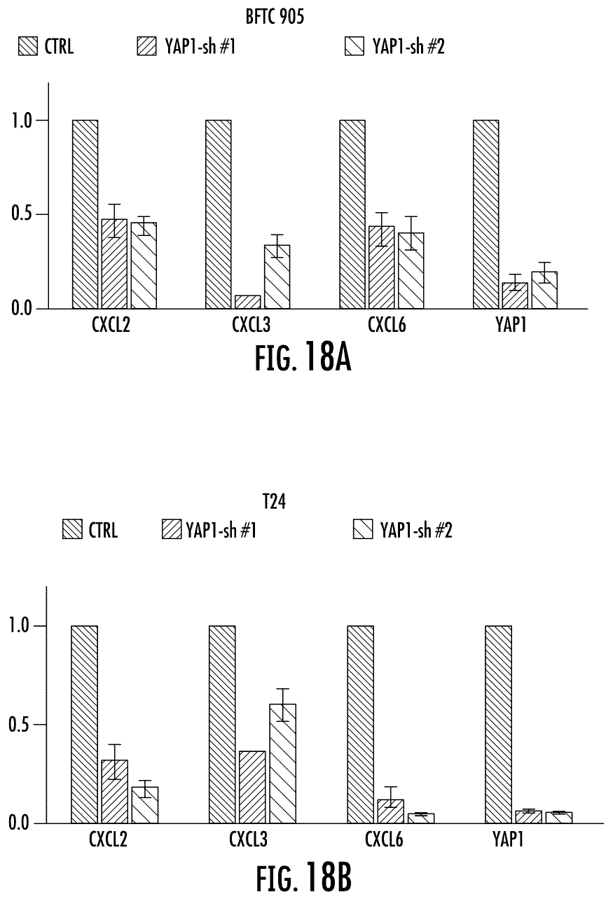

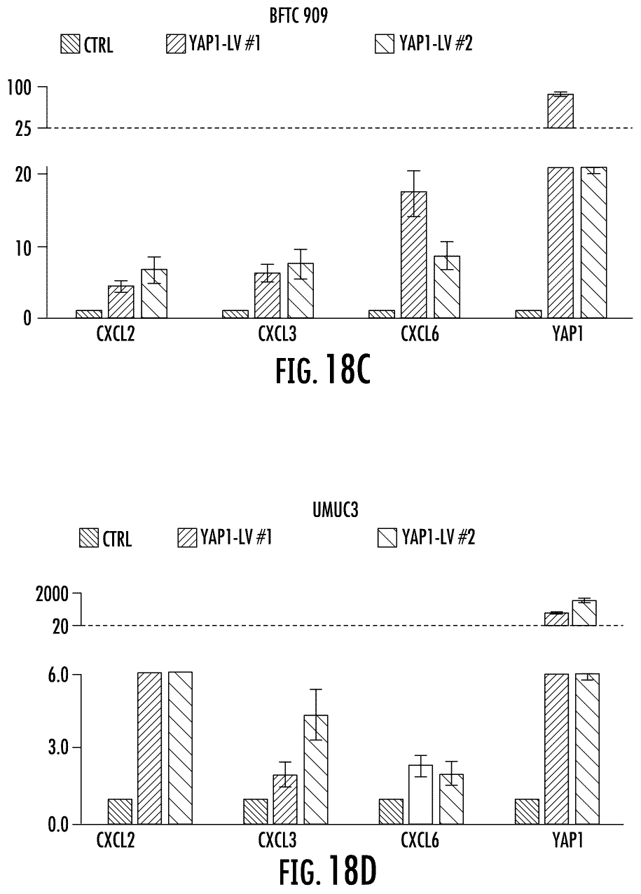

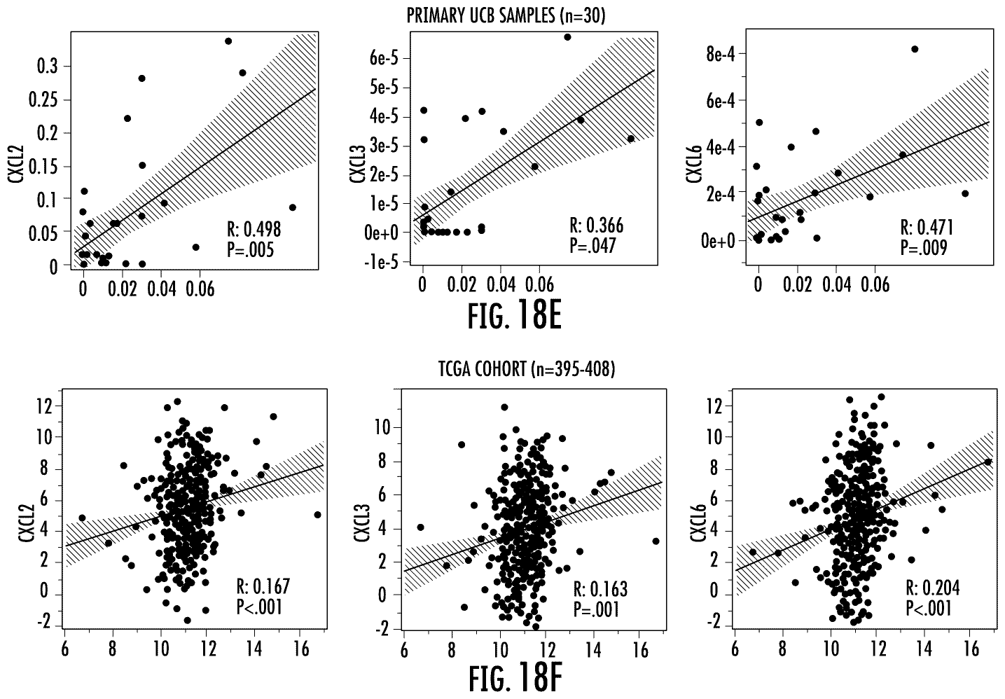

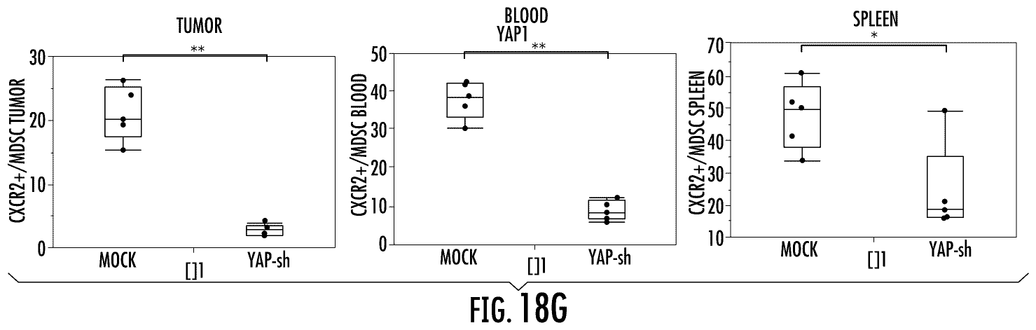

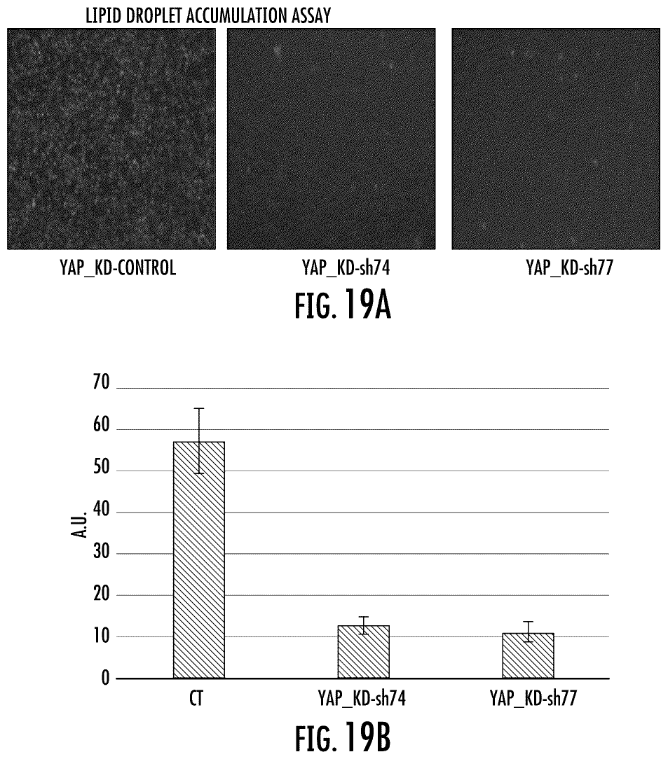

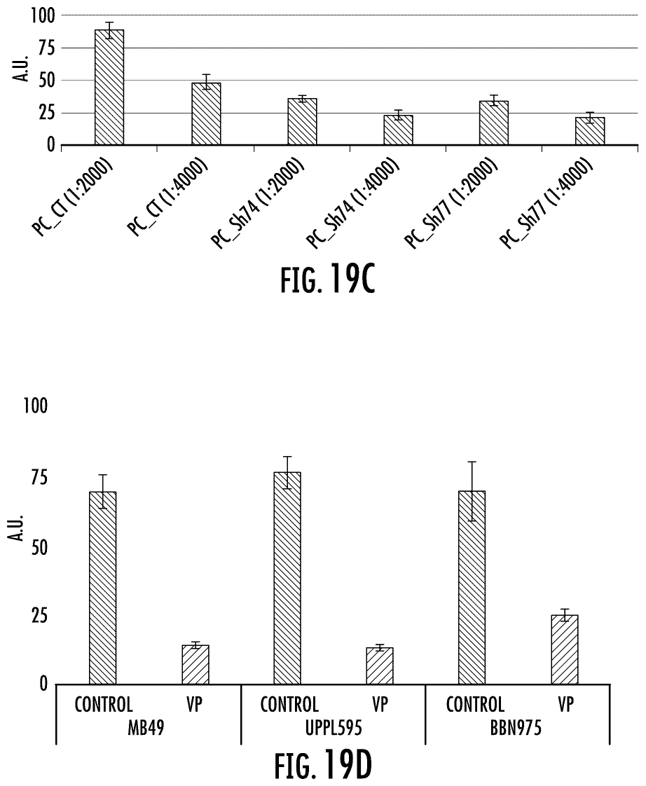

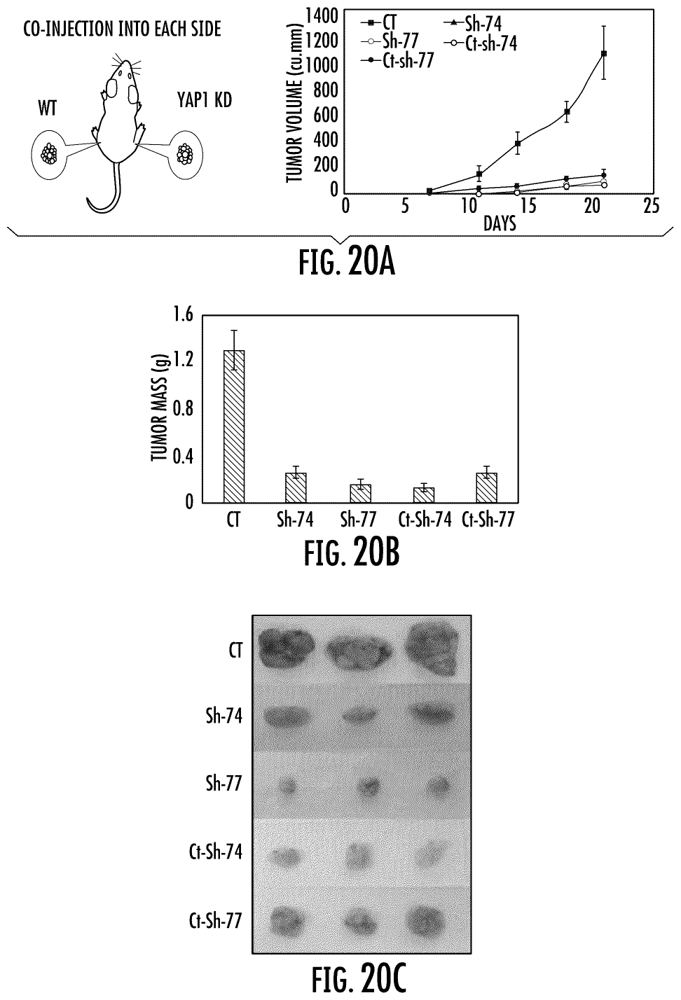

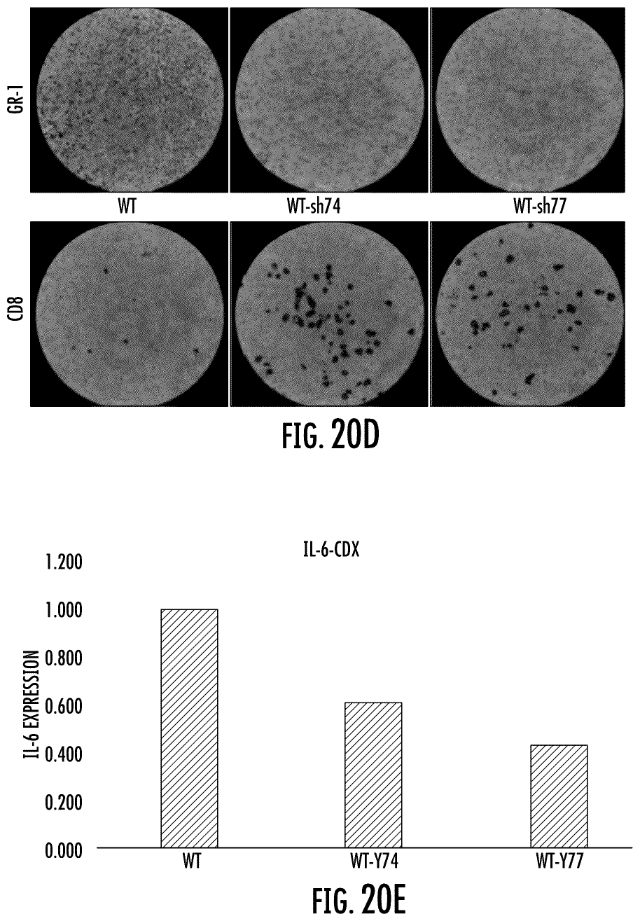

To determine doses of these drugs that are effective for pathway inhibition, H1299 and H1437 cells were treated with verteporfin or S3I-201 for 72 hours. As expected, verteporfin suppressed mRNA expressions of YAP1 and its downstream targets, such as CTGF and CYR61, in a dose-dependent manner (FIG. 11A). Similarly, S3I-201 suppressed both STAT3 and its targeted genes, such as NRP1 and PROS1 (refs. 26-28; FIG. 11B). Notably, immunoblotting showed that both YAP1 and total STAT3 expressions were suppressed after verteporfin treatment (FIG. 5A), whereas total STAT3 expression was not affected in the genetically YAP1-knockdown and -overexpressing cells (FIG. 2A). On the other hand, S3I-201 suppressed pSTAT3 without affecting YAP1 or total STAT3 expressions (FIG. 5B).

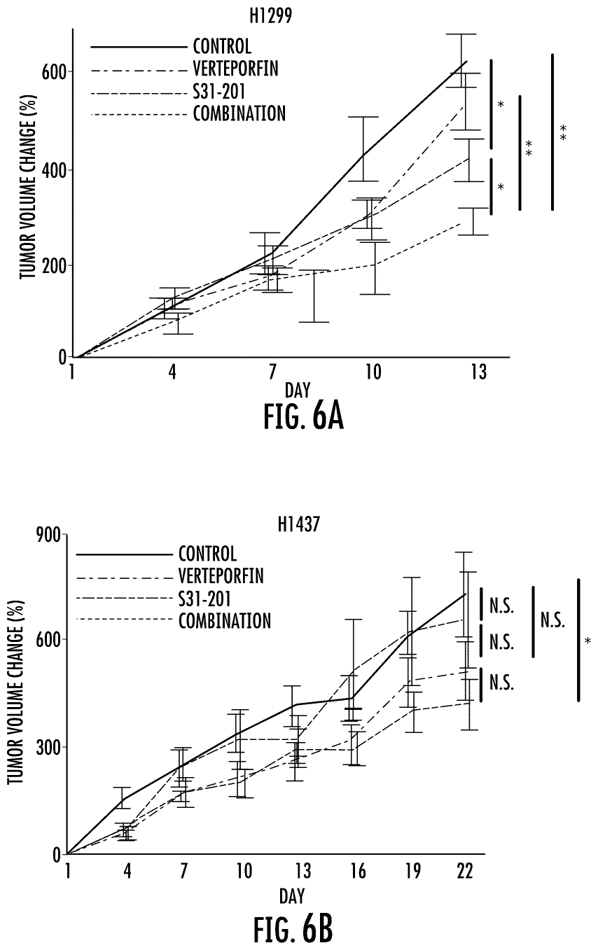

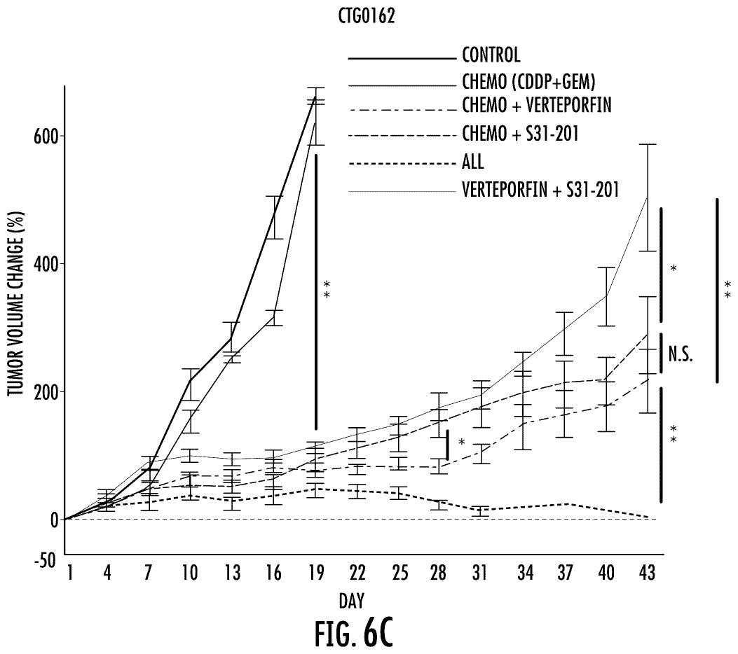

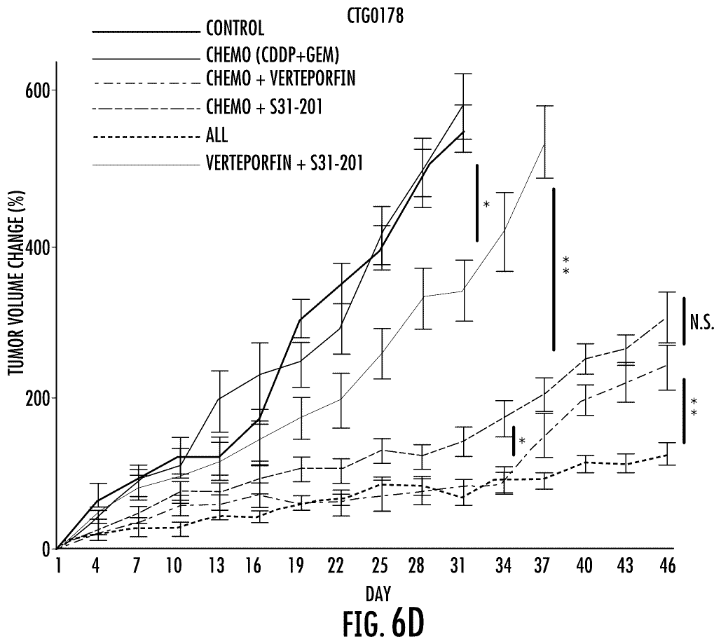

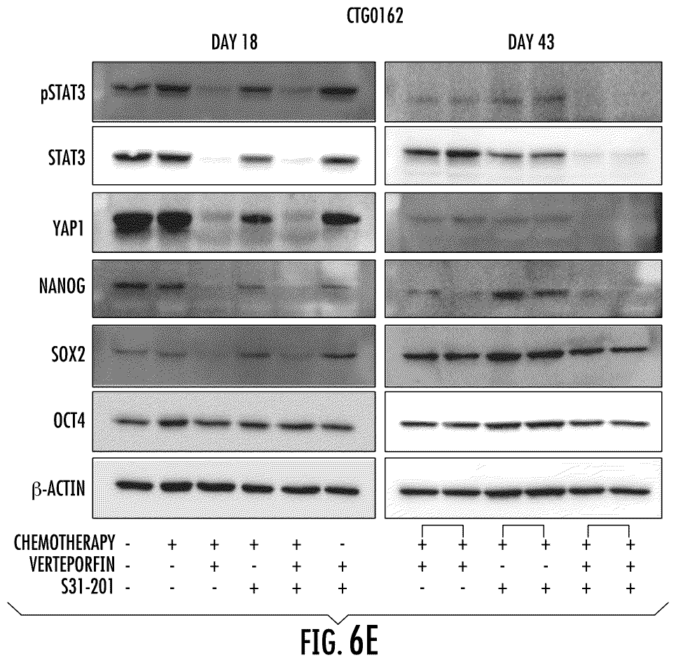

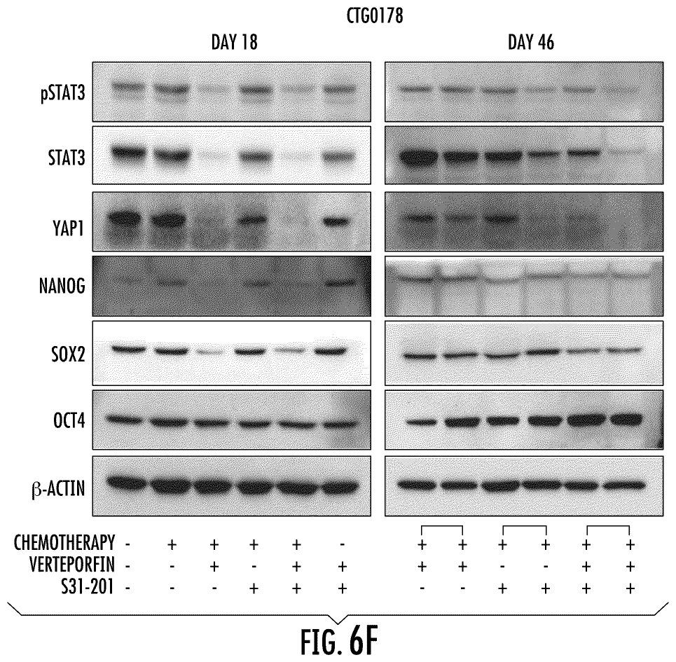

Synergistic tumor growth inhibitory effects of verteporfin and S3I-201 in mice. We assessed therapeutic efficacies of these drugs in cell lines and PDX mouse models. Whereas single agents inhibited tumor growth in H1299 xenografts, combinational treatment resulted in greater growth inhibition (FIG. 6A). In H1437 xenografts, only the combination of verteporfin+S3I-201 showed significant growth inhibition compared with controls (FIG. 6B). Although the combination of drugs was more effective, these drugs did not effectively suppress tumor growth for the long term.

We subsequently treated mice bearing heterogeneous and clinically relevant PDX tumors with a combination of these two agents plus chemotherapy (cisplatin+gemcitabine, one of the standard regimens for lung adenocarcinoma treatment). From 13 PDXs that we evaluated, we selected CTG0162 and CTG0178, which expressed YAP1 and pSTAT3, and were confirmed as EGFR wild-types. Both PDX models (CTG0162 and CTG0178) showed significant and continuous tumor regression in “all” group (treated with cisplatin+gemcitabine+verteporfin+S3I-201) compared with the other regimens (FIGS. 6C and D), which suggests that chemotherapeutic efficacy was markedly increased when combined with YAP1 and STAT3 inhibitory agents.

Pharmacodynamics study using PDX tissues. To determine therapeutic effects at the molecular level, we collected tumors on day 18 and at the end of the treatment from each PDX group. We evaluated protein expressions of YAP1, STAT3, and pSTAT3, and standard cancer stemness factors (NANOG, OCT4, and SOX2; refs. 6, 29-31) in these PDX tissues. Consistent with our in vitro results, YAP1, STAT3, and pSTAT3 were decreased in the tumors resected on day 18 from the chemo+verteporfin and “all” groups. However, over time (day 43 for CTG0162 and day 46 for CTG0178), these protein expressions were increased in chemo+verteporfin group, but not in tumors from the “all” group (FIGS. 6E and F). Interestingly, NANOG and SOX2 were expressed in the chemo group, whereas addition of verteporfin decreased their expressions in tumors examined on day 18 (FIGS. 6E and F). Furthermore, NANOG in CTG0162 and CTG0178 and SOX2 in CTG0178 were suppressed over the long-term in the “all” group compared with the chemo+verteporfin group. These findings indicate that verteporfin reinforces chemotherapeutic efficacy by suppressing both YAP1 and STAT3 and targeting CSCs that evade chemotherapy. Moreover, the addition of S3I-201 to verteporfin could prolong chemotherapeutic efficacy.

### Discussion

Our study showed that dual YAP1 and STAT3 inhibition enhances the efficacy of chemotherapy in EGFR wild-type lung adenocarcinoma, and expressions of pSTAT3 and YAP1 in primary lung adenocarcinoma were significantly positively correlated at protein level. In a recent study, high levels of STAT3 and YAP1mRNA in baseline EGFR mutant NSCLC samples were associated with poor progression-free survival (14). We do not have follow-up data for the cohort of lung adenocarcinoma samples analyzed, and we deliberately selected EGFR wild-type cell lines and PDX models to study the therapeutic efficacy of YAP1 and STAT3 inhibitors with chemotherapy. Future studies with EGFR-mutant cell lines and PDX models are needed to assess the effect of EGFR status on the efficacy of this drug strategy.

On the basis of our cytokine analysis, we hypothesized that STAT3 phosphorylation is regulated by YAP1 through IL6, and showed that YAP1 directly upregulates IL6 to induce STAT3 phosphorylation. Recent studies have reported the association between YAP1 and STAT3 expressions (14, 32, 33) and the role of IL6 to activate STAT3 in NSCLC (34, 35). However, linking YAP1 to IL6 and STAT3 is a novel finding in this study for understanding tumor development and potential drug resistance. We found a positive correlation between mRNA expressions of YAP1 and IL6. Furthermore, our IHC analysis showed a positive correlation between protein expressions of YAP1 and pSTAT3 in patients with lung adenocarcinoma. As cytokines, including IL6, are small secreted proteins that are physiologically present in low concentrations and have highly specific activities (36), no well-validated IHC methods are available for IL6 analysis. Future studies with appropriate antibodies and well-validated IHC assays for IL6 are warranted to explore the YAP1-IL6-pSTAT3 signaling axis at the protein level in primary lung adenocarcinoma. Although STAT3 has been reported to be phosphorylated by several pathways, such as JAK/STAT pathway (10), our data suggested that the YAP1-IL6-pSTAT3 pathway is one of these pathways. We did not perform IHC of total STAT3 in our tumor microenvironment sample to determine whether there is any correlation between pSTAT3 and total STAT3. However, in our cell lines and PDX models data, we did not observe any correlation between pSTAT3 and total SAT3 expressions, which is consistent with the previous study by others (37).

YAP1 helps cancer cells to overcome DNA damage and subsequent inhibition of DNA synthesis induced by chemotherapy (38). As YAP1 is not essential for normal tissue homeostasis in adults (38), YAP1-targeting therapy is considered to be a promising strategy (3). Chemotherapy usually eradicates the bulk of cancer cells, but CSCs can evade therapeutic response, resulting in drug resistance (7). We previously demonstrated that YAP1 upregulates SOX2 to enhance CSC phenotypes with activated COX2/PGE2 signaling in basal-type bladder cancer; and that verteporfin and COX2 inhibitor reinforce chemotherapeutic efficacy by suppressing CSC properties (6). STAT3 also enriches CSC properties, and STAT3 inhibitor combined with carboplatin attenuated stemness-like features in breast cancer (39). These findings suggest that YAP1 and STAT3 independently regulate CSCs and oncogenic pathways (3, 6, 40). Therefore, simultaneous inhibition of YAP1 and STAT3 is expected to enhance chemotherapeutic efficacy by eradicating CSCs. In fact, our results showed that the dual inhibition of YAP1 and STAT3 decreased stem cell-like features, resulting in reduced proliferation and increased sensitivity to cisplatin.

Verteporfin has recently been shown to bind to YAP1 and change its conformation, thereby abrogating its interaction with TEAD2, which suppresses the YAP1 oncogenic function (9, 41). However, a few reports indicate that verteporfin is a promising chemotherapeutic agent, independent of its effect on YAP1 (42, 43). Another off-target effect of verteporfin is the formation of cross-linked oligomers and high molecular weight protein complexes, which are hypothesized to interfere with autophagy and cell growth (44). Currently, we and others did not observe any toxicity associated with these off-target effects in vivo. However, a recent report suggests that verteporfin triggered accumulation of toxic amounts of protein oligomers that selectively killed colorectal cancer cells in mice and in cells cultured under hypoxic and nutrient-deprived conditions; normal cells in culture and in tumor adjacent tissue sections from mice cleared these aggregates through autophagy and survived (45). Our in vitro results showed STAT3 oligomerization upon verteporfin treatment in a dose dependent manner, but we did not notice any toxicity in mice. Therefore, verteporfin can potentially be used clinically after further preclinical validation in animal models. Recently, other than verteporfin, several drugs (e.g., repotrectinib) have been reported to abrogate YAP1 activity (46-48). Future studies will be needed to determine which drug can suppress YAP1 activity effectively with less toxicity.

In treating PDX models, the addition of verteporfin and S3I-201 to conventional chemotherapy dramatically suppressed tumor growth compared with chemotherapy alone. Although initially, chemo+verteporfin showed tumor-suppressive effects similar to those of the “all” regimen, its inhibitory effect subsided at a slower rate. In pharmacodynamics analysis, chemo+verteporfin-treated tumors resected on the last day of treatment expressed greater levels of YAP1, STAT3, NANOG, and SOX2 than the “all” group, which suggests that S3I-201 and verteporfin have synergistic effects in suppressing CSC populations. Notably, the combination of verteporfin+S3I-201 without chemotherapy did not show any tumor-suppressive effects in PDX models and YAP1 and pSTAT3 expressions were not decreased dramatically in this treatment regimen, unlike cell line models. This discrepancy is probably a reflection of tumor heterogeneity in the PDXs, and CSC population remains almost same compared with the controls.

Importantly, verteporfin and STAT3-SH2 domain inhibitors have been used clinically, or in clinical trials, and few severe adverse effects were reported for these inhibitors (49, 50). Therefore, after further preclinical studies, these drugs may be easier to be administered to patients than other new molecule-targeting drugs.

In summary, our study indicates that the combination of YAP1 and STAT3 inhibitor with chemotherapy has promise for the treatment of lung adenocarcinoma. Furthermore, our study strongly demands for further preclinical and clinical studies to develop novel therapeutic strategies for lung adenocarcinoma that overexpress YAP1 and STAT3 and thus, may provide an insight to the clinical significance of the attenuation of CSCs in combating chemotherapeutic resistance.

### REFERENCES

1. Jones C M, Brunelli A, Callister M E, Franks K N. Multimodality treatment of advanced non-small cell lung cancer: where are we with the evidence? Curr Surg Rep 2018; 6:5.
2. Lategahn J, Keul M, Rauh D. Lessons to be learned: themolecular basis of kinase targeted therapies and drug resistance in non-small cell lung cancer. Angew Chem Int Ed Engl 2018; 57:2307-13.
3. Shibata M, Ham K, Hoque M O. A time for YAP1: tumorigenesis, immunosuppression and targeted therapy. Int J Cancer 2018; 143:2133-44.
4. Lee J E, Park H S, Lee D, Yoo G, Kim T, Jeon H, et al. Hippo pathway effector YAP inhibition restores the sensitivity of EGFR-TKI in lung adenocarcinoma having primary or acquired EGFR-TKI resistance. Biochem Biophys Res Commun 2016; 474:154-60.
5. Zhang M, Zeng J, Zhao Z, Liu Z. Loss of MiR-424-3p, not miR-424-5p, confers chemoresistance through targeting YAP1 in non-small cell lung cancer. Mol Carcinog 2017; 56:821-32.
6. Ooki A, Del Carmen Rodriguez Pena M, Marchionni L, Dinalankara W, Begum A, Hahn N M, et al. YAP1 and COX2 coordinately regulate urothelial cancer stem-like cells. Cancer Res 2018; 78:168-81.
7. Shibata M, Hoque M O. Targeting cancer stem cells: a strategy for effective eradication of cancer. Cancers 2019; 11:732.
8. Gibault F, Bailly F, Corvaisier M, Coevoet M, Huet G, Melnyk P, et al. Molecular features of the YAP inhibitor verteporfin: synthesis of hexasubstituted dipyrrins as potential inhibitors of YAP/TAZ, the downstream effectors of the hippo pathway. ChemMedChem 2017; 12:954-61.
9. Liu-Chittenden Y, Huang B, Shim J S, Chen Q, Lee S J, Anders R A, et al. Genetic and pharmacological disruption of the TEAD-YAP complex suppresses the oncogenic activity of YAP. Genes Dev 2012; 26:1300-5.
10. Shien K, Papadimitrakopoulou V A, Ruder D, Behrens C, Shen L, Kalhor N, et al. JAK1/STAT3 activation through a proinflammatory cytokine pathway leads to resistance to molecularly targeted therapy in non-small cell lung cancer. Mol Cancer Ther 2017; 16:2234-45.
11. Tong M, Wang J, Jiang N, Pan H, Li D. Correlation between p-STAT3 overexpression and prognosis in lung cancer: a systematic review and meta-analysis. PLoS One 2017; 12:e0182282.
12. Chai E Z, Shanmugam M K, Arfuso F, Dharmarajan A, Wang C, Kumar A P, et al. Targeting transcription factor STAT3 for cancer prevention and therapy. Pharmacol Ther 2016; 162:86-97.
13. Johnson D E, O'Keefe R A, Grandis J R. Targeting the IL-6/JAK/STAT3 signalling axis in cancer. Nat Rev Clin Oncol 2018; 15:234-48.
14. Chaib I, Karachaliou N, Pilotto S, Codony Servat J, Cai X, Li X, et al. Co-activation of STAT3 and YES-associated protein 1 (YAP1) pathway in EGFR-mutant NSCLC. J Natl Cancer Inst 2017; 109:djx014.
15. Zhang K, Qi H X, Hu Z M, Chang Y N, Shi Z M, Han X H, et al. YAP and TAZ take center stage in cancer. Biochemistry 2015; 54:6555-66.
16. Huynh J, Etemadi N, Hollande F, Ernst M, Buchert M. The JAK/STAT3 axis: a comprehensive drug target for solid malignancies. Semin Cancer Biol 2017; 45: 13-22.
17. Shibata M, Kanda M, Tanaka H, Umeda S, Miwa T, Shimizu D, et al. Overexpression of Derlin 3 is associated with malignant phenotype of breast cancer cells. Oncol Rep 2017; 38:1760-6.
18. Siddiquee K, Zhang S, Guida W C, Blaskovich M A, Greedy B, Lawrence H R, et al. Selective chemical probe inhibitor of Stat3, identified through structure-based virtual screening, induces antitumor activity. Proc Natl Acad Sci USA 2007; 104: 7391-6.
19. Kim T, Yang S J, Hwang D, Song J, Kim M, Kyum Kim S, et al. A basal-like breast cancer-specific role for SRF-IL6 in YAP-induced cancer stemness. Nat Commun 2015; 6:10186.
20. Ooki A, Maleki Z, Tsay J J, Goparaju C, Brait M, Turaga N, et al. A panel of novel detection and prognostic methylated DNA markers in primary non-small cell lung cancer and serum DNA. Clin Cancer Res 2017; 23:7141-52.
21. Munari E, Zamboni G, Lunardi G, Marchionni L, Marconi M, Sommaggio M, et al. PD-L1 expression heterogeneity in non-small cell lung cancer: defining criteria for harmonization between biopsies and whole sections. J Thorac Oncol 2018; 13:1113-20.
22. Dong X, Guan J, English J C, Flint J, Yee J, Evans K, et al. Patient-derived first generation xenografts of non-small cell lung cancers: promising tools for predicting drug responses for personalized chemotherapy. Clin Cancer Res 2010; 16:1442-51.
23. Mir S A, Chatterjee A, Mitra A, Pathak K, Mahata S K, Sarkar S. Inhibition of signal transducer and activator of transcription 3 (STAT3) attenuates interleukin-6 (IL-6)-induced collagen synthesis and resultant hypertrophy in rat heart. J Biol Chem 2012; 287:2666-77.
24. Gruber R, Panayiotou R, Nye E, Spencer-Dene B, Stamp G, Behrens A. YAP1 and TAZ control pancreatic cancer initiation in mice by direct up-regulation of JAKSTAT3 signaling. Gastroenterology 2016; 151:526-39.
25. Roskoski R Jr. Janus kinase (JAK) inhibitors in the treatment of inflammatory and neoplastic diseases. Pharmacol Res 2016; 111:784-803.
26. de Wolf C J, Cupers R M, Bertina R M, Vos H L. Interleukin-6 induction of protein s is regulated through signal transducer and activator of transcription 3. Arterioscler Thromb Vasc Biol 2006; 26:2168-74.
27. Wong A L A, Hirpara J L, Pervaiz S, Eu J Q, Sethi G, Goh B C. Do STAT3 inhibitors have potential in the future for cancer therapy? Expert Opin Investig Drugs 2017; 26:883-7.
28. Zhang S, Zhau H E, Osunkoya A O, Iqbal S, Yang X, Fan S, et al. Vascular endothelial growth factor regulates myeloid cell leukemia-1 expression through neuropilin-1-dependent activation of c-MET signaling in human prostate cancer cells. Mol Cancer 2010; 9:9.
29. Bora-Singhal N, Nguyen J, Schaal C, Perumal D, Singh S, Coppola D, et al. YAP1 regulates OCT4 activity and SOX2 expression to facilitate self-renewal and vascular mimicry of stem-like cells. Stem Cells 2015; 33:1705-18.
30. Do D V, Ueda J, Messerschmidt D M, Lorthongpanich C, Zhou Y, Feng B, et al. A genetic and developmental pathway from STAT3 to the OCT4-NANOG circuit is essential for maintenance of ICM lineages in vivo. Genes Dev 2013; 27:1378-90.
31. Wang H, Deng J, Ren H Y, Jia P, Zhang W, Li M Q, et al. STAT3 influences the characteristics of stem cells in cervical carcinoma. Oncol Lett 2017; 14:2131-6.
32. Taniguchi K, Moroishi T, de Jong P R, Krawczyk M, Grebbin B M, Luo H, et al. YAP-IL-6ST autoregulatory loop activated on APC loss controls colonic tumorigenesis. Proc Natl Acad Sci USA 2017; 114:1643-8.
33. Zhu M, Liu X, Wang Y, Chen L, Wang L, Qin X, et al. YAP via interacting with STAT3 regulates VEGF-induced angiogenesis in human retinal microvascular endothelial cells. Exp Cell Res 2018; 373:155-63.
34. Brooks G D, McLeod L, Alhayyani S, Miller A, Russell P A, Ferlin W, et al. IL6 trans-signaling promotes KRAS-driven lung carcinogenesis. Cancer Res 2016; 76:866-76.
35. Gao S P, Mark K G, Leslie K, Pao W, Motoi N, Gerald W L, et al. Mutations in the EGFR kinase domain mediate STAT3 activation via IL-6 production in human lung adenocarcinomas. J Clin Invest 2007; 117:3846-56.
36. Vitkovic L, Bockaert J, Jacque C. “Inflammatory” cytokines: neuromodulators in normal brain? J Neurochem 2000; 74:457-71.
37. Park J H, van Wyk H, McMillan D C, Quinn J, Clark J, Roxburgh C S, et al. Signal transduction and activator of transcription-3 (STAT3) in patients with colorectal cancer: associations with the phenotypic features of the tumor and host. Clin Cancer Res 2017; 23:1698-709.
38. Lo Sardo F, Strano S, Blandino G. YAP and TAZ in lung cancer: oncogenic role and clinical targeting. Cancers 2018; 10:137.
39. Lu H, Chen I, Shimoda L A, Park Y, Zhang C, Tran L, et al. Chemotherapy induced Ca(2+) release stimulates breast cancer stem cell enrichment. Cell Rep 2017; 18:1946-57.
40. Galoczova M, Coates P, Vojtesek B. STAT3, stem cells, cancer stem cells and p63. Cell Mol Biol Lett 2018; 23:12.
41. Kandoussi I, Lakhlili W, Taoufik J, Ibrahimi A. Docking analysis of verteporfin with YAP WW domain. Bioinformation 2017; 13:237-40.
42. Dasari V R, Mazack V, Feng W, Nash J, Carey D J, Gogoi R. Verteporfin exhibits YAP-independent anti-proliferative and cytotoxic effects in endometrial cancer cells. Oncotarget 2017; 8:28628-40.
43. Eales K L, Wilkinson E A, Cruickshank G, Tucker J H R, Tennant D A. Verteporfin selectively kills hypoxic glioma cells through iron-binding and increased production of reactive oxygen species. Sci Rep 2018; 8:14358.
44. Konstantinou E K, Notomi S, Kosmidou C, Brodowska K, Al-Moujahed A, Nicolaou F, et al. Verteporfin-induced formation of protein cross-linked oligomers and high molecular weight complexes is mediated by light and leads to cell toxicity. Sci Rep 2017; 7:46581.
45. Zhang H, Ramakrishnan S K, Triner D, Centofanti B, Maitra D, Gyorffy B, et al. Tumor-selective proteotoxicity of verteporfin inhibits colon cancer progression independently of YAP1. Sci Signal 2015; 8:ra98.
46. Karachaliou N, Chaib I, Cardona A F, Berenguer J, Bracht J W P, Yang J, et al. Common co-activation of AXL and CDCP1 in EGFR-mutation-positive nonsmall cell lung cancer associated with poor prognosis. EBioMedicine 2018; 29: 112-27.
47. Kurppa K J, Liu Y, To C, Zhang T, Fan M, Vajdi A, et al. Treatment-induced tumor dormancy through YAP-mediated transcriptional reprogramming of the apoptotic pathway. Cancer Cell 2020; 37:104-22.
48. Yun M R, Choi H M, Lee Y W, Joo H S, Park C W, Choi J W, et al. Targeting YAP to overcome acquired resistance to ALK inhibitors in ALK-rearranged lung cancer. EMBO Mol Med 2019; 11:e10581.
49. Okusaka T, Ueno H, Ikeda M, Mitsunaga S, Ozaka M, Ishii H, et al. Phase 1 and pharmacological trial of OPB-31121, a signal transducer and activator of transcription-3 inhibitor, in patients with advanced hepatocellular carcinoma. Hepatol Res 2015; 45:1283-91.
50. Wong A L, Soo R A, Tan D S, Lee S C, Lim J S, Marban P C, et al. Phase I and biomarker study of OPB-51602, a novel signal transducer and activator of transcription (STAT) 3 inhibitor, in patients with refractory solid malignancies. Ann Oncol 2015; 26:998-1005.

TABLE 1
Sequences of primers for qRT-PCR used in the present study.

| Gene | | | Annealing |
|---|---|---|---|
| Symbol | Forward Primer | Reverse Primer | Temperature |
| β-actin | TTCTACAATGAGCTGCGTGT | GGGGTGTTGAAGGTCTCAA | 58 |
| | G | A | |
| | (SEQ ID NO: 5) | (SEQ ID NO: 6) | |
| ABCG2 | AGCTGCAAGGAAAGATCCA | TCCAGACACACCACGGATA | 58 |
| | A | A | |
| | (SEQ ID NO: 7) | (SEQ ID NO: 8) | |
| ALDH1A1 | TGTTAGCTGATGCCGACTTG | TTCTTAGCCCGCTCAACACT | 58 |
| | (SEQ ID NO: 9) | (SEQ ID NO: 10) | |
| CD24 | CTGGCACTGCTCCTAC | GAGTGAGACCACGAAG | 58 |
| | (SEQ ID NO: 11) | (SEQ ID NO: 12) | |
| CTGF | CCTGGTCCAGACCACAGAG | TGGAGATTTTGGGAGTACGG | 58 |
| | T | (SEQ ID NO: 14) | |
| | (SEQ ID NO: 13) | | |
| CYR61 | CTCCCTGTTTTTGGAATGGA | TGGTCTTGCTGCATTTCTTG | 58 |
| | (SEQ ID NO: 15) | (SEQ ID NO: 16) | |
| EGF | CAGGGAAGATGACCACCAC | CAGTTCCCACCACTTCAGGT | 58 |
| | T | (SEQ ID NO: 18) | |
| | (SEQ ID NO: 17) | | |
| IFN-γ | ATGTATTGCTTTGCGTTGGA | TCAATAGCAACAAAAAGAA | 58 |
| | CA | ACGAGAT | |
| | (SEQ ID NO: 19) | (SEQ ID NO: 20) | |
| IL-2 | ACCTCAACTCCTGCCACAA | GCCTTCTTGGGCATGTAAAA | 58 |
| | T | (SEQ ID NO: 22) | |
| | (SEQ ID NO: 21) | | |
| IL-4 | GGCAGTTCTACAGCCACCA | CTCTGGTTGGCTTCCTTCAC | 58 |
| | T | (SEQ ID NO: 24) | |
| | (SEQ ID NO: 23) | | |
| IL-6 | ATGTAGCCGCCCCACACAG | CATCCATCTTTTTCAGCCAT | 58 |
| | A | (SEQ ID NO: 26) | |
| | (SEQ ID NO: 25) | | |
| IL-10 | TGCAAAACCAAACCACAAG | TCTCGGAGATCTCGAAGCAT | 58 |
| | A | (SEQ ID NO: 28) | |
| | (SEQ ID NO: 27) | | |
| NANOG | CAGCTGTGTGTACTCAATG | ACACCATTGCTATTCTTCGG | 58 |
| | ATAGATTT | CCAGTTG | |
| | (SEQ ID NO: 29) | (SEQ ID NO: 30) | |
| NRP1 | GGGAACTTGGTGGATGAAT | TCTATGACCGTGGGCTTTTC | 58 |
| | G | (SEQ ID NO: 32) | |
| | (SEQ ID NO: 31) | | |
| OCT4 | GTCCGAGTGTGGTTCTGTA | CTCAGTTTGAATGCATGGGA | 58 |
| | (SEQ ID NO: 33) | (SEQ ID NO: 34) | |
| PROS1 | TCCTGGTTCTGGAATTGCTC | ACCAAGGCAAGCATAACAC | 58 |
| | (SEQ ID NO: 35) | C | |
| | | (SEQ ID NO: 36) | |
| SOX2 | CCCACCTACAGCATGTCCTA | TGGAGTGGGAGGAAGAGGT | 58 |
| | CTC | AAC | |
| | (SEQ ID NO: 37) | (SEQ ID NO: 38) | |
| STAT3 | ATCACGCCTTCTACAGACTG | CATCCTGGAGATTCTCTACC | 58 |
| | C | ACT | |
| | (SEQ ID NO: 39) | (SEQ ID NO: 40) | |
| TGF-β | CCCAGCATCTGCAAAGCTC | GTCAATGTACAGCTGCCGCA | 58 |
| | (SEQ ID NO: 41) | (SEQ ID NO: 42) | |
| YAP1 | ACCCACAGCTCAGCATCTT | TGGCTTGTTCCCATCCATCA | 58 |
| | CG | G | |
| | (SEQ ID NO: 43) | (SEQ ID NO: 44) | |

### Example 2: Yap1 Induces Bladder Cancer Progression and Promotes Immune Evasion Through IL-6/Stat3 Pathway and Cxcls Deregulation

The Hippo signaling pathways in tumorigenesis were studied in different cancer types but it is still unclear about the role of Hippo “effector” YAP1 on the tumor immune microenvironment (TIME). Here, the present inventors have investigated the effect of YAP1 deregulation on TIME in urothelial carcinoma of bladder (UCB) and evaluated the immunotherapy efficacy with or without YAP1 blocking. Several in vitro and in vivo experiments were performed to determine the role of YAP1 using genetic and pharmacological attenuation of YAP1 activity. The present inventors further analyzed several human samples sets to explore TIME in context with YAP1 expression. Briefly, RNA sequencing was carried out with mice and human cell lines with different level of YAP1 expression to find out YAP1 regulated novel downstream targets in an unbiased manner and experimentally confirmed that YAP1 regulate TIME through IL-6/STAT3 pathway and CXCLs deregulation. The influence of conditioned media and secreted extracellular vesicles (EVs) from YAP1 knockdown (KD) and YAP1 expressed cancer cells were evaluated on macrophage cell lines. The present inventors' results indicate that YAP1KD cells attract less macrophages and MDSC compared to YAP1 expressed cells. In consistent with in vitro data, the present inventors' in vivo findings support that YAP1 KD increases adaptive immune response characterize by developing an exclusive animal experiment using both WT and YAP1 KD MB 49 cells. Furthermore, the therapeutic efficacy of YAP1 attenuation with immunotherapy indicate that targeting YAP1 combination with immunotherapy may have significant clinical values for treating UCB patients. Overall, the present inventors' study provides a comprehensive insight on how YAP1 signaling drives cancer stemness and induce immunosuppressive tumor microenvironment (TME) by influencing the infiltration of MDSCs and polarization of the macrophages.

### Materials and Methods

Cell lines and mice. MB49, UPPL1595 and BBN975 cells were used in this study. MB49 cells are urothelial carcinoma lines derived from C57BL/6 mice by exposure of primary bladder epithelial cell explants to dimethylbenz (a) anthracene (DMBA). The syngeneic, murine models of bladder cancer have been widely used. MB49 cells were maintained in DMEM medium (Mediatech, Manassas, VA, USA) with 10% fetal bovine serum (Hyclone, Logan, UT, USA), respectively, under a 5% CO2 atmosphere at 95% relative humidity. BBN975, UPPL595??

C57BL/6 mice were obtained from Charles River Laboratories (Frederick, USA). NSG mice (immune compromised) were obtained from Johns Hopkins University. Mice were maintained under pathogen-free conditions within the Johns Hopkins Medical Institutes animal care facility in accordance with the American Association of Laboratory Animal Care guidelines.

Cell viability assay. Cell proliferation and viability were evaluated using alamarBlue™ Cell Viability Reagent (ThermoFischer Scientific). For cell proliferation assay, after cells (5×103/well) were seeded into 96-well plates with culture media containing 10% FBS, the optical density of each well was measured at desired time interval, following the manufacturers protocol. The absorbance was measured by a Spectra Max 250 Plate Reader (Molecular Devices). Cell viability were calculated as percentage over control.

Sphere formation assay. Sphere formation was induced by culturing cells (2×104/well) in DMEM/Ham's F12 50/50 Mix (Mediatech) supplemented with B-27 (Life Technologies), 20 ng/mL FGF-basic (Peprotech), 20 ng/mL EGF (Peprotech). Cell culture was performed in ultra-low attachment 6 well plates (Corning, Lowell, USA) for 10 days. The medium was replaced every other day. Sphere formation was evaluated using the inverted phase-contrast microscope.

The cytotoxic T-lymphocyte assay (CTL assay). MB49 YAP1 KD clones (YAP1_Sh-Ct, Sh-74, Sh-77) were plated in a 96 well plate. Adherent tumor cells including. After overnight incubation, activated CD8+ T cells were added to each well at a ratio of 1:1, 1:5 and 1:10 (MB49: CD8+ T cells) and incubated for 16 h. At the end of incubation, the plates were centrifuged at 400 g, 5 min the supernatants in each group were collected for LDH release assay (CytoSelect™ LDH Cytotoxicity Assay Kit) according to the manufacturer's instructions. The absorbance was detected at 490 nm using a Spectra Max 250 Plate Reader (Molecular Devices).

Macrophage isolation and migration assay. Eight-week-old C57Bl/6 mice were intraperitoneally injected with 3% Brewer thioglycollate medium and intraperitoneal macrophages were harvested 3 days after treatment. Cell migration assay was performed using a transwell coculture system in 24-well plates (Corning). For the cell migration assay, primary macrophages were seeded at a density of 1.0×106 cells/well to top wells with an 8.0-mm pore size, cocultured with conditioned media (CM) from cultured MB49 YAP1 KD clones (YAP1_Sh-Ct, Sh-74, Sh-7) in bottom wells for 24 hours, and migrated cells were stained and counted.

MDSC isolation and migration assay. MDSCs were isolated from the spleens of tumor bearing mice using a Mouse MDSC Isolation Kit (Miltenyi Biotec, Cat #130-094-538) and plated in RPMI1640 supplemented with 10% FBS and antibiotics. MDSCs (1×105 cells/well) were seeded in the top chamber of the transwell (Corning). Conditioned media (CM) from cultured MB49 YAP1 KD clones (YAP1_Sh-Ct, Sh-74, Sh-77) were collected and added to the bottom layer of the transwell. After 4 hr incubation, cells that had completely migrated to the bottom chamber were counted.

ELISA. Cells (2×106/100 mm dish) were cultured for 24 hr. Media were removed and replaced with 10 ml serum-free DMEM. Supernatants were collected 24 hr later with any floating cells removed by 0.45 mm filtration. All experiments were performed according to the manufacturer's instructions.

Immunohistochemistry. Tumor issues from mouse models were collected and fixed in 10% formalin overnight. The formalin fixed tissue were transferred to the internal core facility, Johns Hopkins University. Standard protocol was followed to prepare slides using antibodies for Gr-1 (MDSC marker) and CD8+ T cells. The slides were micrographed under 40×C magnification using, MICROSCOPE name.

In vivo xenograft assay and treatment. For in vivo xenograft, cells were suspended in 100 μL of a 1:1 mixture of serum-free DMEM and Cultrex Stem Cell Qualified Reduced Growth Factor Basement Membrane Extract (Trevigen, Gaithersburg, USA), and then injected subcutaneously into the both flanks of C57BL/6 mice for MB49/UPPL595/BBN975 cells.

For treatment, animals will be randomized to different treatment groups of 5 mice after growth of tumors to 100 mm3. The potential drugs are anti-PD-L1 monoclonal antibody (B7-H1, Bio X Cell, West Lebanon, USA) and verteporfin (NedKoo, Morrisville, USA). Verteporfin (50 mg/kg) will be administered via i.p. every other day. Anti-PD-L1 monoclonal antibody and the corresponding isotype antibody (200 μg/mouse) were administered via ip injection every 3 to 4 days. Treatment was performed for 14 days.

All experiments using mice were approved by the Johns Hopkins University Animal Care and Use Committee, and the mice were maintained in accordance with the American Association of Laboratory Animal Care guidelines.

Isolation of immunocytes from mouse organs. Tumors were minced and digested with collagenase type IV (Sigma-Aldrich), hyaluronidase (Sigma-Aldrich), and DNase type IV (Sigma-Aldrich) into HBSS, followed by depletion of red blood cells (RBCs) using ACK lysing buffer (Quality Biological, Gaithersburg, USA). Discontinuous (44% and 67%) Percoll PLUS (GE Healthcare) separation method was used to enrich immunocytes. Bone marrow were collected from tibias and femurs. Whole blood was collected from right ventricle. Excised spleen was smashed on 70 μm cell strainer. The RBCs in blood, bone marrow, and spleen were lysed with ACK lysing buffer. CD11b+Ly6Ghigh MDSCs and CD8+ T-cells were isolated using Myeloid-Derived Suppressor Cell Isolation Kit and CD8a+ T Cell Isolation Kit (Miltenyi Biotec), respectively, according to the manufacturer's instructions.

Isolation and quantification of Extracellular vesicles. MB49 YAP1 KD clones (MB49 YAP1 sh-ct, Y74 and Y77) were seeded in 150 mm culture plate and incubated in DMEM supplemented with 10% Exosome-Depleted FBS (Thermo Fisher) for 24 hr at 37° C. to allow cell attachment. The cells were then washed with PBS twice, and culture medium was switched to 35 mL of DMEM without serum. After incubation for 48 hr, conditioned medium was collected and centrifuged at 2,000 g for 10 min at 4° C. to thoroughly remove cell debris. The resulting supernatant was then filtered through a 0.22 mm PVDF filter (Millipore, #SLGV033RB) to remove cell debris and microvesicles. The flow-through was transferred into ultracentrifuge tubes (BECKMAN COULTER, #344058) and then ultracentrifuged in a Beckman SW32Ti rotor at 30,000 rpm for 90 min at 4° C. The resulting pellets were washed with 35 mL of ice-cold PBS and then ultracentrifuged again at 30,000 rpm for 90 min at 4° C. The resulting EV pellets were re-suspended in ice cold PBS for experimental use. Protein concentrations of EVs were determined using Micro BCA Protein Assay Kit (Thermo, #23235). Nanoparticle tracking analysis was performed using NanoSight NS300 system (Malvern Instruments, Ranch Cucamonga, CA, USA) on isolated EVs diluted 5,000-fold with PBS for analysis.

RNA extraction and quantitative reverse transcriptase polymerase chain reaction (Q-RT-PCR). Total RNA from cell lines was isolated using the RNeasy Plus Mini Kit (Qiagen, Germantown, USA), according to the manufacturer's protocol. This total RNA was converted to cDNA using the SuperScript III First-Strand Synthesis System (Life technologies, Carlsbad, USA), which was then used as a template for qRT-PCR. qRT-PCR was performed using the Fast SYBR Green Master Mix (Thermo Fisher Scientific, Waltham, USA) on a Quant studio 6 Fast Real-Time PCR System (Life technologies) in triplicate. Primer sequences and the thermal cycling conditions were shown in the table below. SDS software (Applied Biosystems) was used to determine cycle threshold (Ct) values. The expression levels were quantified relative to β-actin using the 2−ΔΔCt method.

### Results

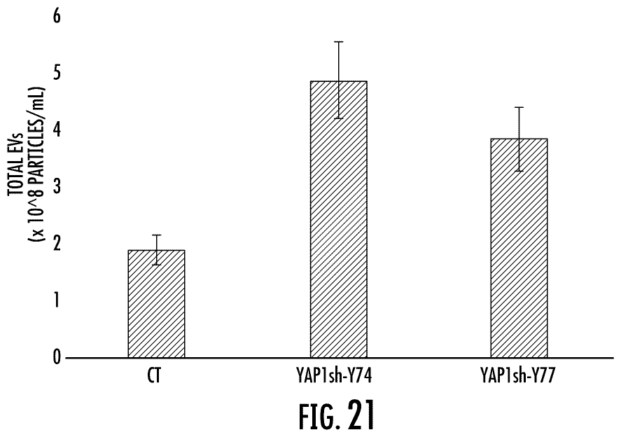

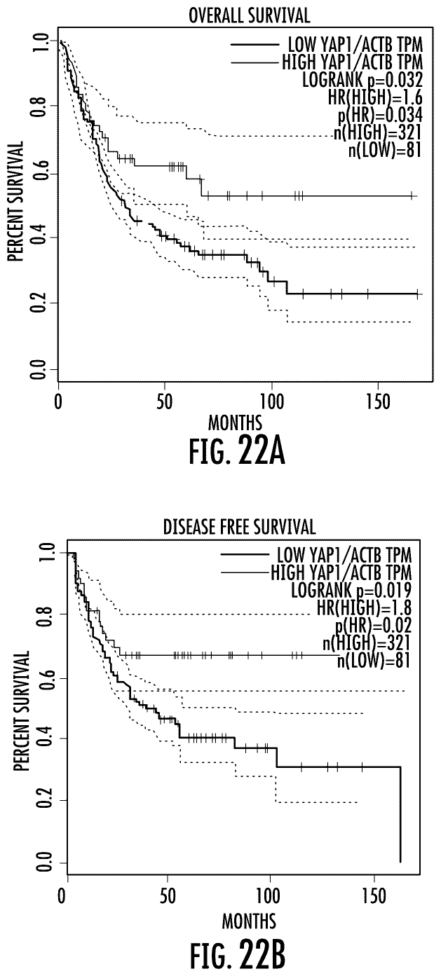

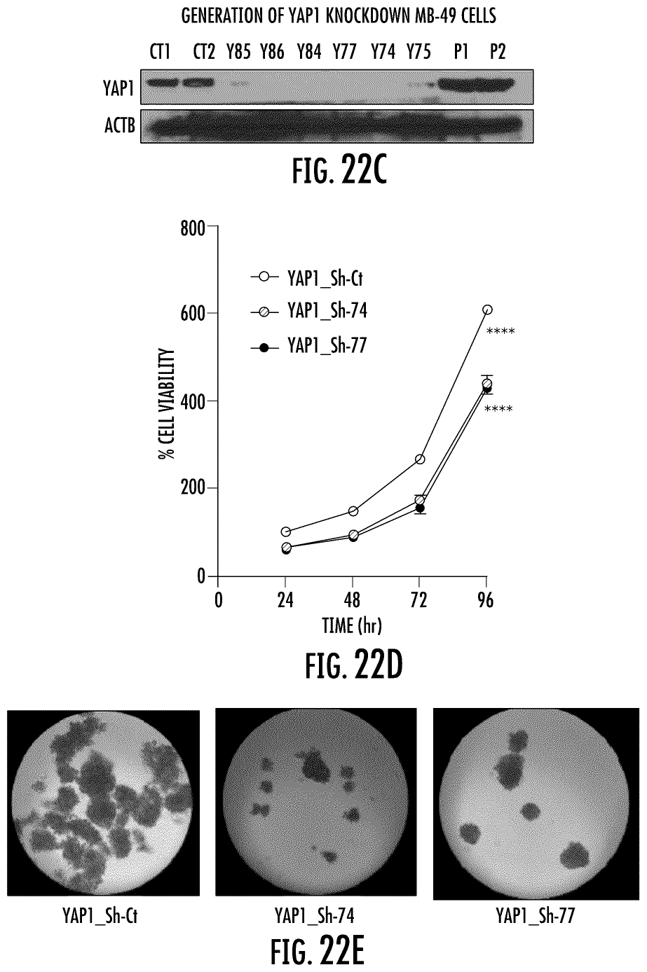

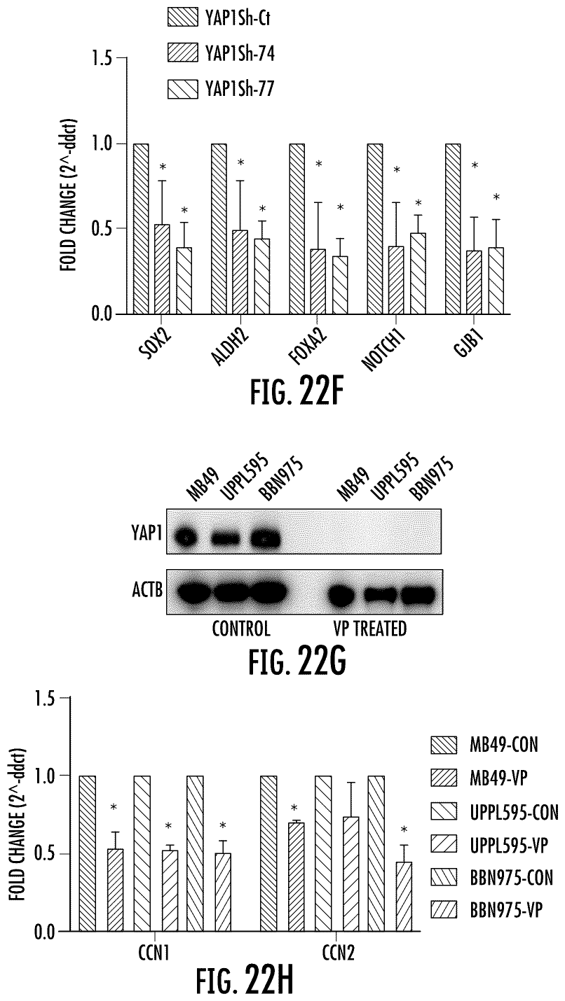

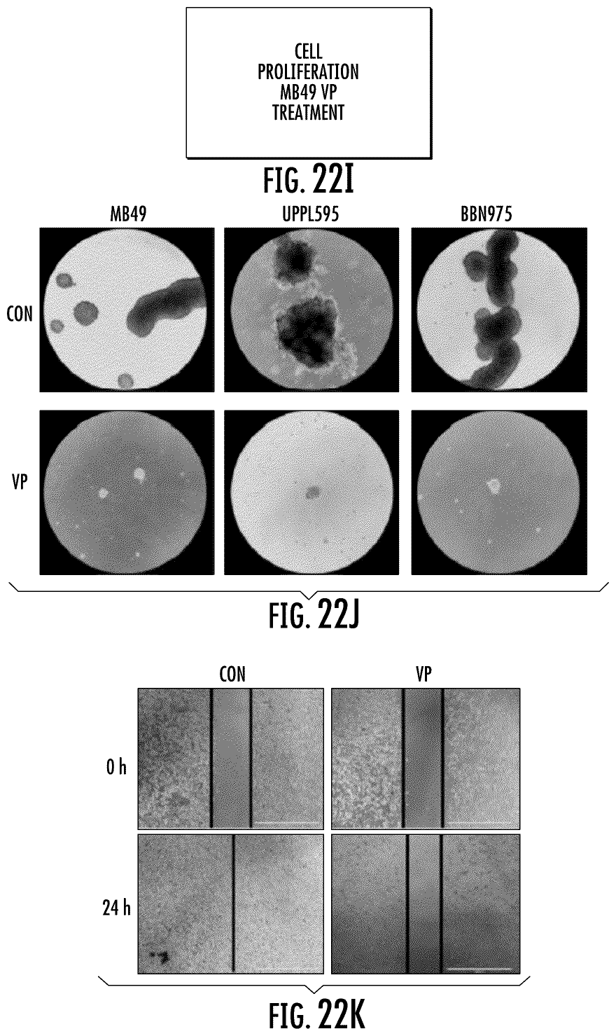

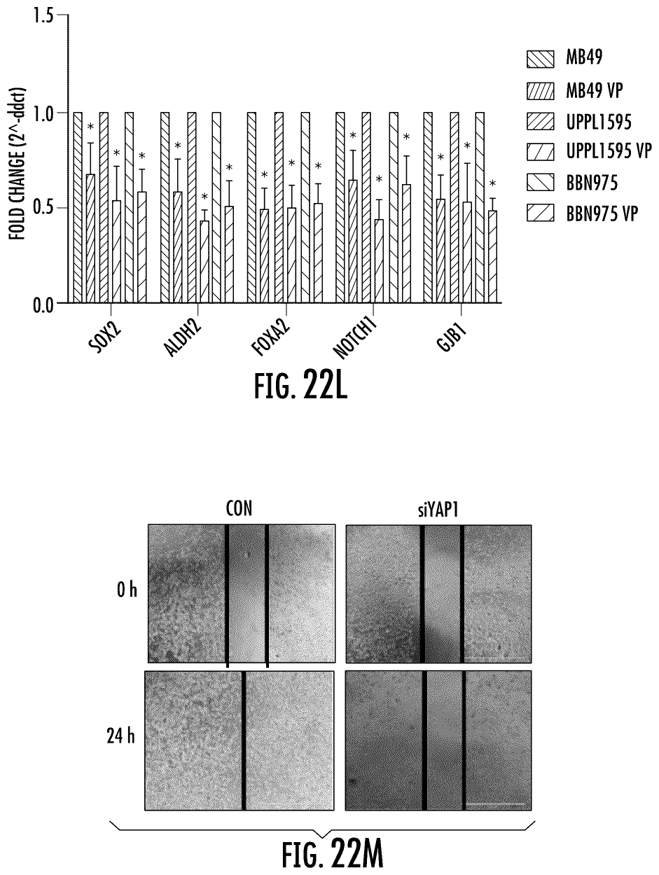

YAP1 is a potential driver candidate for poor overall survival of UCB patients and cellular malignant stemness. Analysis of TCGA (The Cancer Genome Atlas) database suggested significantly poor overall and disease specific survival of UCB patients with high YAP1 expression (FIG. 22A-22B). Moreover, a positive correlation of YAP1 expression with potentially known oncogenes (such as TMEM123, DCUN1D5, BRIC2, KRAS, DYCH2H1, PWP1 and RABEPK) were observed (not shown).

Although in its infancy, there are some reports indicating that YAP1 might play a role in the development of immunosuppressive TME (37). To test this hypothesis in UCB, YAP1 knockdown (KD) clones were prepared using MB49 cell line (FIG. 22C), a mouse derived UCB cells with high level of endogenous YAP1 expression. Initial characterization of these KD clones indicated that, YAP1 KD in the MB49 cells significantly attenuated the cell proliferation rate (FIG. 22D). Similar to the present inventors' previous studies in human UCB and lung adenocarcinoma cell lines, (7, 38), MB 49 YAP1 KD clones formed smaller and less spheres compared to control cells (FIG. 22E). Furthermore, expression of several cancer stem cell (CSC) markers such as SOX2, GJB1, FOXA2, Notch1, and ALDH2 decreased due to YAP1 KD in MB49 cells that suggest malignant stemness of cell population decreased due to down regulation of YAP1 signaling (FIG. 22F). Like MB49, UPPL595 and BBN975 cells also expressed high endogenous YAP1. To confirm the phenotypic changes observed due to genetic YAP1 inhibition in MB49 cells, the present inventors pharmacologically inhibited YAP1 in all the three cell lines (MB 49, UPPL595 and BBN975) by treating with a potent and specific YAP1 inhibitor [verteporfin, (VP, 1 μM](FIG. 22G) and cell proliferation, sphere formation and RT-qPCR analysis of candidate CSC markers were performed. As expected, consistent with genetic YAP1 attenuation in MB49 cells, cell proliferation, sphere formation and RT-qPCR analysis of CSC markers indicate that VP can inhibit the development of cancer stemness in the mouse UCB cell lines (FIG. 22I-22K). Additionally, genetic and pharmacologic inhibition of YAP1 activity showed significant difference in wound closure (FIG. 22L-22M). Overall, these findings indicate the oncogenic potential of YAP1 in UCB.

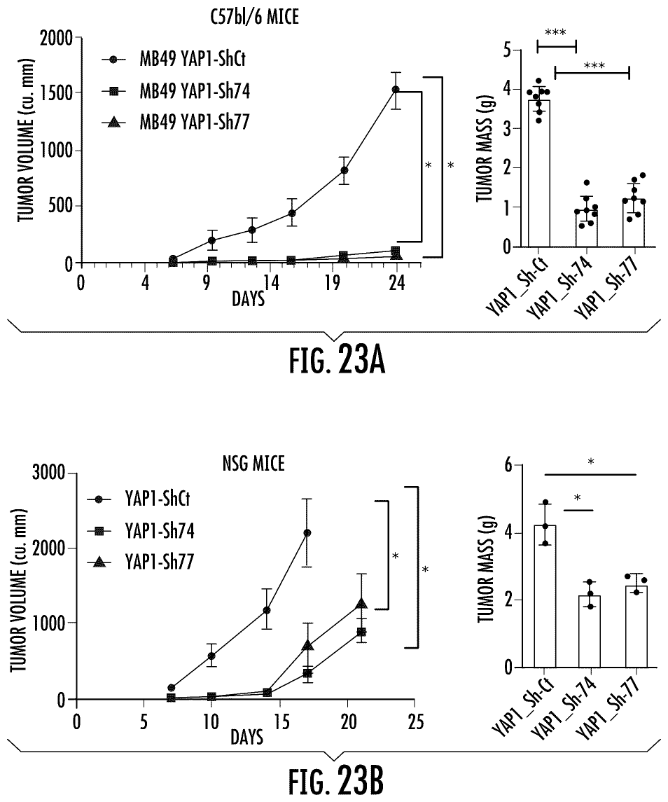

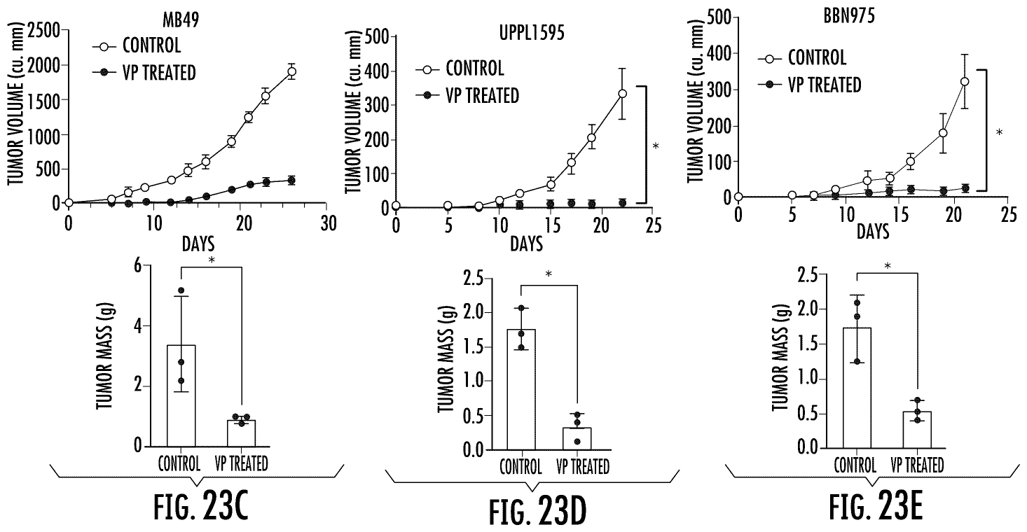

Attenuation of YAP1 inhibits tumor growth in vivo. After confirming the oncogenic potential of YAP1 in vitro, the oncogenic effect of YAP1 was evaluated in vivo in immune competent as well as in NSG mice (immunocompromised). Cell derived xenografts (CDX) were developed in immune competent mice (C57BL6) using different YAP1 KD (clones Sh74 and Sh77) and Sh-control MB49 cells. Both the KD clones showed significant tumor growth regression compared to the Sh-control clones (FIG. 23A left). As expected, tumor mass also decreased significantly in the KD clones compared to the control clones (FIG. 23A, right). To explore any possible role of the endogenous immune system in tumor development in context with YAP1 expression, a CDX model were developed with same cell line (MB49) in an immune compromised NSG mice. Interestingly, for the first two weeks after cell implantation, the tumor growth of KD clones were significantly attenuated compared to the control cells (FIG. 23B). However, beyond this time point, although the overall tumor volume significantly less in the KD cells compared to the control cells, the volume of the YAP1 KD tumors rapidly increased when compared to tumor growth in immune competent mice (FIG. 23A). The tumor mass values also showed a significant difference between the KD and control tumors but the magnitude of differences is broader in immune competent mice compared to NSG mice (FIG. 23A right, 2B right).

To test the pharmacological inhibition of YAP1 on in vivo tumor growth in NSG and immune competent mice, CDXs were developed using three different mouse derived cancer cell lines (MB49, UPPL596 and BBN 975). Verteporfin was administered interperitoneally at a dose of 50 mg/kg body weight at every other day. For all the three cell lines, a significant regression in tumor growth was observed in response to VP treatment in immune competent mice (FIG. 23C-23E; top). Tumor mass values also indicate similar trend (FIG. 23C-23E; bottom). The pharmacodynamic analysis also showed decrease YAP1 expression in the tumor tissue taken from the VP treated animals (FIG. 23F). To validate the attenuation of YAP1, RT-qPCR analysis was carried out on CCN1 and CCN2 expression (YAP1 downstream genes) and the expression for both the genes found to be downregulated upon VP exposure (FIG. 23G).

In summary, comparing tumor growth in NSG and immune competent mice, the present inventors' results indicate that genetic and pharmacologic attenuation of YAP1 significantly regress the tumor development in immune competent and NSG mice. However, in NSG mice, YAP1 inhibition in not standing out to be an effective way to inhibit tumor growth over the time. These interesting observations prompted us to test a hypothesis that endogenous immune system might have a significant role in tumor development in context with YAP1 expression in the tumor cells. In the subsequent experiments, the goal is to dissect out a possible mechanism behind the development of immunologically hot TME due to YAP1 inhibition in UCB.

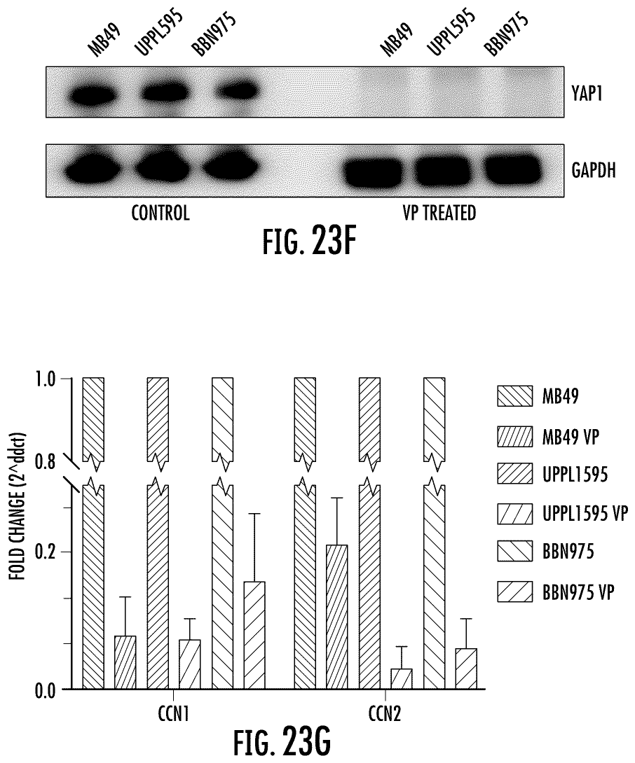

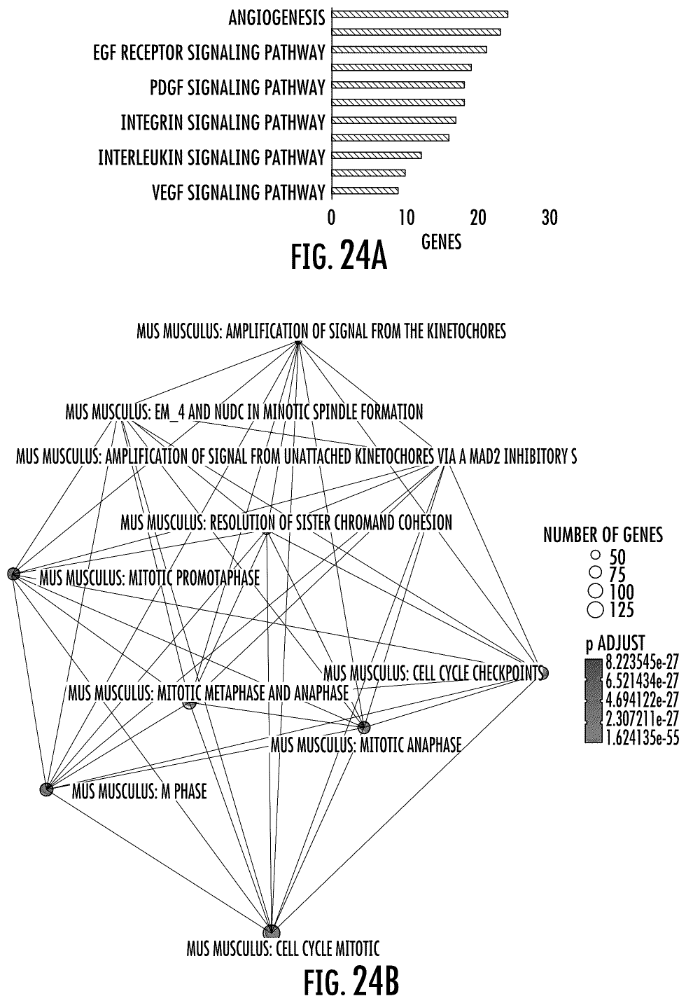

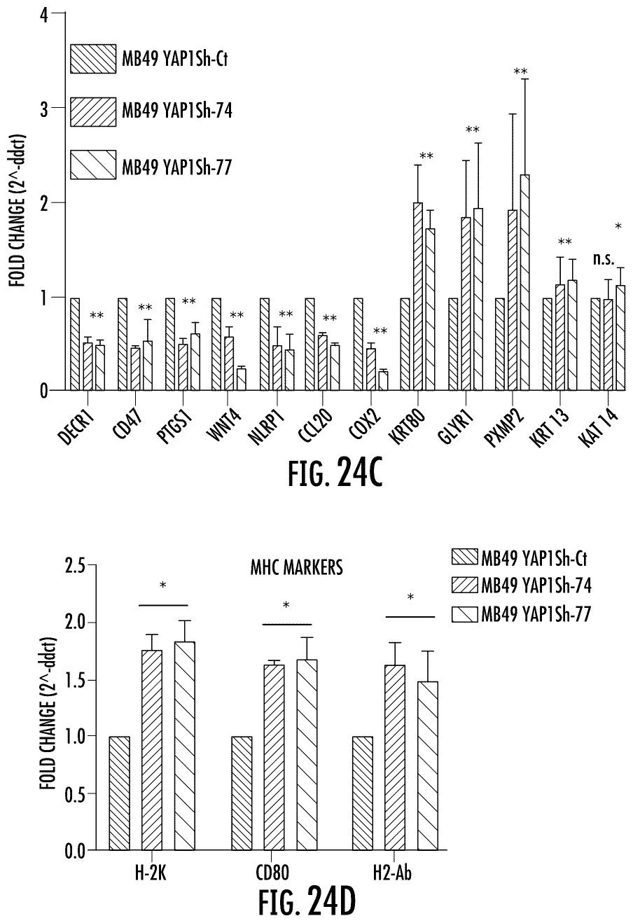

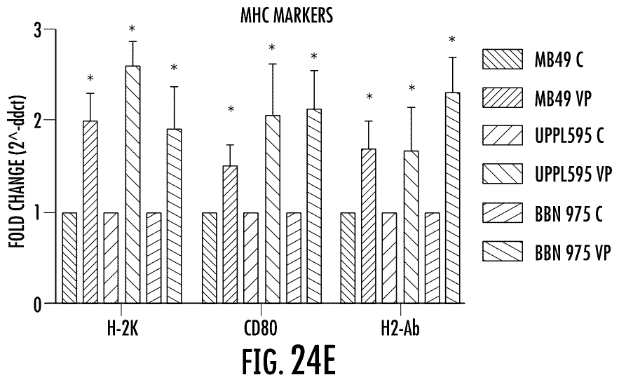

YAP1 drives immune suppression in UCB. To investigate the downstream targets in YAP1 signaling induced tumorigenesis, the present inventors did RNA sequencing of mice and human cell lines (MB49 YAP1 KD and BFTC 905 YAP1KD clones of mice and human respectively with appropriate controls). Analysis of RNA-seq data of both mice and human cell lines with different level of YAP1 expression indicated a possible YAP1 driven enrichment of signaling pathways associated with tumorigenesis and tumor immune evasion. For example, analysis of the RNA seq data of MB49 cells indicated that YAP1 KD lead to downregulation of several oncogenic and immune evasion associated pathways including angiogenesis, EGF receptor signaling, PDGF signaling, integrin signaling and VEGF signaling (FIG. 24A). The computational analysis also indicates that the deregulation of interleukin signaling pathway in the YAP1 expressing cell line correlated with upregulation of the cell cycle regulatory proteins in the YAP1 KD MB49 clone (FIG. 24B). Similarly, analysis of the RNA seq data from BFTC905 cells indicated a similar trend of deregulated cytokine and chemokine signaling (not shown). Analysis of these data using another computational tool (STRING) confirms that YAP1 promotes the expression of immunosuppressive cytokine and chemokine signaling. To understand the TIME in human samples in context with YAP1 expression, the present inventors analyzed the UCB-TCGA database and the present inventors' analysis revealed that CXCL10 (an immunosuppressive chemokine) is one of the top overexpressed genes in YAP1 upregulated UCB samples (not shown). The present inventors performed RT-qPCR of candidate genes to validate the in silico data and found that genes that promote tumorigenesis and immune evasion are significantly downregulated in YAP1 KD clones (FIG. 24C). Furthermore, by q-RT-PCR analysis the present inventors found that YAP1 attenuation (by genetic and pharmacological means) upregulates the expression of MHC markers such as H-2K and CD80 (FIG. 24D-24E). In summary, the analysis of RNA-seq data using mice and human cell lines with different level of YAP1 expression and primary TCGA-UCB samples allowed us to hypothesize that YAP1 might has a significant role in tumor immune evasion.

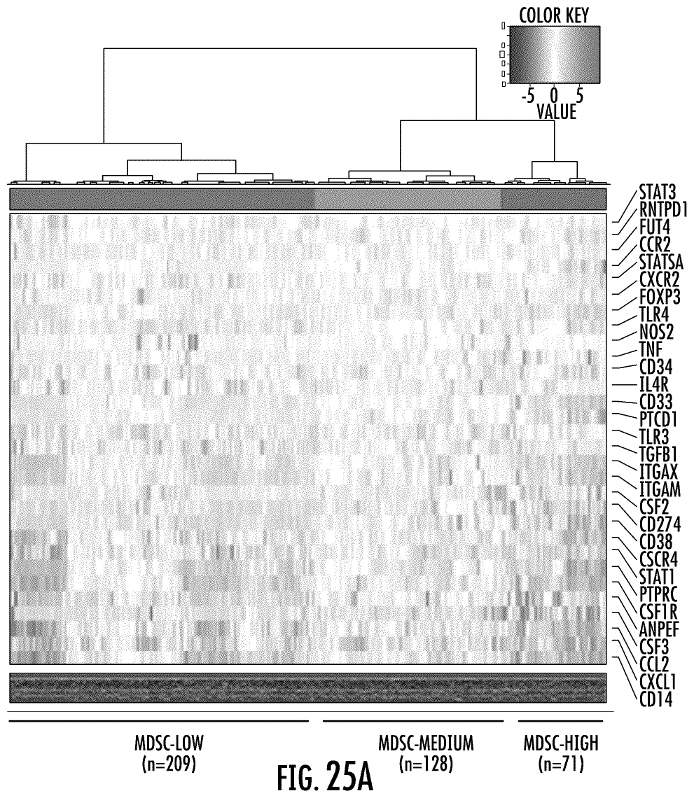

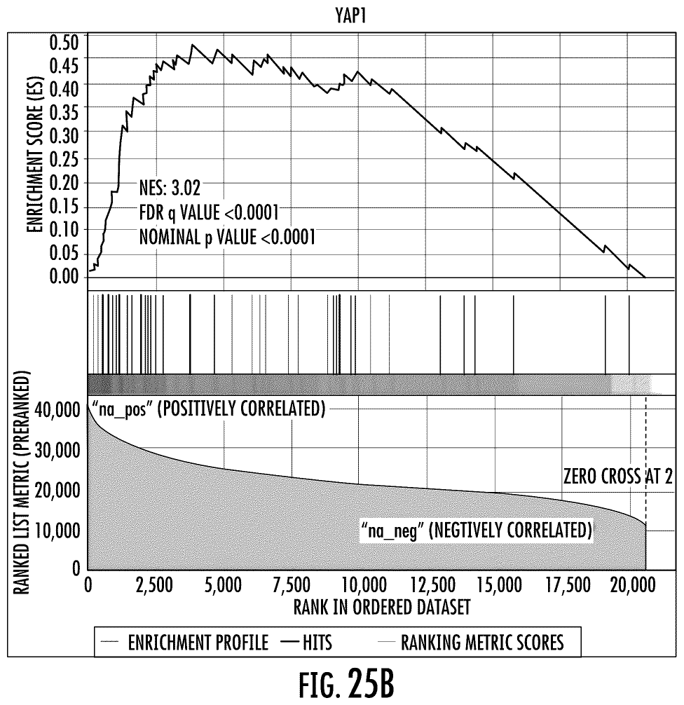

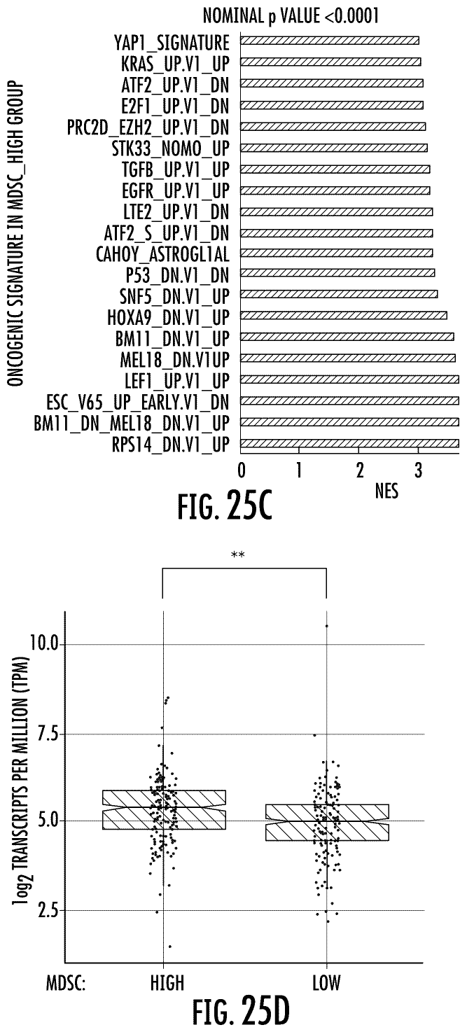

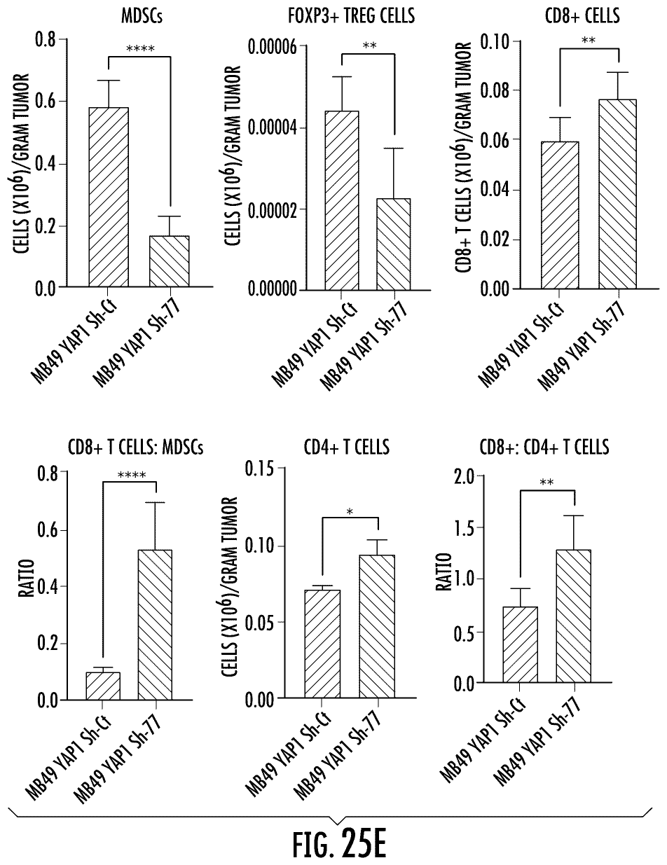

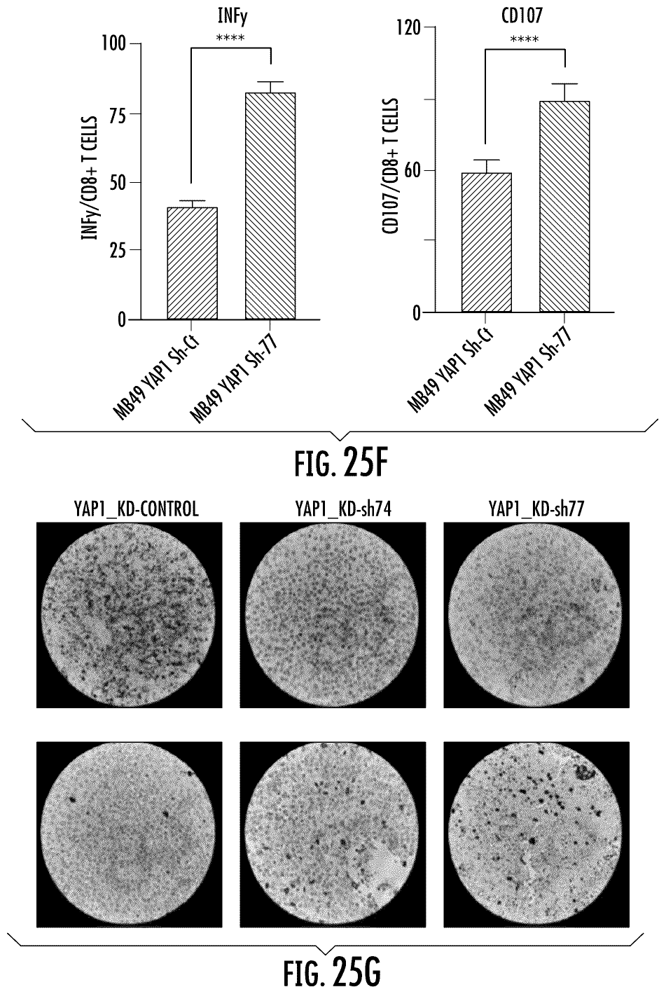

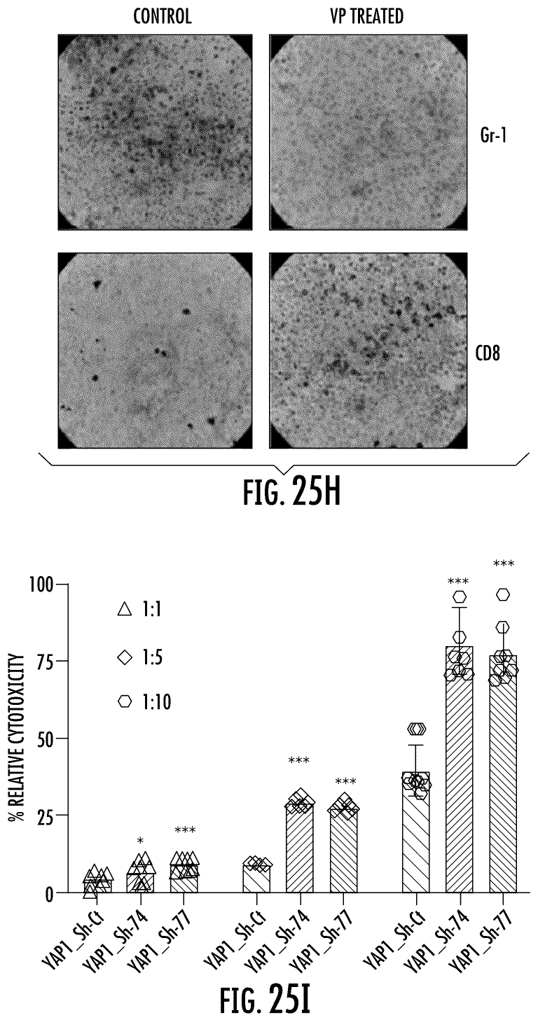

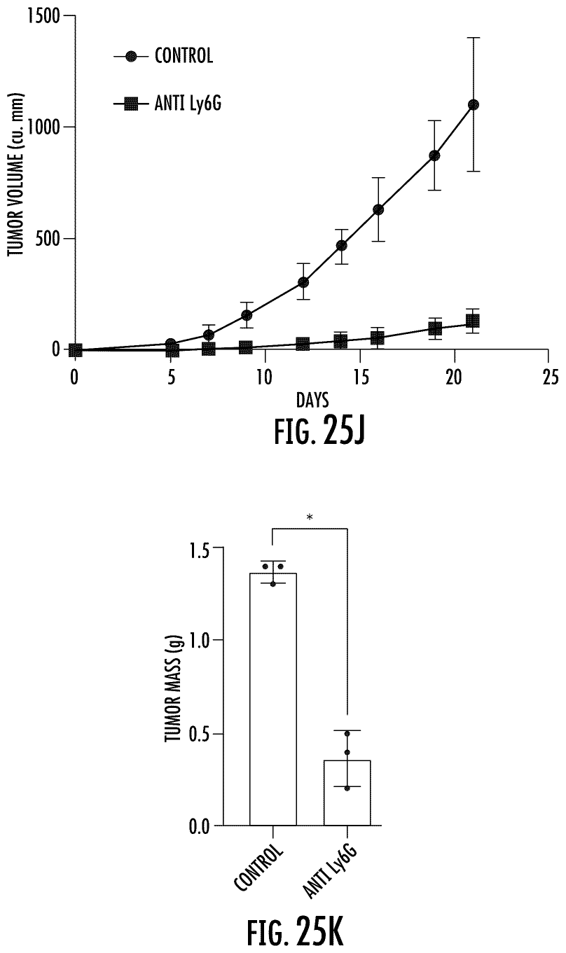

YAP1 induces the immune suppressive TME. Analysis of the TCGA database indicates that high YAP1 expression associated with enriched signaling cascade of MDSC in UCB (FIG. 25A). Furthermore, the gene set enrichment analyses (GSEA) revealed that high presence of MDSCs in the tumor tissue results in upregulation of different oncogenic pathways compared to low MDSC frequency, and YAP1 is one of the topmost upregulated gene signatures among high MDSC UCB samples (FIG. 25B). FIG. 22c showed significantly high normalized enrichment score (NES) of YAP1 in high MDSC infiltrated samples compared to low MDSC infiltrated samples YAP1 expression itself also found to be higher in high MDSC infiltrated UCB samples (FIG. 25D). The present inventors' RNA-seq data (FIG. 24A) and two publicly available primary UCB data base (TCGA and IMVIGOR 210) analysis indicate that YAP1 is closely associated with several immune regulating pathways. Therefore, the present inventors speculate that YAP1 might have a major role in the regulation of TIME. To further understand the role of YAP1 in immune regulation of TME and for experimental validation, the present inventors developed cell derived xenograft models in C56/BL6 mice using MB49 YAP1 sh-control and MB49 YAP1 KD clone (YAP1 Sh-77). As expected, the number of MDSCs and FOXP3+ T cells were significantly less in YAP1 KD tumors compared to sh-control tumors (FIG. 25E). Additionally, an increased level of CD8+T and CD4+ T cells were observed in the YAP1 KD tumors (FIG. 25E). The ratio of CD8+ T cells and MDSCs was higher in YAP1 KD tumors (FIG. 25E). Similarly CD8+ T cells and CD4+ T cells ratio was higher in YAP1 KD tumors. Moreover, expression analysis of activation markers CD107 and IFNγ indicate higher T cell activation in the YAP1 attenuated tumor tissues (FIG. 25F) compared to controls. For further validation, the present inventors performed immunohistochemical staining (IHC) of Gr-1 and CD8 using the tumor tissue derived from YAP1 expressed and KD MB49 cells and the present inventors' findings were in consistent with FACS analysis (FIG. 25E) that is decreased numbers of MDSCs (Gr-1) and increased numbers of CD8+ T cells in YAP1 KD tumors (FIG. 25G). Treatment of mice with YAP1 inhibitor in YAP1 expressed MB49 xenografted tumors also showed the similar pattern of MDSC and CD8 cells infiltration (FIG. 25H). These findings clearly indicate that YAP1 attenuation increases the infiltration of the CD8+ cells and decreases the infiltration of MDSC in the TME. Simultaneously, YAP1 attenuation is associated with increased CD8-T cells cytotoxicity (FIG. 25I). To explore whether MDSCs are one of the critical factors driven by YAP1 in UCB tumorigenesis, a separate CDX model was developed with wild type MB49 cells. The animals were interperitoneally administered with anti-GR-1 antibody every day for 3 weeks. Tumor progression rate and tumor mass indicate that anti Gr-1 antibody significantly downregulated tumor growth compared to the animals administered with IgG control (FIG. 25J-25K). Overall, the present inventors' findings indicate that YAP1 is one of the critical factors in inducing immunosuppressive TME by increased infiltration of MDSCs and lowering the activity and infiltration of cytotoxic T cells.

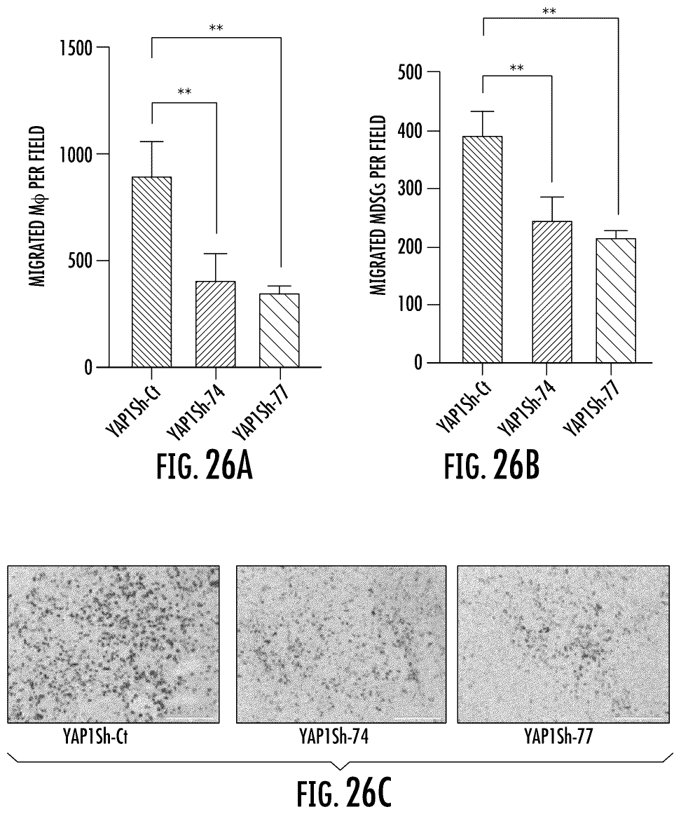

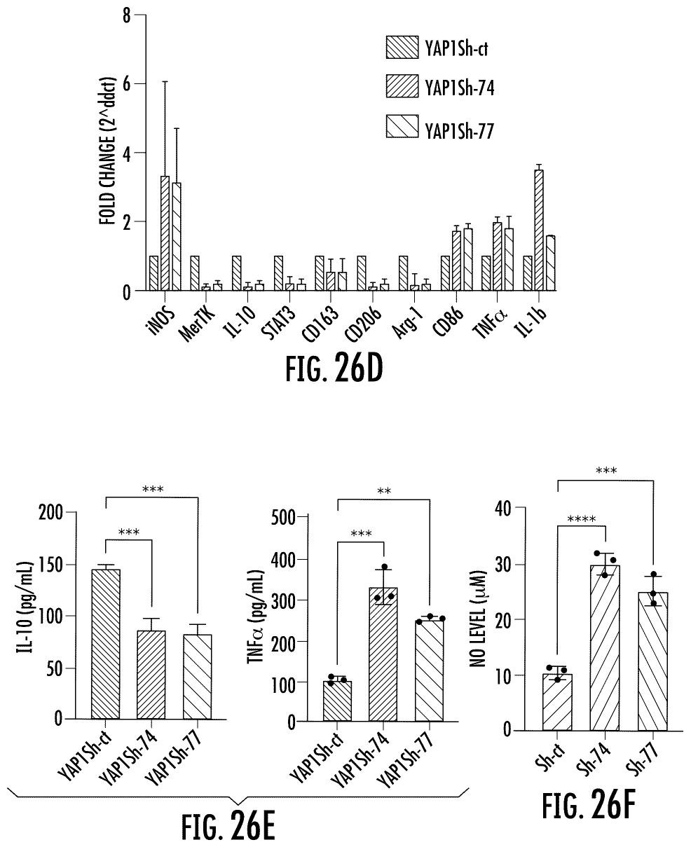

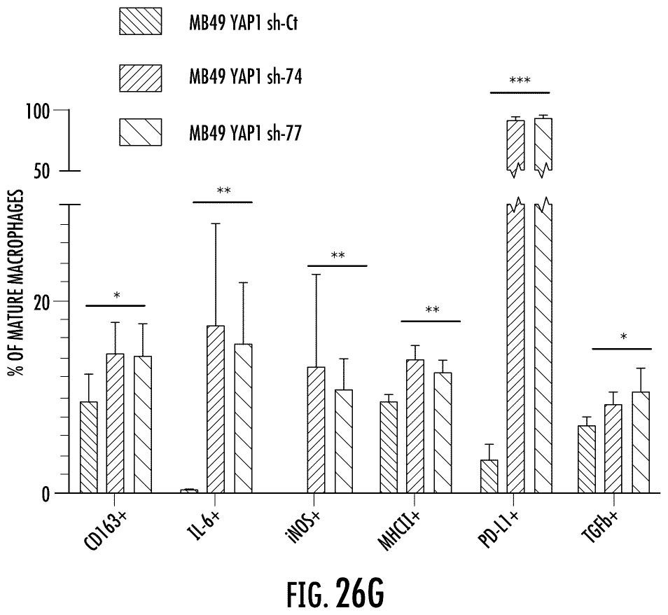

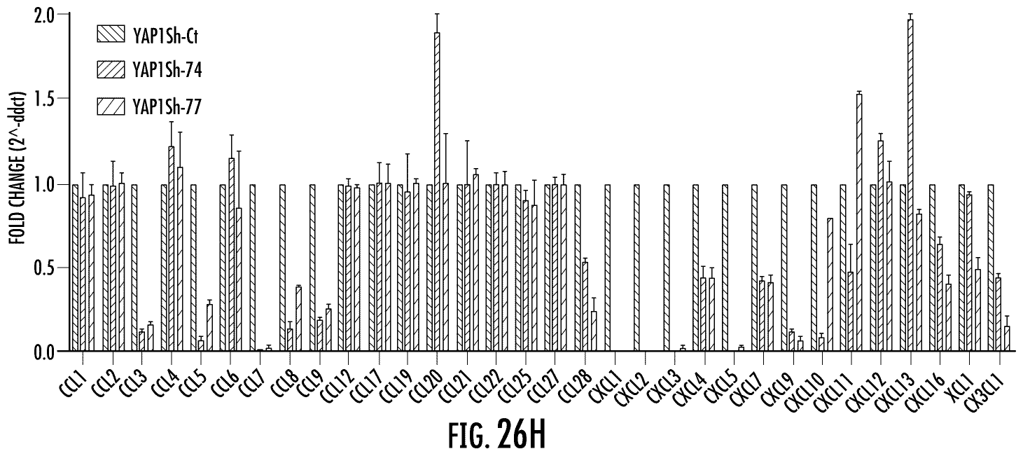

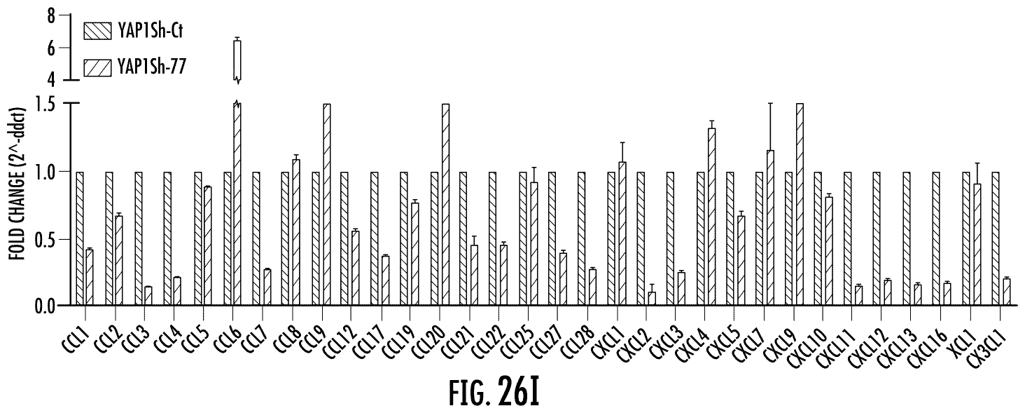

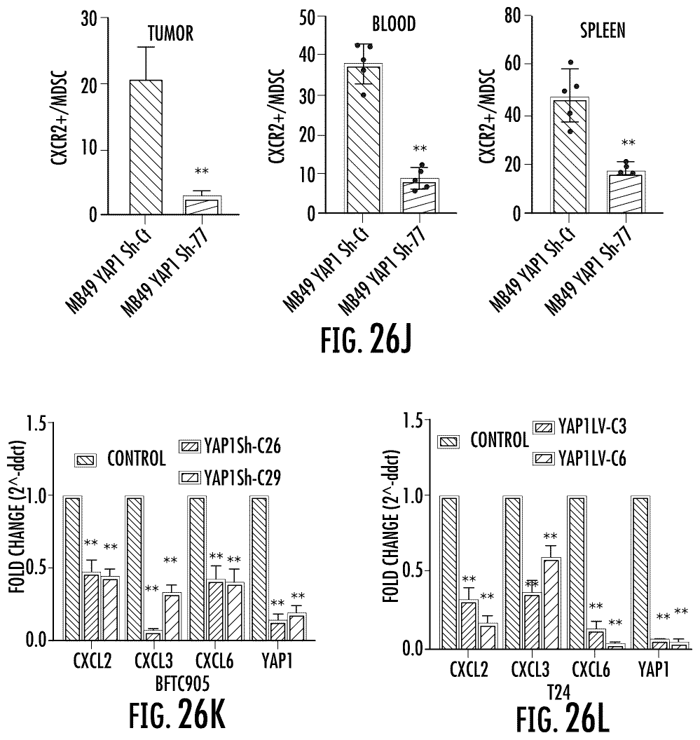

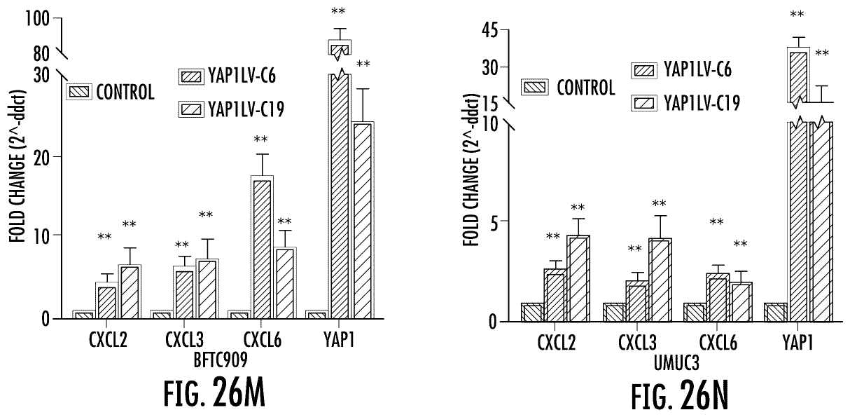

YAP1 influences the migration of MDSC and migration and polarization of macrophages. TME drives the numerous phenotypic changes including metastasis, angiogenesis, cancer stemness and immune evasion associated with tumor initiation and progression (39, 40). To get further insight of TME in YAP1 context, MDSCs were isolated from tumor mass and primary macrophages were isolated from the intraperitoneal cavity of same tumor bearing mice developed from MB49 YAP1 KD and sh-control clones. To understand the influence of YAP1 on infiltration of MDSC and macrophages, the present inventors performed migration assay using isolated MDSCs and macrophages at the upper chamber and conditioned media (CM) collected from YAP1 KD and sh-control clones at the bottom chamber. The present inventors' results indicate that conditioned media from sh-control cells attracts more macrophages (FIG. 26A) and MDSCs (FIG. 26B) in the bottom chamber compared to YAP1 KD clones. IHC micrographs (F4/80) also indicate less infiltration of macrophages in TME in YAP1 attenuated condition compared to control (FIG. 26C).

The intrinsic functional plasticity of macrophages leads the investigators to develop strategies to reprogram TAMs from M2-like immunosuppressive and tumor-promoting cells into M1-like macrophages with immunostimulatory, antitumor phagocytic and cytotoxic activities. To determine any association between the influence of YAP1 expression in macrophage polarization, the present inventors performed RT-qPCR analysis of selected macrophage polarization markers. The present inventors found decreased expression of CD206, CD163, MerTK, 11-10, Arg-1 and STAT3 (M2 phenotype markers) in macrophages cultured in the CM of YAP1 KD clones compared to CM of sh-control MB49 clones (FIG. 26D). To solidify RT-qPCR findings, the present inventors performed ELISA for two key M1/M2 factors (I1-10 and TNF-α) using CM of YAP1 KD and sh-control clones and the findings are consistent with RT-qPCR data (FIG. 26E). The present inventors also performed Griess assay for nitric oxide (NO). As expected, NO was increased in macrophages cultured with the CM from YAP1 KD MB49 cells (FIG. 26F). FACS analysis was also performed with these co-cultured macrophages, and it revealed that CM from YAP1 expressing MB-49 cells is significantly polarizing the macrophages into M2 type (FIG. 26G). In summary, the present inventors' findings indicate that TME with YAP1 attenuation might be able to polarize the macrophages into M1 phenotype. These M1 macrophages can be beneficial in regressing tumor progression and induce immunologically “hot” TME.

Genetic knockdown of YAP1 leads to deregulation of immune associated cytokines/chemokines. To further understand the mechanism of YAP1 associated immune microenvironment, the present inventors analyzed a panel of 32 cancer associated cytokines/chemokines on a RT-qPCR array in YAP1KD and sh-control MB49 cells and cell derived xenografts. The array data revealed that YAP1 expressing MB49 cells (FIG. 26H) and xenograft (FIG. 26I) expressed notably increased CXCR2 associated ligands, such as CXCL2, CXCL3 and CXCL5 (FIG. 26H-26I). The present inventors further analyzed the expression of CXCR2 in the primary tumors as well as in the blood and spleen of the tumor bearing animals by FACS. The present inventors' findings revealed that tumors from YAP1 expressing cells (sh-control), expressed increase level of CXCR2 compared to YAP1 KD clones (FIG. 26J).

To determine YAP1 associated regulation of these chemokines in human, the present inventors first performed RT-qPCR analysis of CXCR2 associated ligands (CXCL2, CXCL3 and CXCL6) in human cancer cells (T24, BFTC905, BFTC909 and UMUC3) with YAP1 modulation. Consistent with the present inventors' mouse data, these 3 cytokines were downregulated in YAP1KD human cells (BFTC 905 and T24) while upregulation was observed in YAP1 overexpressed human cells (BFTC909 and UMCU3) (FIG. 26I-26L). The present inventors further analyzed the expression of CXCL2, CXCL3, CXCL6 and YAP1 in a primary UCB cohort (n=30) using RT-qPCR and found significant correlations of YAP1 expression with all these cytokines (not shown). External validation using TCGA UCB cohort generated similar conclusion (not shown). Collectively, the present inventors' results indicate that YAP1 expression led to expression of various cancer promoting chemokines/cytokines and these in turn lead to induction of immunosuppressive TME.

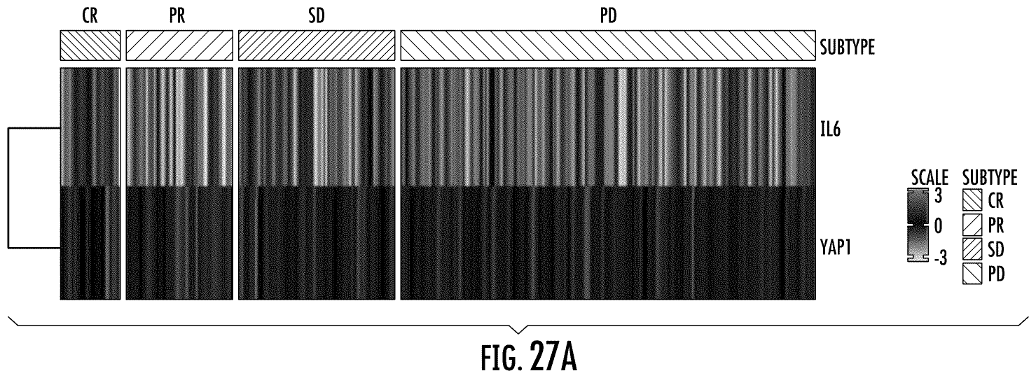

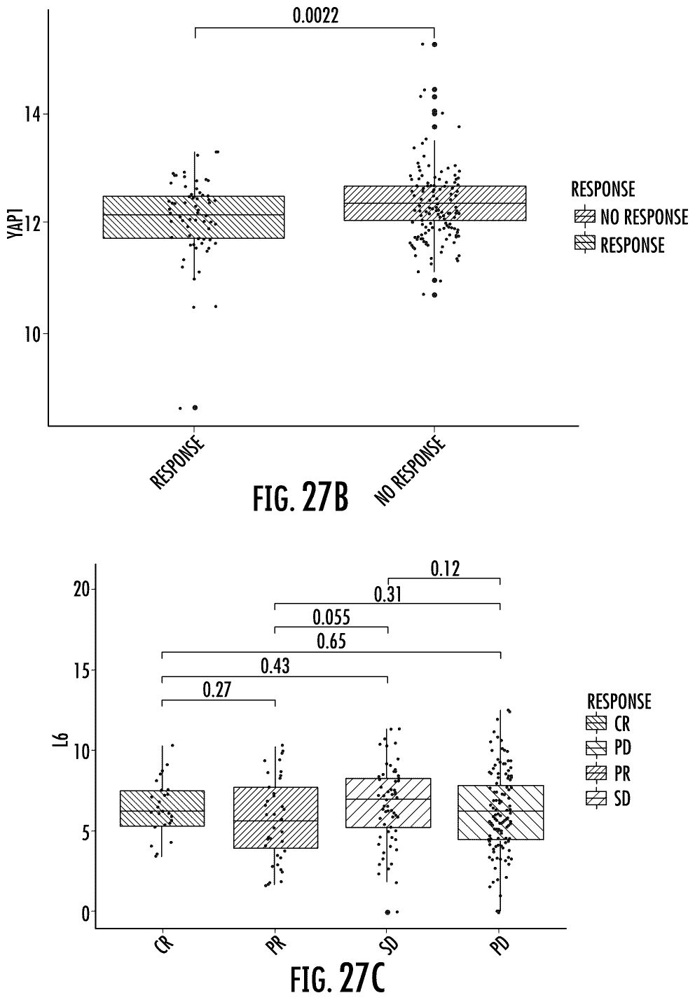

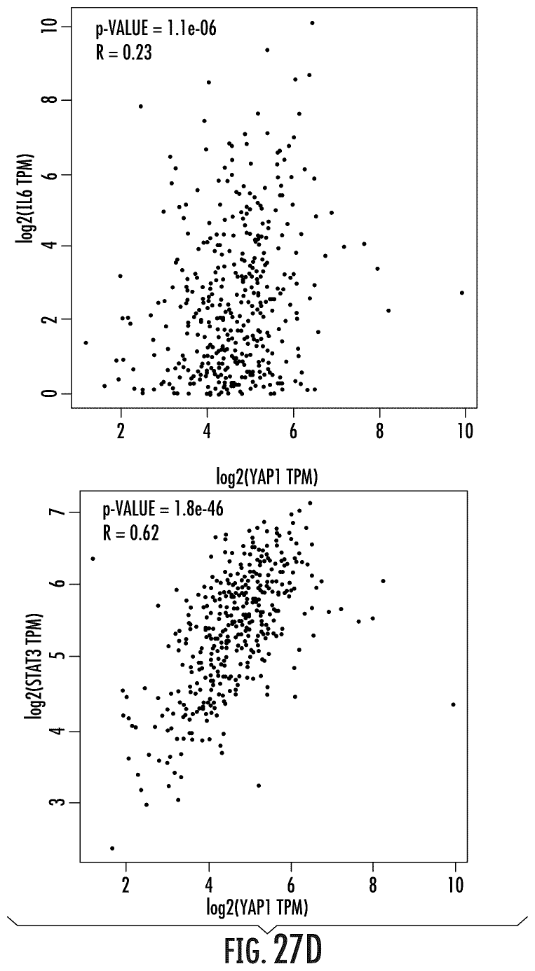

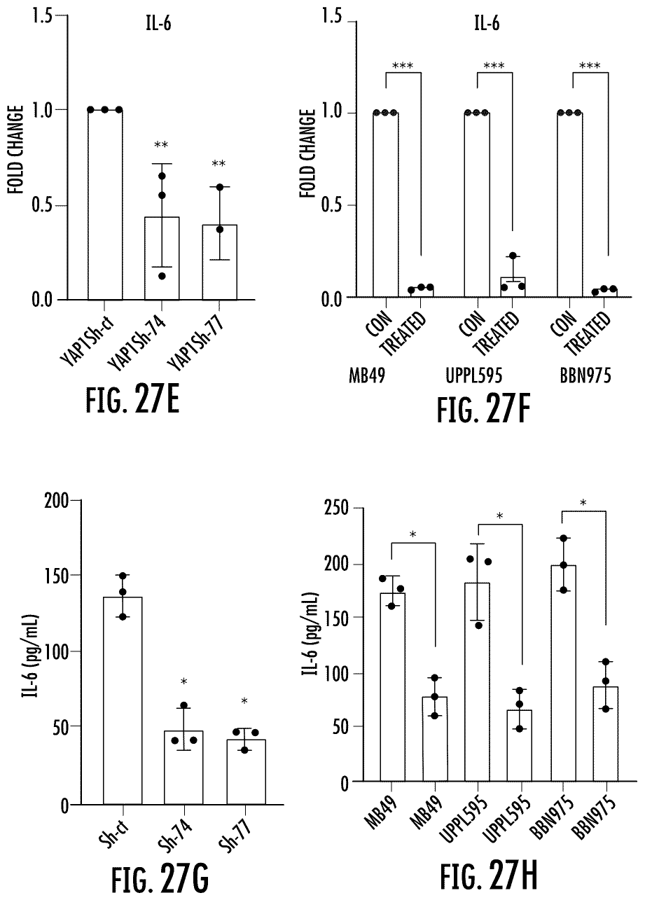

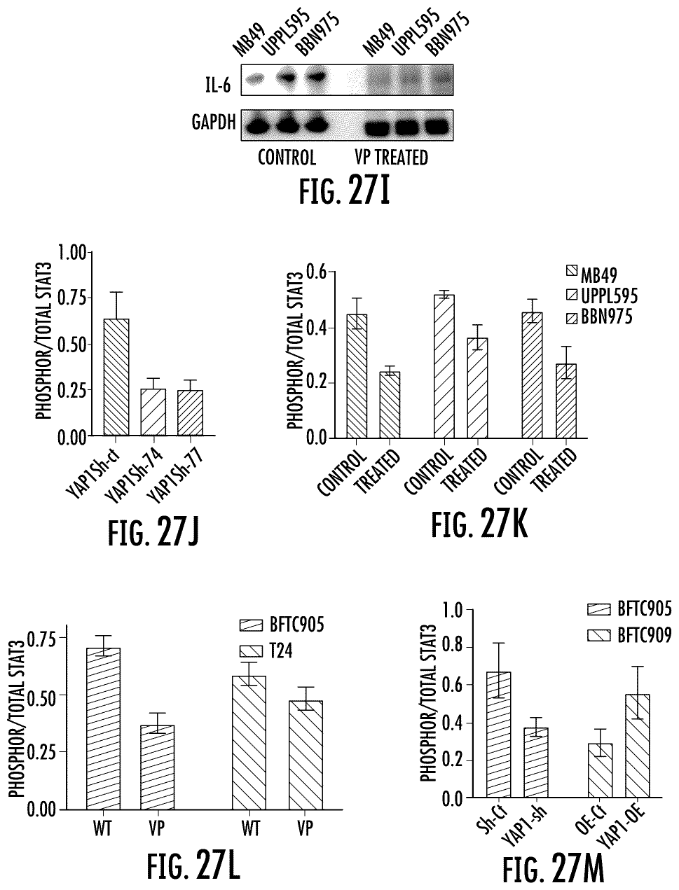

YAP1 activates IL-6/STAT3 pathway during UCB progression. Using lung adenocarcinoma (LUAD) cell lines, the present inventors previously showed that YAP1 bind to the promoter region of IL-6 and induces its transcription that result in upregulation of the phosphorylation of STAT3 (active form) (38) This observation led us to investigate the IL-6 expression in UCB patient samples and cell lines in context with YAP1 expression. Analysis of the publicly available IMvigor210 database revealed that YAP1 expression is significantly upregulated in immunotherapy non-responsive patients (FIG. 27A-27B). IL-6 expression also showed differential pattern based on therapeutic outcome, it is found to be highest in partial response group (PR) ad highest in stable disease group (SD) (FIG. 27C). Furthermore, the present inventors' analysis also showed a trend of decrease overall survival of these patients who expressed both YAP1 and IL-6 (p=0.13). Additionally, analysis of UCB-TCGA database showed that both IL-6 and STAT3 expression is positively correlated with the expression of YAP1 (FIG. 27D-27E).

The above in silico analysis prompted us to explore the correlation of YAP1 with IL-6/STAT3 signaling in UCB. To this end, using several in vitro and in vivo models, the present inventors analyzed IL-6 at transcript and protein level after genetic and pharmacologic attenuation of YAP1. As expected, pharmacologic and genetic attenuation of YAP1 led to downregulation of IL-6 at RNA and protein level (FIG. 27E-27I). As STAT3 is a target of IL-6 (11, 41), the present inventors validated the phosphorylation status of STAT3 in UCB cell lines and xenografted tissues. ELISA with intracellular protein showed that STAT3 phosphorylation is positively correlated with the expression of YAP1 in MB49 cell lines (FIG. 27J) and VP treated mice bearing UCB cell lines derived xenografts developed in immune competent mice (FIG. 27K). Similar data were also observed after pharmacologic inhibition of YAP1 in human UCB cell lines (FIG. 27L). Genetic inhibition of YAP1 in human UCB cell lines also generated similar results (FIG. 27M).

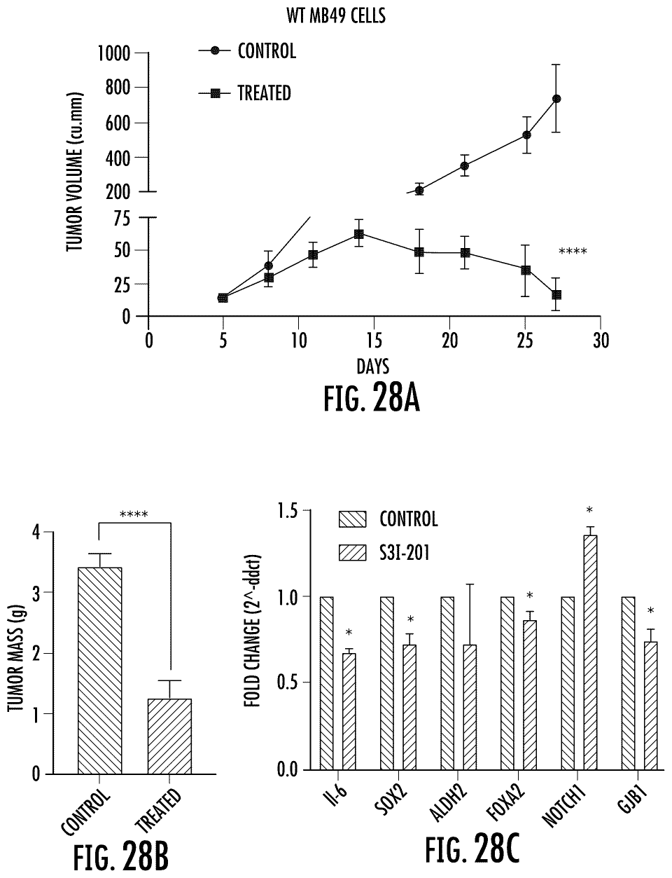

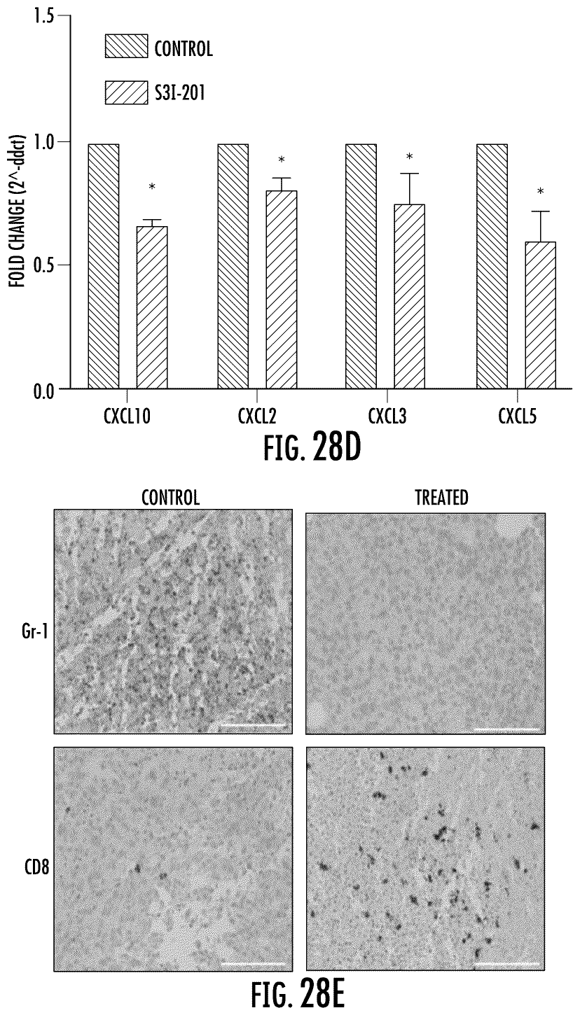

YAP1 induces immunosuppression partially through IL6/STAT3 signaling and STAT3 inhibition mimics the antitumor activity of YAP1 attenuation. The present inventors recently reported that YAP1 positively regulates IL6/STAT3 signaling (38) in LUAD. Different studies suggest that CXCLs are critical players in inducing immunosuppressive TME through the infiltration of MDSCs in tumor site and cytotoxic T cell exhaustion (40, 42, 43). As the present inventors' data suggest that YAP1 has influence on CXCLs expression (FIG. 26), the present inventors explored the YAP1-IL6-STAT3-CXCLs signaling in MB49 derived xenograft in C57BL/6 mice. Treatment of the tumor bearing animals with S3I-20I (STAT3 inhibitor) resulted in significant regression of the tumor growth (FIG. 28A-28B). The qRT-PCR analysis showed significantly decreased expression of CSC associated markers and CXCLs in tumor tissues of STAT-3 inhibitor treated group (FIG. 28C, 28D) which is generally similar to YAP1 attenuation in this cell lines (FIG. 22, FIG. 26). Of note, the IHC analysis reveals that STAT3 inhibition leads to decreased infiltration of MDSCs (GR-1) and significantly more CD8+ T cells in the TME (FIG. 28e) which is also in agreement with YAP1 inhibition (FIG. 25). These data allow us to infer that YAP1 signaling possibly inducing immunosuppression through IL6/STAT3 signaling.

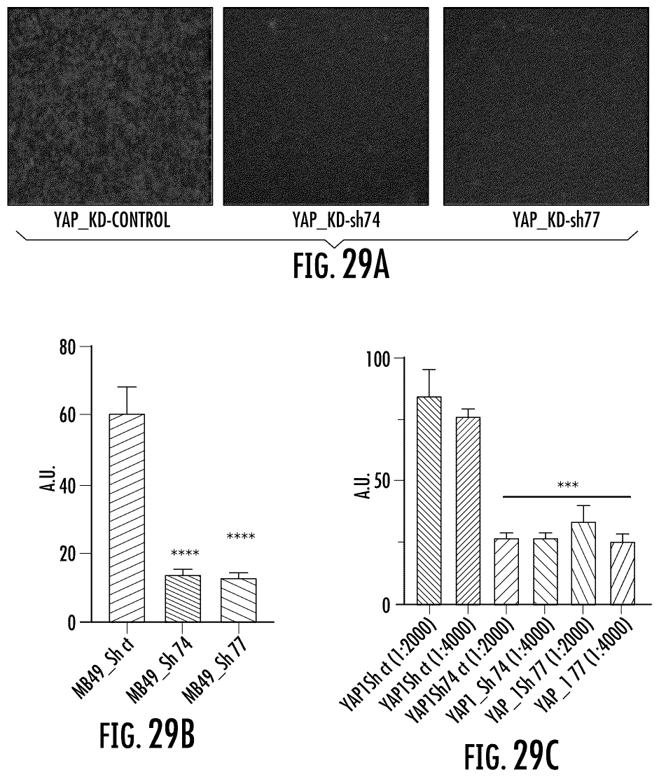

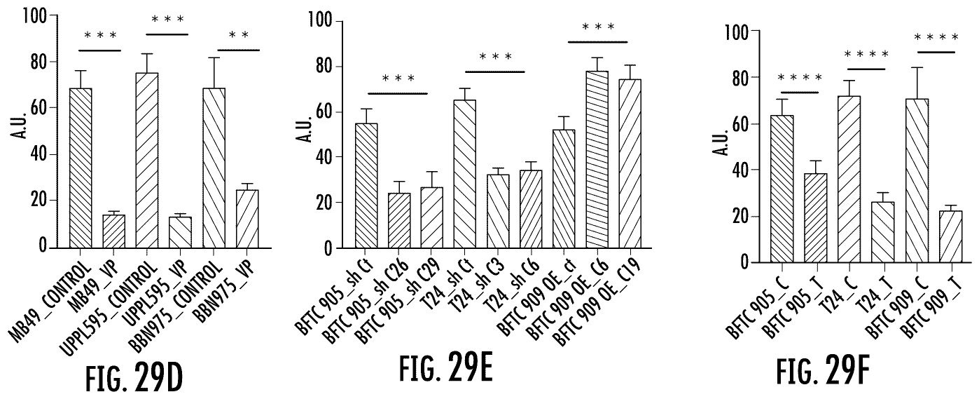

YAP1 influences the accumulation of lipid droplets in cancer cells. Recent studies suggest that metabolic changes (such as accumulation of lipid droplets and deregulation of glycolysis) in cancer cell play a major role in tumor aggressiveness, development of cancer stemness, metastasis, immune evasion and leading to worst prognosis of the disease (44). Therefore, apart from investigating the altered regulation of intracellular signaling cascades, the present inventors also studied cellular metabolism in response to YAP1 modulation. From the RNA-seq data of YAP1 modulated MB49 cells, FABP4 was one of the top upregulated targets in YAP1 expressed cells. It was reported that FABP4 is a critical molecule in accumulation of lipid droplets in the intracellular compartments (45). Therefore, the present inventors validated FABP4 by RT-qPCR from different MB49 YAP1 clones and found that FABP4 expression directly correlated with YAP1 expression (not shown). Pharmacologically, the expression level of FABP4 was significantly downregulated after treatment of mice UCB cell lines (MB49, UPPL595 and BBN975) with YAP1 inhibitor VP, (now shown). Fluorescent microscopy and fluorometric quantification of lipid droplet in YAP1 modulated MB49 cells revealed that YAP1 expression facilitates the accumulation of lipid droplets in the cancer cells (FIG. 29A-29B). It was also observed that when the culture media was supplemented with oleic acid (commonly used as an inducer for lipid droplet formation), there is an increase accumulation of lipid droplets in the YAP1 expressing cells compared to the KD cells (FIG. 29C). Similar results were observed in the VP treated UCB cell lines (MB49, UPPL595, BBN975) derived xenografts (FIG. 29D). The present inventors also quantified lipid droplets in YAP1 KD and VP treated human UCB cell lines (T24, BFTC905 and BFTC909) and found that YAP1 attenuation decreases the accumulation of intracellular lipid droplets (FIG. 29E-29F). As increased amount of L-lactate correlated with more glycolysis and poor prognosis of cancer (46), the present inventors quantified L-lactate in mouse and human UCB cells with YAP1 modulation. As expected, YAP1 attenuation in mouse (FIG. 29G-29H) and human UCB cells (FIG. 29I-29J) showed decreased level of L-lactate.

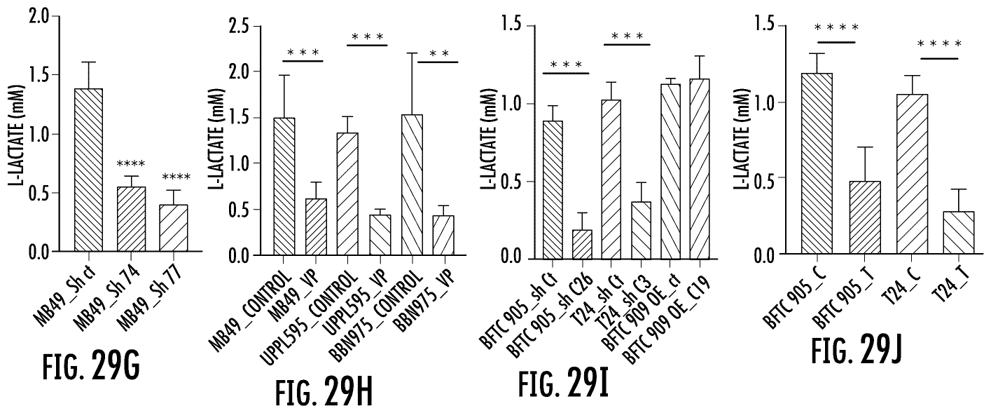

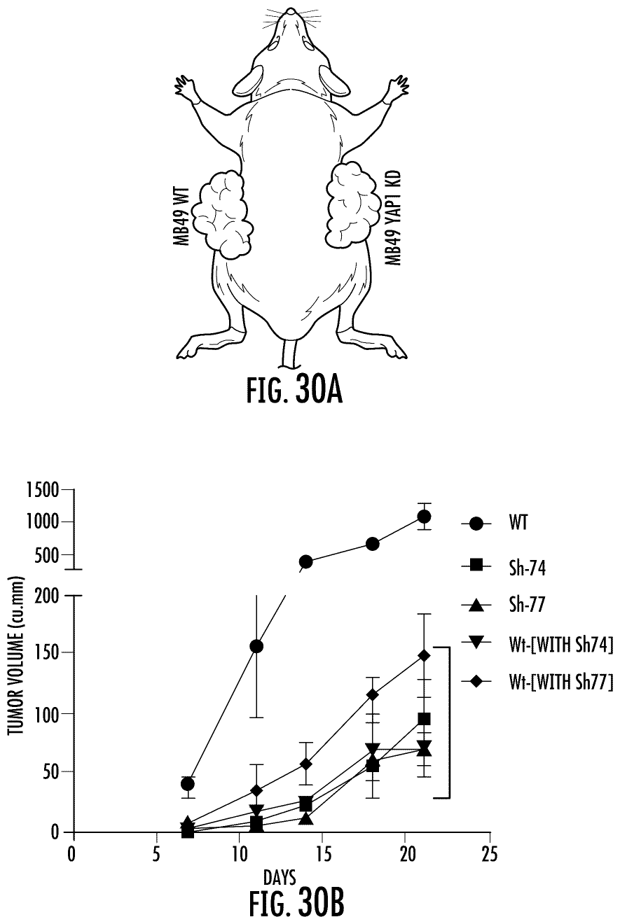

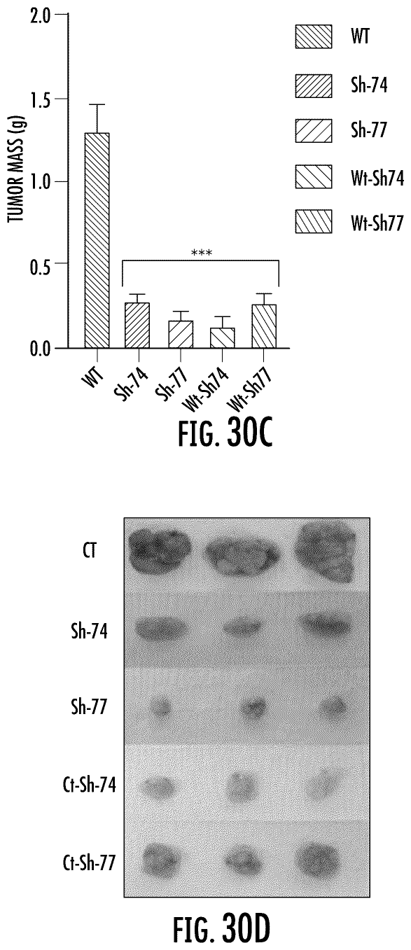

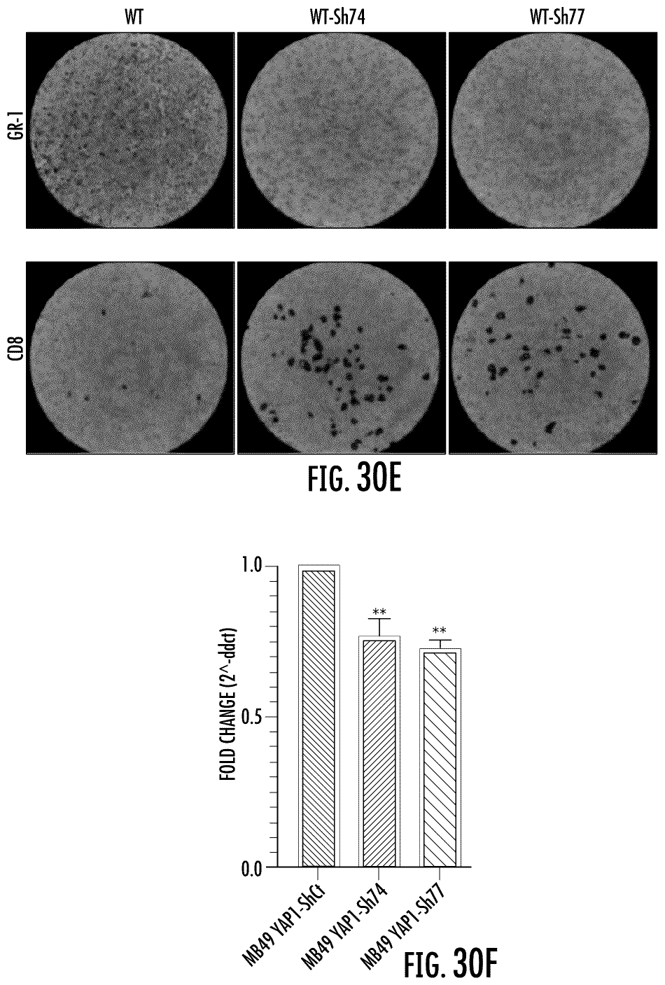

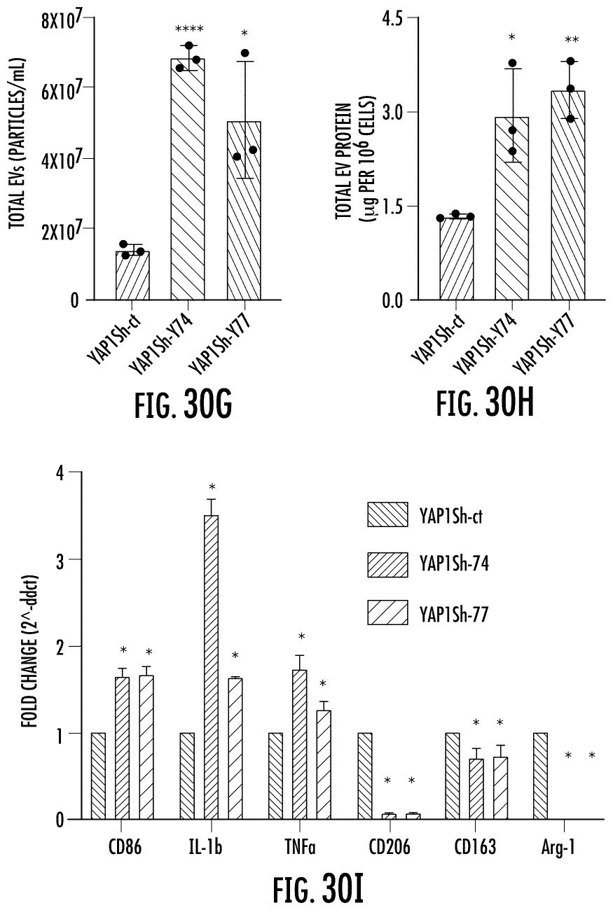

YAP1 deregulation modulate host adaptive immunity by influencing the secretion of EVs in TME. The results described above indicate that YAP1 expression induces immunosuppressive TME and attenuation of YAP1 through genetic or pharmacological approach resulted in enhanced anti-tumor immune response. This observation led us to hypothesize that inhibiting YAP1 will modulate TME and promote the host adaptive immune response. To test this hypothesis, an animal model was developed by injecting YAP1 KD MB49 clones and YAP1 expressing WT MB49 cells into the opposite flank of the same mouse at the same time (FIG. 30A). Interestingly, YAP1 expressing WT-MB49 tumors were significantly less in size in simultaneously YAP1 KD clone and YAP1 expressing WT-MB49 implanted mice when compared to YAP1 expressing WT-MB49 tumors implanted in a separate animal without injecting YAP1 KD MB49 clones (FIG. 30B). The tumor growth curve, tumor mass and the tumor pics clearly indicate that when WT MB49 cells were injected in the opposite flank of the YAP1 KD clones (Sh74 and Sh77) site, the growth rate and tumor development was significantly attenuated (FIG. 30B-30D). IHC analyses revealed decrease infiltration of the MDSCs in tumor of YAP1 KD and WTMB49 grown in same mice compared to WTMB49 tumors grown in separate mice (FIG. 30E). Interestingly, CD8+ T cells were noticeably increased in WTMB49 tumors of grown in the same mice with YAP1 KD tumors compared to WTMB49 tumors grown in separate mice (FIG. 30E). Notably, IL-6, a critical factor in tumorigenesis and immune evasion found to have less expression in the WTMB49 tumors grown in the opposite flank with YAP1 KD tumors compared to WT-MB49 tumors grown in a separate mice (FIG. 30F). In summary, these results indicate that YAP1 expression regulate some critical factors that possibly induce immune suppressive TME in in vivo.

Recently numerous studies suggested that certain secretory molecules play an important role in the development of adaptive immunity and extracellular vesicles (EV) are one of the major sources of these secretory molecules (30). To explore global changes of EV due to YAP1 modulation, the present inventors measure EVs from YAP1 KD and control cells. As expected, the EVs from the YAP1 KD cells were higher compared to control cells (FIG. 30G). Protein quantification analysis also revealed that the protein content of the EV isolated from the YAP1 KD clones are significantly higher compared to the control WTMB49 cells (FIG. 30H). To explore the functional role of YAP1 associated EV, the present inventors treated naïve macrophages with EV isolated from YAP1KD and YAP1 WT cells. The present inventors' findings revealed that EVs from YAP1 KD cells induces M1 phenotype compared to M2 phenotype in the naïve macrophages (FIG. 30I). These findings indicate that the YAP1 regulated secreted EVs might play a role in the development of adaptive immunity.

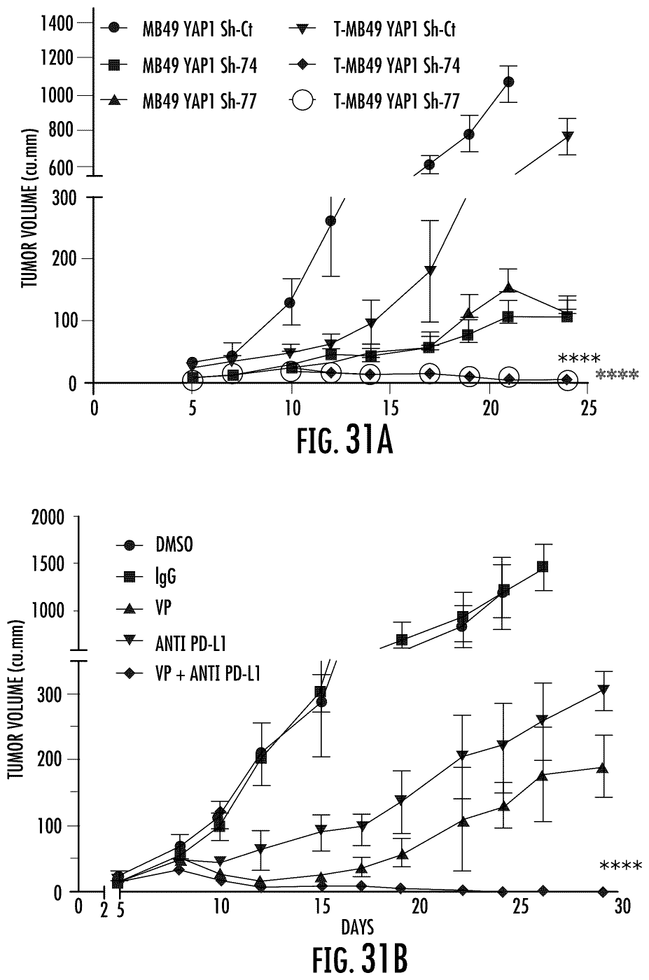

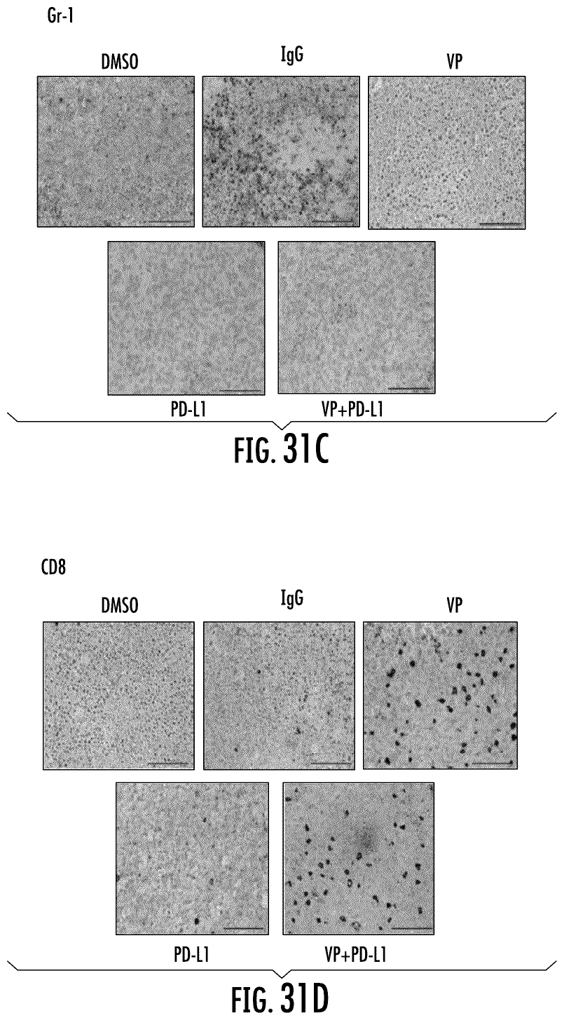

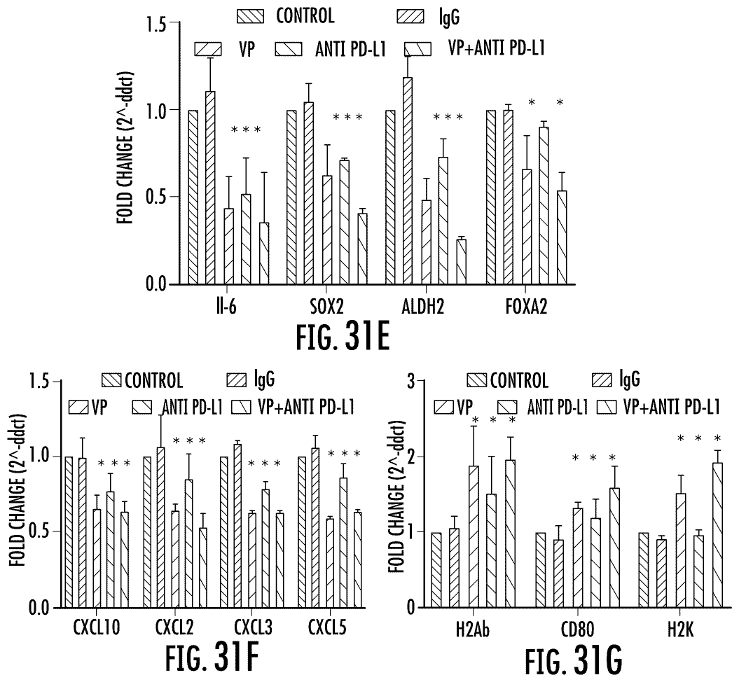

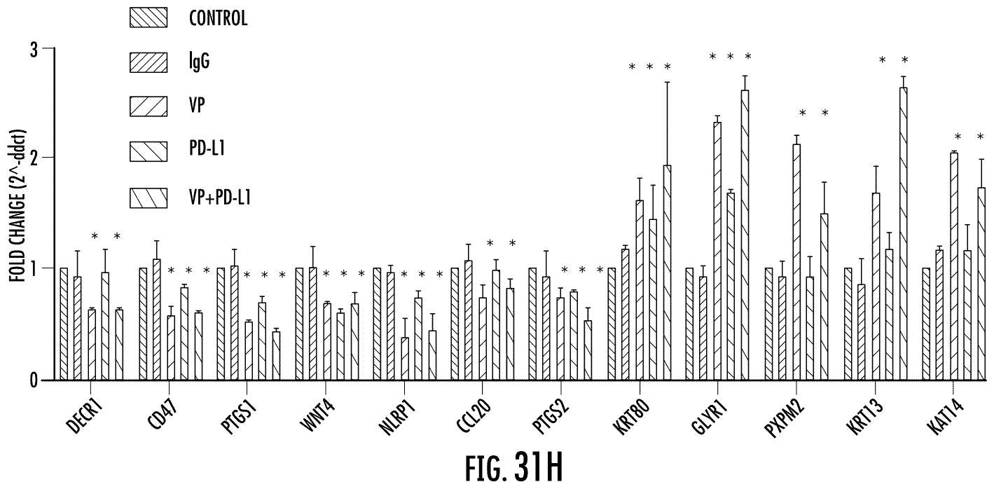

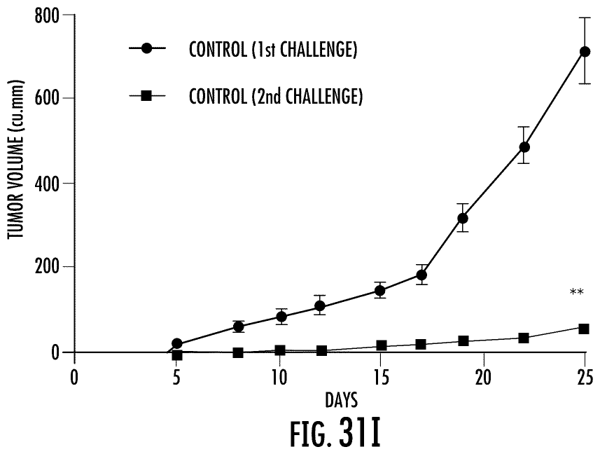

YAP1 inhibition combination with anti-PD-L1 showed synergistic antitumor efficacy. It was reported in different solid tumors that M2 macrophages and infiltration of MDSC in TME facilitate the cancer cells to gain resistance against immune checkpoint blockers (ICB) or immunotherapy (24, 41, 47). Since the present inventors' data indicate that YAP1 expression in the cancer cells induce the polarization of macrophages into M2 phenotype and infiltration of MDSC in the TME (FIG. 26), the present inventors hypothesize that ablation of the YAP1 signaling might results in increased efficacy of ICBs. Accordingly, the present inventors observed that anti-PD-L1 therapy was more effective in mice bearing the YAP1 KD tumors compared to the animals with the sh-control cells (FIG. 31A).

Pharmacologically, the present inventors explored the combinatorial therapeutic efficacy of anti PD-L1 antibody (ICB) and verteporfin (YAP1 inhibitor) in YAP1 expressing WT MB49 cell derived subcutaneous tumor in C57BL/6 mice. As expected, pharmacological inhibition of YAP1 in combination with anti-PDL1 antibody showed significant regression in the tumor growth (FIG. 31B) compared to any single therapy. At the end of 30 days treatment regime, the present inventors found no tumors in two animals out of five animals in the combinatorial drug treated group. The present inventors further maintain these animals until 62 days after the treatment and no tumor was found to appear in these mice. IHC analysis of the tumor tissue collected after euthanizing mice at the end of the treatment protocol (5 weeks) from each of the treated cohort showed decreased numbers of MDSC and increased CD8+ T cells in combination of YAP1 inhibition and anti-PD-L1 treated group compared to either of the single drug treated groups (FIG. 31C-31D). Quantitative RT-qPCR analysis showed decreased expression of several CSCs markers, IL-6 and CXCLs in combination drugs treated group compared to the each of the other group (FIG. 31E-31F). In addition, the combinatorial treatment regime also increases the level of cellular immunogenicity markers (H2-Ab, H2-K, and CD80) immune regulators (selected from the RNA sequencing data) in the tumor tissue (FIG. 31G-31H). To investigate the adaptive antitumor immunity memory in the animals that were previously treated with both verteporfin and anti PD-L1, the present inventors challenged subcutaneous tumor growth in control mice (no tumor was grown in these mice previously) and selected 2 previously drug treated mice that showed no tumor at the end of treatment protocol. Interestingly, the present inventors found that animals of previously drug treated group showed significant tumor growth inhibition compared to the control animals (FIG. 31I). Taken together, the present inventors' data led us to conclude that YAP1 may have a plausible role in immune therapy resistance and attenuation of YAP1 signaling might be a promising way to improve the efficacy of immunotherapy in UCB. Furthermore, YAP1 attenuation may have potential to develop adaptive antitumor immune memory.

### Discussion

The present inventors previously reported that decrease CSC promoting activity and increase therapeutic efficacy of chemotherapy in combination with YAP1 inhibition in bladder and lung cancer (7, 38). Here, the present inventors have investigated the role of YAP1 in modulating the urothelial tumor immune microenvironment (TIME). The present inventors' findings suggest that YAP1 induces immune suppression in UCB and comprehensive investigation of YAP1 regulated TIME led us to test the hypothesis that YAP1 inhibition combination with ICB might be a novel therapeutic strategy to treat selective UCB patients. The present inventors' mechanistic studies suggest that YAP1 expression facilitate immune evasion by the recruitment of MDSCs, polarization of macrophages and exhaustion of CD8+ T cells. MDSCs are regarded as one of the major drivers of immune evasion and development of resistance against ICB therapy (41, 48). The present inventors found that YAP1 expression induces the expression of IL6 and phosphorylation STAT3 in the UCB cells which is consistent with the present inventors' recent report in lung adenocarcinoma (LUAD) (38). YAP1 induces IL-6/STAT3 signaling that drives cancer stemness in LUAD (38) and different reports showed that generation of CSC are the primary step towards immune evasion (10, 11).

In the present study different in vivo studies using syngeneic mice and in vitro experiments with WT cells and YAP1 KD mouse bladder cancer cell lines showed that YAP1 is a critical determinant of immune evasion. Bioinformatic data from TCGA and molecular analysis of cell derived xenografts showed that high YAP1 expression is correlated with high MDSC signatures in the TME. YAP1 induces the infiltration of MDSCs and decreases the CD8 T cells in the TME. Further WT MB49 CDX analysis from anti-MDSC antibody treated animals showed increase of CD8 T cells in the TME along with tumor regression. Therefore, increase infiltration of CD8 T cells were expected in YAP1 KD MB49 CDX TME due to decrease infiltration of MDSC. Previously, researchers have identified immunoregulatory role of different oncogenes like KRAS, cMYC etc. (23, 49) and also identified potential role of YAP1 in regulating the infiltration of MDSCs and CD8+ T cells in other cancer types (50).

The present inventors have found STAT3, and IL-6 as potential downstream effectors of YAP1 induced tumorigenesis in UCB and IL-6/STAT3 expression was negatively associated with CD8 T cell infiltration and positively associated with MDSC infiltration. Recently different researchers also that IL-6 STAT3 signaling cascade is driving factor in the induction of “cold” TME through the regulation of MDSCs and exhaustion of CD8+ T cells (51). However, TCGA data analysis showed that there is very poor correlation between CD8 T cell infiltration and IL-6 expression in the TME. The IL-6/STAT3 were found to be activated by multiple signaling in many solid cancers as well as in different cell types and is associated with poor prognosis (11). IL-6 was also shown to regulate MDSCs and CD8T cell activity in TME (52). The discrepancy between the present inventors' preclinical data and TCGA data about the association of IL-6/STAT3 and CD8 T cell infiltration may be due to multiple cellular components in TCGA data compared to the present inventors' CDX model. A pure genetic model for UCB may allow us to appropriately study the signaling dynamics. Different carcinogen induced and engraftment models are highly accepted in studying UCB but compared to other cancer types UCB is underrepresented by Genetically engineered mouse (GEM) models (53).

The present inventors' data indicate that YAP1 induced STAT3/IL-6 signaling regulates several CXCR2 associated ligands such as CXCL2, CXCL3, CXCL5, CXCL8, CXCL10 and induces polarization of the naïve macrophages into M2 phenotype (FIG. 26). Accumulated evidence suggested that the coordinated action of these signaling intermediates and their interaction with different immune cell types facilitate immune evasion in UCB and other solid tumors such as lung cancer and breast cancer (54, 55). The present inventors found that YAP1 knockdown results in downregulation of several CXCR2 associated ligands in cancer cells and in xenograft tumors. It was reported that overexpression of some of CXCR2 associated ligands are linked with non-responsiveness to immune therapy in Pancreatic Ductal Adenocarcinoma (56). Therefore, although further study needed, it is likely that inhibition of YAP1 may enhance therapeutic efficacy of ICB by decreasing CXCR2 and its associated legends in UCB and other YAP1 induced cancer subtypes such as LUAD. To clarify the link of YAP1-IL6-STAT3-CXCR2 signaling for inducing immunosuppressive TME, the present inventors treated WTMB49 derived CDX bearing mice with STAT3 inhibitor. The present inventors' findings indicate that inhibition of STAT3 downregulate different CXCR2 associated ligands, and this observation led us to conclude that YAP1 regulates the expression of CXCLs through the STAT3 mediated signaling in UCB and open a new direction to explore whether YAP1 inhibition induces the adaptive immunity and improve the efficacy of immunotherapy or ICBs.

The known mechanisms of YAP1 regulated immune modulation still in its infancy. Strikingly, simultaneous injection of YAP1 KD cells and WT cancer cells (MB49) in the same mice, showed decreased growth of the WT cell derived xenograft compared to stand alone WT xenograft grown in separate mice. Tumor histological analysis and molecular analysis also suggest that there is decreased expression IL-6 and CXCLs and increased ratio of MDSC/CD8 T cells in the WT CDX simultaneously injected with the YAP1 KD cells compared to the WT cell-derived tumors in separate mice. Although the present inventors did not exclusively explore the target/targets for the noticeably slower growth of WT tumors in simultaneously YAP1 KD and WT tumors in the same mice, the present inventors' experiments revealed that YAP1 KD cells release significantly more extracellular vesicles (EVs) in the cell culture media compared to the WT cells and these EVs are loaded with more proteins. Further analysis of these EV in future will help us to determine YAP1 regulated targets for the slower growth of WT tumors where YAP1 KD tumors were grown in the opposite flank of the same mice. This observation can be a plausible explanation to the antitumor response of YAP1 inhibition reported recently (30). Differences in component proteins and RNA in the EVs of YAP1 KD and WT cells will open up a fertile avenue to investigate the mechanism of induction of adaptive immunity by the YAP1 KD UCB cells. Apart the these, the present inventors' data also showed that YAP1 expression is correlated with the intracellular accumulation of lipid droplets (LDs) (FIG. 29). In the TME LDs are shown to have potential to induce cancer stemness and immunosuppression by regulating the macrophage phenotype (57, 58).

Recent clinical studies agreed upon the fact that ICB therapy is one of the effective ways to treat cancers including UCB (58). However, there are many reports indicating that a major percentage of patients do not respond to the ICB therapy specifically some GU cancers such as UCB and prostate cancer (59, 60). Different mechanisms of immune evasion have been identified in different cancer types for the non-responsiveness to ICB such as T-cell exhaustion, macrophage polarization, deregulation of IFNs and ILs signaling, recruitment of immunosuppressive cells in the TME (60). However, no significant progress has been achieved to enhance ICB response. Compared to other cancer types, only few studies have been conducted in UCB to decipher the underlying mechanism of non-responsiveness of ICB (61, 62). The present inventors' results indicate a definite role of YAP1 in regulating immune signaling and induction of immunologically cold TIME in UCB. Although further study needed, the present inventors observed that YAP1 induces immunosuppressive TIME by modulating the expression of key signaling molecules such as DECR1, CD47, PTGS1, PTGS2, WNT4, NLRP1, CCL20, KRT80, GLYR1, PXPM2, KRT13, KAT14, MHC-I, MHC-II and CD80 (63-67). These expression pattern correlates with high MDSC recruitment and T cell exhaustion (68, 69). In addition, overexpression YAP1 induces the polarization of macrophages into M2 types/TAMs by regulating different genes such as iNOS, MerTK, IL-10, STAT3, CD163, CD206, Arg-1, CD86, TNF-α in the naïve macrophages. Altogether, the present inventors' findings suggest that immune evasion by YAP1 is a complex process and accumulated effect regulated by the YAP1 results in resistance to ICBs. Effective therapeutic strategies combining MDSC inhibitor and ICB have been tested in many clinical trials (such as NCT003302247) and preclinical studies have also been carried out combining CXCR2 inhibitor and ICB (23, 70). Currently phase 1 clinical trial has also been started in melanoma by combining SX-682 (CXCR1/2 inhibitor) with ICB (NCT03161431) (23, 39, 71). The present inventors have to wait to see the outcome of these clinical trial. However, the present inventors speculate YAP1 inhibition with ICB may be more effective as YAP1 has effect on MDSC infiltration, CXCR2 expression and macrophage polarization.

Different reports suggest that non-responsiveness to ICBs can often be attributed to the lack of immunogenicity in the TME and this greatly varies between individuals and cancer types (72). The present inventors' study revealed that downregulation of YAP1 in mouse bladder cancer cell lines, can induce “immunologically hot” TME in response to genetic and pharmacologic inhibition of YAP1. It allows us to hypothesize that YAP1 inhibition might be a possible way to enhance the efficacy of ICBs in UCB and other cancer types. Among different ICB, anti PD-L1 and anti-PD-1 has been reported to be the most effective agents in treating UCB (36, 73, 74). But due to lack of knowledge about the regulation of these targets in the tumor cells as well as in the immune cells, it is still difficult for the clinicians to choose the right option for the patients. Here the present inventors choose anti-PL1 and in YAP1 WTMB49 tumor bearing mice combinatorial administration of verteporfin and anti-PD-L1 showed synergistic effect in regressing tumor volume. Interestingly, slower growth of tumors was observed after challenging of verteporfin and anti-PD-L1 treated mice with fresh WTMB49 cells compared to tumor grown in non-treated mice. Although need to establish by future studies, this finding indicates that immune memory may develop in treated mice group.

The present inventors' findings in in vitro suggest that YAP1 inhibition induces the pathways associated with immune stimulation. RNA array-based findings were further supported by molecular analysis of the tumor tissues derived from mice treated with different treatment regimens. As for examples, combinatorial treatment with Verteporfin and anti-PDL1 increases the CD8/MDSS ratio in the tumor tissue compared to anti-PDL1 alone. Thus, combination of YAP1 inhibitor and anti-PD-L1 would be a plausible therapeutic approach for the patients whose tumor expresses YAP1. At present the present inventors do not have enough data to conclude whether YAP1 can be a marker for deciding this combinatorial therapy. Future clinical studies will explore this possibility. YAP1 inhibition with ICB should also be carried out on other YAP1 expressing cancers with poor immunogenic response. The shortcoming of the present inventors' study is very much prominent that the immune system and the immune response of mice greatly varies from the human system, and it is still remained to be elucidated if this therapeutic approach will be effective in a human system. Though in the present inventors' previous publications the present inventors found YAP1 inhibition with chemotherapy is effective against patient derived xenograft (PDX) of UCB and LUAD developed in NSG mice (38), in future the present inventors may grow PDX in humanized mice to get comparatively more human relevant data. Nonetheless, future studies exploring the therapeutic potential of modulating YAP1 and downstream molecules will have significant clinical importance.

### REFERENCES

1. Hayashi M, Guida E, Inokawa Y, Goldberg R, Reis L O, Ooki A, et al. GULP1 regulates the NRF2-KEAP1 signaling axis in urothelial carcinoma. Sci Signal. 2020; 13(645).
2. Shimwell N J, Bryan R T, Wei W, James N D, Cheng K K, Zeegers M P, et al. Combined proteome and transcriptome analyses for the discovery of urinary biomarkers for urothelial carcinoma. Br J Cancer. 2013; 108(9):1854-61.
3. Sun M, Abdollah F, Bianchi M, Trinh Q D, Shariat S F, Jeldres C, et al. Conditional survival of patients with urothelial carcinoma of the urinary bladder treated with radical cystectomy. Eur J Cancer. 2012; 48(10):1503-11.
4. Malladi S, Macalinao D G, Jin X, He L, Basnet H, Zou Y, et al. Metastatic Latency and Immune Evasion through Autocrine Inhibition of WNT. Cell. 2016; 165(1):45-60.
5. Miao Y, Yang H, Levorse J, Yuan S, Polak L, Sribour M, et al. Adaptive Immune Resistance Emerges from Tumor-Initiating Stem Cells. Cell. 2019; 177(5):1172-86.e14.
6. Su R, Dong L, Li Y, Gao M, Han L, Wunderlich M, et al. Targeting FTO Suppresses Cancer Stem Cell Maintenance and Immune Evasion. Cancer Cell. 2020; 38(1):79-96.e11.
7. Ooki A, Pena M D C R, Marchionni L, Dinalankara W, Begum A, Hahn N M, et al. YAP1 and COX2 coordinately regulate urothelial cancer stem-like cells. Cancer research. 2018; 78(1):168-81.
8. Shibata M, Ham K, Hoque M O. A time for YAP1: Tumorigenesis, immunosuppression and targeted therapy. International journal of cancer. 2018; 143(9):2133-44.
9. Shibata M, Hoque M O. Targeting cancer stem cells: a strategy for effective eradication of cancer. Cancers. 2019; 11(5):732.
10. Chan L C, Li C W, Xia W, Hsu J M, Lee H H, Cha J H, et al. IL-6/JAK1 pathway drives PD-L1 Y112 phosphorylation to promote cancer immune evasion. J Clin Invest. 2019; 129(8):3324-38.
11. Johnson D E, O'Keefe R A, Grandis J R. Targeting the IL-6/JAK/STAT3 signalling axis in cancer. Nat Rev Clin Oncol. 2018; 15(4):234-48.
12. Lee H, Chin H, Kim H, Jung H, Lee D. STAT3-mediated MLST8 gene expression regulates cap-dependent translation in cancer cells. Mol Oncol. 2020; 14(8):1850-67.
13. Kogan D, Grabner A, Yanucil C, Faul C, Ulaganathan V K. STAT3-enhancing germline mutations contribute to tumor-extrinsic immune evasion. J Clin Invest. 2018; 128(5):1867-72.
14. Sánchez-Zauco N, Torres J, Gómez A, Camorlinga-Ponce M, Muñoz-Perez L, Herrera-Goepfert R, et al. Circulating blood levels of IL-6, IFN-γ, and IL-10 as potential diagnostic biomarkers in gastric cancer: a controlled study. BMC cancer. 2017; 17(1):1-10.
15. Unver N, McAllister F. IL-6 family cytokines: Key inflammatory mediators as biomarkers and potential therapeutic targets. Cytokine & growth factor reviews. 2018; 41:10-7.
16. Sahibzada H A, Khurshid Z, Khan R S, Naseem M, Siddique K M, Mali M, et al. Salivary IL-8, IL-6 and TNF-α as potential diagnostic biomarkers for oral cancer. Diagnostics. 2017; 7(2):21.
17. Liu Y, Fuchs J, Li C, Lin J. IL-6, a risk factor for hepatocellular carcinoma: FLLL32 inhibits IL-6-induced STAT3 phosphorylation in human hepatocellular cancer cells. Cell cycle. 2010; 9(17):3423-7.
18. Chen Y-Q, Li P-C, Pan N, Gao R, Wen Z-F, Zhang T-Y, et al. Tumor-released autophagosomes induces CD4+ T cell-mediated immunosuppression via a TLR2-IL-6 cascade. Journal for immunotherapy of cancer. 2019; 7(1):1-16.
19. Fu X-L, Duan W, Su C-Y, Mao F-Y, Lv Y-P, Teng Y-S, et al. Interleukin 6 induces M2 macrophage differentiation by STAT3 activation that correlates with gastric cancer progression. Cancer Immunology, Immunotherapy. 2017; 66(12):1597-608.
20. Yin Z, Ma T, Lin Y, Lu X, Zhang C, Chen S, et al. IL-6/STAT3 pathway intermediates M1/M2 macrophage polarization during the development of hepatocellular carcinoma. Journal of cellular biochemistry. 2018; 119(11):9419-32.
21. Weber R, Groth C, Lasser S, Arkhypov I, Petrova V, Altevogt P, et al. IL-6 as a major regulator of MDSC activity and possible target for cancer immunotherapy. Cellular Immunology. 2021; 359:104254.
22. Weber R, Riester Z, Huser L, Sticht C, Siebenmorgen A, Groth C, et al. IL-6 regulates CCR5 expression and immunosuppressive capacity of MDSC in murine melanoma. Journal for immunotherapy of cancer. 2020; 8(2).
23. Liao W, Overman M J, Boutin A T, Shang X, Zhao D, Dey P, et al. KRAS-IRF2 Axis Drives Immune Suppression and Immune Therapy Resistance in Colorectal Cancer. Cancer Cell. 2019; 35(4):559-72.e7.
24. DeNardo D G, Ruffell B. Macrophages as regulators of tumour immunity and immunotherapy. Nat Rev Immunol. 2019; 19(6):369-82.
25. Pan Y, Yu Y, Wang X, Zhang T. Tumor-Associated Macrophages in Tumor Immunity. Front Immunol. 2020; 11:583084.
26. De Cicco P, Ercolano G, Ianaro A. The New Era of Cancer Immunotherapy: Targeting Myeloid-Derived Suppressor Cells to Overcome Immune Evasion. Frontiers in Immunology. 2020; 11(1680).
27. Cheng Y, Ma X L, Wei Y Q, Wei X W. Potential roles and targeted therapy of the CXCLs/CXCR2 axis in cancer and inflammatory diseases. Biochim Biophys Acta Rev Cancer. 2019; 1871(2):289-312.
28. Ostrand-Rosenberg S, Sinha P. Myeloid-derived suppressor cells: linking inflammation and cancer. J Immunol. 2009; 182(8):4499-506.
29. Duan Z, Luo Y. Targeting macrophages in cancer immunotherapy. Signal Transduction and Targeted Therapy. 2021; 6(1):127.
30. Moroishi T, Hayashi T, Pan W W, Fujita Y, Holt M V, Qin J, et al. The Hippo Pathway Kinases LATS1/2 Suppress Cancer Immunity. Cell. 2016; 167(6):1525-39.e17.
31. Tirinato L, Pagliari F, Limongi T, Marini M, Falqui A, Seco J, et al. An overview of lipid droplets in cancer and cancer stem cells. Stem cells international. 2017; 2017.
32. Cruz A L, Barreto EdA, Fazolini N P, Viola J P, Bozza P T. Lipid droplets: platforms with multiple functions in cancer hallmarks. Cell death & disease. 2020; 11(2):1-16.
33. Rouanne M, Bajorin D F, Hannan R, Galsky M D, Williams S B, Necchi A, et al. Rationale and Outcomes for Neoadjuvant Immunotherapy in Urothelial Carcinoma of the Bladder. Eur Urol Oncol. 2020; 3(6):728-38.
34. Siefker-Radtke A O, Apolo A B, Bivalacqua T J, Spiess P E, Black P C. Immunotherapy with Checkpoint Blockade in the Treatment of Urothelial Carcinoma. J Urol. 2018; 199(5):1129-42.
35. Siefker-Radtke A, Curti B. Immunotherapy in metastatic urothelial carcinoma: focus on immune checkpoint inhibition. Nat Rev Urol. 2018; 15(2):112-24.
36. Lopez-Beltran A, Cimadamore A, Blanca A, Massari F, Vau N, Scarpelli M, et al. Immune Checkpoint Inhibitors for the Treatment of Bladder Cancer. Cancers (Basel). 2021; 13(1).
37. Shibata M, Ham K, Hoque M O. A time for YAP1: Tumorigenesis, immunosuppression and targeted therapy. Int J Cancer. 2018; 143(9):2133-44.
38. Shibata M, Ooki A, Inokawa Y, Sadhukhan P, Ugurlu M T, Izumchenko E, et al. Concurrent Targeting of Potential Cancer Stem Cells Regulating Pathways Sensitizes Lung Adenocarcinoma to Standard Chemotherapy. Molecular Cancer Therapeutics. 2020; 19(10):2175-85.
39. Highfill S L, Cui Y, Giles A J, Smith J P, Zhang H, Morse E, et al. Disruption of CXCR2-mediated MDSC tumor trafficking enhances anti-PD1 efficacy. Sci Transl Med. 2014; 6(237):237ra67.
40. Zhang Y, Guan X-y, Jiang P. Cytokine and Chemokine Signals of T-Cell Exclusion in Tumors. Frontiers in Immunology. 2020; 11(3093).
41. Jiang M, Chen J, Zhang W, Zhang R, Ye Y, Liu P, et al. Interleukin-6 Trans-Signaling Pathway Promotes Immunosuppressive Myeloid-Derived Suppressor Cells via Suppression of Suppressor of Cytokine Signaling 3 in Breast Cancer. Front Immunol. 2017; 8:1840.
42. Zhang Z, Liu S, Zhang B, Qiao L, Zhang Y, Zhang Y. T Cell Dysfunction and Exhaustion in Cancer. Frontiers in Cell and Developmental Biology. 2020; 8(17).
43. Teijeira A, Garasa S, Gato M, Alfaro C, Migueliz I, Cirella A, et al. CXCR1 and CXCR2 Chemokine Receptor Agonists Produced by Tumors Induce Neutrophil Extracellular Traps that Interfere with Immune Cytotoxicity. Immunity. 2020; 52(5):856-71.e8.
44. Shang C, Qiao J, Guo H. The dynamic behavior of lipid droplets in the pre-metastatic niche. Cell Death Dis. 2020; 11(11):990.
45. Scifres C M, Chen B, Nelson D M, Sadovsky Y. Fatty acid binding protein 4 regulates intracellular lipid accumulation in human trophoblasts. J Clin Endocrinol Metab. 2011; 96(7):E1083-91.
46. Rozeveld C N, Johnson K M, Zhang L, Razidlo G L. KRAS Controls Pancreatic Cancer Cell Lipid Metabolism and Invasive Potential through the Lipase HSL. Cancer Res. 2020; 80(22):4932-45.
47. Xiang X, Wang J, Lu D, Xu X. Targeting tumor-associated macrophages to synergize tumor immunotherapy. Signal Transduction and Targeted Therapy. 2021; 6(1):75.
48. Li K, Shi H, Zhang B, Ou X, Ma Q, Chen Y, et al. Myeloid-derived suppressor cells as immunosuppressive regulators and therapeutic targets in cancer. Signal Transduction and Targeted Therapy. 2021; 6(1):362.
49. Ren X, Xiao J, Zhang W, Wang F, Yan Y, Wu X, et al. Inhibition of CCL7 derived from Mo-MDSCs prevents metastatic progression from latency in colorectal cancer. Cell Death & Disease. 2021; 12(5):484.
50. Wang G, Lu X, Dey P, Deng P, Wu C C, Jiang S, et al. Targeting YAP-Dependent MDSC Infiltration Impairs Tumor Progression. Cancer Discov. 2016; 6(1):80-95.
51. Smith A D, Lu C, Payne D, Paschall A V, Klement J D, Redd P S, et al. Autocrine IL6-Mediated Activation of the STAT3-DNMT Axis Silences the TNFα-RIP1 Necroptosis Pathway to Sustain Survival and Accumulation of Myeloid-Derived Suppressor Cells. Cancer Res. 2020; 80(15):3145-56.
52. Kato T, Noma K, Ohara T, Kashima H, Katsura Y, Sato H, et al. Cancer-Associated Fibroblasts Affect Intratumoral CD8(+) and FoxP3(+) T Cells Via IL6 in the Tumor Microenvironment. Clin Cancer Res. 2018; 24(19):4820-33.
53. Kobayashi T, Owczarek T B, McKieman J M, Abate-Shen C. Modelling bladder cancer in mice: opportunities and challenges. Nat Rev Cancer. 2015; 15(1):42-54.
54. Altorki N K, Markowitz G J, Gao D, Port J L, Saxena A, Stiles B, et al. The lung microenvironment: an important regulator of tumour growth and metastasis. Nat Rev Cancer. 2019; 19(1):9-31.
55. Qi M, Xia Y, Wu Y, Zhang Z, Wang X, Lu L, et al. Lin28B-high breast cancer cells promote immune suppression in the lung pre-metastatic niche via exosomes and support cancer progression. Nat Commun. 2022; 13(1):897.
56. Chao T, Furth E E, Vonderheide R H. CXCR2-Dependent Accumulation of Tumor-Associated Neutrophils Regulates T-cell Immunity in Pancreatic Ductal Adenocarcinoma. Cancer Immunol Res. 2016; 4(11):968-82.
57. Visweswaran M, Arfuso F, Warner S, Dharmarajan A. Aberrant lipid metabolism as an emerging therapeutic strategy to target cancer stem cells. Stem Cells. 2020; 38(1):6-14.
58. Wu H, Han Y, Rodriguez Sillke Y, Deng H, Siddiqui S, Treese C, et al. Lipid droplet-dependent fatty acid metabolism controls the immune suppressive phenotype of tumor-associated macrophages. EMBO Mol Med. 2019; 11(11):e10698.
59. Sweis R F, Galsky M D. Emerging role of immunotherapy in urothelial carcinoma-Immunobiology/biomarkers. Urol Oncol. 2016; 34(12):556-65.
60. Lee Y C, Lam H M, Rosser C, Theodorescu D, Parks W C, Chan K S. The dynamic roles of the bladder tumour microenvironment. Nat Rev Urol. 2022.
61. van den Bulk J, Verdegaal E M, de Miranda N F. Cancer immunotherapy: broadening the scope of targetable tumours. Open Biol. 2018; 8(6).
62. Duffy M J, Crown J. Biomarkers for Predicting Response to Immunotherapy with Immune Checkpoint Inhibitors in Cancer Patients. Clin Chem. 2019; 65(10):1228-38.
63. Chao M P, Weissman I L, Majeti R. The CD47-SIRPα pathway in cancer immune evasion and potential therapeutic implications. Curr Opin Immunol. 2012; 24(2):225-32.
64. Liu B, Qu L, Yan S. Cyclooxygenase-2 promotes tumor growth and suppresses tumor immunity. Cancer Cell International. 2015; 15(1):106.
65. IL18 Promotes MDSC-Mediated Immunosuppression in Multiple Myeloma. Cancer Discovery. 2018; 8(5):OF12-OF.
66. Kadomoto S, Izumi K, Mizokami A. The CCL20-CCR6 Axis in Cancer Progression. Int J Mol Sci. 2020; 21(15).
67. Hu Z, Long T, Ma Y, Zhu J, Gao L, Zhong Y, et al. Downregulation of GLYR1 contributes to microsatellite instability colorectal cancer by targeting p21 via the p38MAPK and PI3K/AKT pathways. J Exp Clin Cancer Res. 2020; 39(1):76.
68. Korbecki J, Grochans S, Gutowska I, Barczak K, Baranowska-Bosiacka I. CC Chemokines in a Tumor: A Review of Pro-Cancer and Anti-Cancer Properties of Receptors CCR5, CCR6, CCR7, CCR8, CCR9, and CCR10 Ligands. Int J Mol Sci. 2020; 21(20).
69. Belk J A, Daniel B, Satpathy A T. Epigenetic regulation of T cell exhaustion. Nat Immunol. 2022; 23(6):848-60.
70. Lu X, Lu X. Enhancing immune checkpoint blockade therapy of genitourinary malignancies by co-targeting PMN-MDSCs. Biochim Biophys Acta Rev Cancer. 2022; 1877(3):188702.
71. Yang J, Yan C, Vilgelm A E, Chen S C, Ayers G D, Johnson C A, et al. Targeted Deletion of CXCR2 in Myeloid Cells Alters the Tumor Immune Environment to Improve Antitumor Immunity. Cancer Immunol Res. 2021; 9(2):200-13.
72. Minnar C M, Chariou P L, Horn L A, Hicks K C, Palena C, Schlom J, et al. Tumor-targeted interleukin-12 synergizes with entinostat to overcome PD-1/PD-L1 blockade-resistant tumors harboring MHC-I and APM deficiencies. J Immunother Cancer. 2022; 10(6).
73. Rizzo A, Mollica V, Massari F. Expression of Programmed Cell Death Ligand 1 as a Predictive Biomarker in Metastatic Urothelial Carcinoma Patients Treated with First-line Immune Checkpoint Inhibitors Versus Chemotherapy: A Systematic Review and Meta-analysis. Eur Urol Focus. 2021.
74. Kirschner A N, Wang J, Rajkumar-Calkins A, Neuzil K E, Chang S S. Intravesical Anti-PD-1 Immune Checkpoint Inhibition Treats Urothelial Bladder Cancer in a Mouse Model. J Urol. 2021; 205(5):1336-43.

1. A method for treating cancer in a subject in need thereof comprising the step of administering to the subject an effective amount of a YAP1 inhibitor and a STAT3 inhibitor.

2. The method of claim 1, wherein the subject is further treated with chemotherapy.

3. The method of claim 1, wherein the subject is further treated with immunotherapy.

4. The method of claim 2, wherein the YAP1 inhibitor and STAT3 inhibitor increase sensitivity of the cancer in the subject to chemotherapy.

5. The method of claim 3, wherein the YAP1 inhibitor and STAT3 inhibitor increase sensitivity of the cancer in the subject to immunotherapy.

6. A method for increasing sensitivity of cancer in a subject to chemotherapy and/or immunotherapy comprising the step of administering to the subject an effective amount of a YAP1 inhibitor and a STAT3 inhibitor.

7. The method of claim 6, further comprising the step of administering to the subject chemotherapy and/or immunotherapy.

8. The method of claim 6, wherein the YAP1 inhibitor comprises verteporfin or a derivative thereof.

9. The method of claim 6, wherein the STAT3 inhibitor comprises S3I-201 or a derivative thereof.

10. The method of claim 6, wherein the cancer comprises lung adenocarcinoma or urothelial carcinoma of the bladder.

11-23. (canceled)
